SEQUENCE LISTING (38 sequences)

SEQ ID NO: 1 - protein, 349 aa, Mus musculus
GGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSR

SEQ ID NO: 2 - protein, 377 aa, Mus musculus
FSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVPISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATP

SEQ ID NO: 3 - protein, 495 aa, Mus musculus
ARGRSGNRTLNAGAVGGRRAGGALARGGRELNSTARASGVPEAGSRRGQSAAAAAAAAAAASATVTYETCWGYYDVSGQYDKEFECNNSESGYLYCCGTCYYRFCCKKRHEKLDQRQCTNYQSPVWVQTPSTKVVSPGPENKYDPEKDKTNFTVYITCGVIAFVIVAGVFAKVSYDKAHRPPREMNIHRALADILRQQGPIPIAHCERETISAIDTSPKENTPVRSTSKNHYTPVRTAKQTPGDRQYNHPILSSATQTPTHEKPRMNNILTSATEPYDLSFSRSYQNLAHLPPSYESAVKTNPSKYSSLKRLTDKEADEYYMRRRHLPDLAARGTLPLNVIQMSQQKPLPRERPRRPIRAMSQDRVLSPRRGLPDEFGMPYDRILSDEQLLSTERLHSQDPLLSPERTAFPEQSLSRAISHTDVFVSTPVLDRYRMTKMHSHPSASNNSYATLGQSQTAAKRHAFASRRHNTVEQLHYIPGHHTCYTASKTEVTV

SEQ ID NO: 4 - protein, 536 aa, Mus musculus
RPSNDTSSVAPGPLPALLAHLRRLTGALAGGGSAAGTSANATKTSPASGTGAAARAPPPAELCHGYYDVMGQYDATFNCSTGSYRFCCGTCHYRFCCEHRHMRLAQASCSNYDTPRWATTPPPLAGGAGGAGGAGGGPGPGQAGWLEGGRAGGAGGRGGEGPGGSTAYVVCGVISFALAVGVGAKVAFSKASRAPRAHREINVPRALVDILRHQAGPATRPDRARSSSLTPGLGGPDSMAPRTPKNLYNTMKPSNLDNLHYNVNSPKHHAATLDWRAMPPPSPSLHYSTLSCSRSFHNLSHLPPSYEAAVKSELNRYSSLKRLAEKDLDEAYLKRRQLEMPRGTLPLHALRRPGTGGGYRMDGWGGPEELGLAPAPNPRRVMSQEHLLGDGSRASRYEFTLPRARLVSQEHLLLSSPEALRQSREHLLSPPRSPALPPDPTTRASLAASHSNLLLGPGGPPTPLHGLPPSGLHAHHHHALHGSPQPAWMSDAGGGGGTLARRPPFQRQGTLEQLQFIPGHHLPQHLRTASKNEVTV

SEQ ID NO: 5 - protein, 6 aa, unidentified
  source 1..6: note = Synthetic construct
GSGSGS

SEQ ID NO: 6 - protein, 30 aa, Mus musculus
MALRRLLLPPLLLSLLLSLASLHLPPGADA

SEQ ID NO: 7 - protein, 22 aa, Mus musculus
MPALLLLGTVALLASAAGPAGA

SEQ ID NO: 8 - protein, 9 aa, synthetic construct
YPYDVPDYA

SEQ ID NO: 9 - protein, 850 aa, synthetic construct
GGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRGSGSGSARGRSGNRTLNAGAVGGRRAGGALARGGRELNSTARASGVPEAGSRRGQSAAAAAAAAAAASATVTYETCWGYYDVSGQYDKEFECNNSESGYLYCCGTCYYRFCCKKRHEKLDQRQCTNYQSPVWVQTPSTKVVSPGPENKYDPEKDKTNFTVYITCGVIAFVIVAGVFAKVSYDKAHRPPREMNIHRALADILRQQGPIPIAHCERETISAIDTSPKENTPVRSTSKNHYTPVRTAKQTPGDRQYNHPILSSATQTPTHEKPRMNNILTSATEPYDLSFSRSYQNLAHLPPSYESAVKTNPSKYSSLKRLTDKEADEYYMRRRHLPDLAARGTLPLNVIQMSQQKPLPRERPRRPIRAMSQDRVLSPRRGLPDEFGMPYDRILSDEQLLSTERLHSQDPLLSPERTAFPEQSLSRAISHTDVFVSTPVLDRYRMTKMHSHPSASNNSYATLGQSQTAAKRHAFASRRHNTVEQLHYIPGHHTCYTASKTEVTV

SEQ ID NO: 10 - protein, 880 aa, synthetic construct
MALRRLLLPPLLLSLLLSLASLHLPPGADAGGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRGSGSGSARGRSGNRTLNAGAVGGRRAGGALARGGRELNSTARASGVPEAGSRRGQSAAAAAAAAAAASATVTYETCWGYYDVSGQYDKEFECNNSESGYLYCCGTCYYRFCCKKRHEKLDQRQCTNYQSPVWVQTPSTKVVSPGPENKYDPEKDKTNFTVYITCGVIAFVIVAGVFAKVSYDKAHRPPREMNIHRALADILRQQGPIPIAHCERETISAIDTSPKENTPVRSTSKNHYTPVRTAKQTPGDRQYNHPILSSATQTPTHEKPRMNNILTSATEPYDLSFSRSYQNLAHLPPSYESAVKTNPSKYSSLKRLTDKEADEYYMRRRHLPDLAARGTLPLNVIQMSQQKPLPRERPRRPIRAMSQDRVLSPRRGLPDEFGMPYDRILSDEQLLSTERLHSQDPLLSPERTAFPEQSLSRAISHTDVFVSTPVLDRYRMTKMHSHPSASNNSYATLGQSQTAAKRHAFASRRHNTVEQLHYIPGHHTCYTASKTEVTV

SEQ ID NO: 11 - protein, 889 aa, synthetic construct
MALRRLLLPPLLLSLLLSLASLHLPPGADAGGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRGSGSGSARGRSGNRTLNAGAVGGRRAGGALARGGRELNSTARASGVPEAGSRRGQSAAAAAAAAAAASATVTYETCWGYYDVSGQYDKEFECNNSESGYLYCCGTCYYRFCCKKRHEKLDQRQCTNYQSPVWVQTPSTKVVSPGPENKYDPEKDKTNFTVYITCGVIAFVIVAGVFAKVSYDKAHRPPREMNIHRALADILRQQGPIPIAHCERETISAIDTSPKENTPVRSTSKNHYTPVRTAKQTPGDRQYNHPILSSATQTPTHEKPRMNNILTSATEPYDLSFSRSYQNLAHLPPSYESAVKTNPSKYSSLKRLTDKEADEYYMRRRHLPDLAARGTLPLNVIQMSQQKPLPRERPRRPIRAMSQDRVLSPRRGLPDEFGMPYDRILSDEQLLSTERLHSQDPLLSPERTAFPEQSLSRAISHTDVFVSTPVLDRYRMTKMHSHPSASNNSYATLGQSQTAAKRHAFASRRHNTVEQLHYIPGHHTCYTASKTEVTVYPYDVPDYA

SEQ ID NO: 12 - protein, 891 aa, synthetic construct
GGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRGSGSGSRPSNDTSSVAPGPLPALLAHLRRLTGALAGGGSAAGTSANATKTSPASGTGAAARAPPPAELCHGYYDVMGQYDATFNCSTGSYRFCCGTCHYRFCCEHRHMRLAQASCSNYDTPRWATTPPPLAGGAGGAGGAGGGPGPGQAGWLEGGRAGGAGGRGGEGPGGSTAYVVCGVISFALAVGVGAKVAFSKASRAPRAHREINVPRALVDILRHQAGPATRPDRARSSSLTPGLGGPDSMAPRTPKNLYNTMKPSNLDNLHYNVNSPKHHAATLDWRAMPPPSPSLHYSTLSCSRSFHNLSHLPPSYEAAVKSELNRYSSLKRLAEKDLDEAYLKRRQLEMPRGTLPLHALRRPGTGGGYRMDGWGGPEELGLAPAPNPRRVMSQEHLLGDGSRASRYEFTLPRARLVSQEHLLLSSPEALRQSREHLLSPPRSPALPPDPTTRASLAASHSNLLLGPGGPPTPLHGLPPSGLHAHHHHALHGSPQPAWMSDAGGGGGTLARRPPFQRQGTLEQLQFIPGHHLPQHLRTASKNEVTV

SEQ ID NO: 13 - protein, 913 aa, synthetic construct
MPALLLLGTVALLASAAGPAGAGGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRGSGSGSRPSNDTSSVAPGPLPALLAHLRRLTGALAGGGSAAGTSANATKTSPASGTGAAARAPPPAELCHGYYDVMGQYDATFNCSTGSYRFCCGTCHYRFCCEHRHMRLAQASCSNYDTPRWATTPPPLAGGAGGAGGAGGGPGPGQAGWLEGGRAGGAGGRGGEGPGGSTAYVVCGVISFALAVGVGAKVAFSKASRAPRAHREINVPRALVDILRHQAGPATRPDRARSSSLTPGLGGPDSMAPRTPKNLYNTMKPSNLDNLHYNVNSPKHHAATLDWRAMPPPSPSLHYSTLSCSRSFHNLSHLPPSYEAAVKSELNRYSSLKRLAEKDLDEAYLKRRQLEMPRGTLPLHALRRPGTGGGYRMDGWGGPEELGLAPAPNPRRVMSQEHLLGDGSRASRYEFTLPRARLVSQEHLLLSSPEALRQSREHLLSPPRSPALPPDPTTRASLAASHSNLLLGPGGPPTPLHGLPPSGLHAHHHHALHGSPQPAWMSDAGGGGGTLARRPPFQRQGTLEQLQFIPGHHLPQHLRTASKNEVTV

SEQ ID NO: 14 - protein, 922 aa, synthetic construct
MPALLLLGTVALLASAAGPAGAGGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRGSGSGSRPSNDTSSVAPGPLPALLAHLRRLTGALAGGGSAAGTSANATKTSPASGTGAAARAPPPAELCHGYYDVMGQYDATFNCSTGSYRFCCGTCHYRFCCEHRHMRLAQASCSNYDTPRWATTPPPLAGGAGGAGGAGGGPGPGQAGWLEGGRAGGAGGRGGEGPGGSTAYVVCGVISFALAVGVGAKVAFSKASRAPRAHREINVPRALVDILRHQAGPATRPDRARSSSLTPGLGGPDSMAPRTPKNLYNTMKPSNLDNLHYNVNSPKHHAATLDWRAMPPPSPSLHYSTLSCSRSFHNLSHLPPSYEAAVKSELNRYSSLKRLAEKDLDEAYLKRRQLEMPRGTLPLHALRRPGTGGGYRMDGWGGPEELGLAPAPNPRRVMSQEHLLGDGSRASRYEFTLPRARLVSQEHLLLSSPEALRQSREHLLSPPRSPALPPDPTTRASLAASHSNLLLGPGGPPTPLHGLPPSGLHAHHHHALHGSPQPAWMSDAGGGGGTLARRPPFQRQGTLEQLQFIPGHHLPQHLRTASKNEVTVYPYDVPDYA

SEQ ID NO: 15 - protein, 878 aa, synthetic construct
FSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVPISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATPGSGSGSARGRSGNRTLNAGAVGGRRAGGALARGGRELNSTARASGVPEAGSRRGQSAAAAAAAAAAASATVTYETCWGYYDVSGQYDKEFECNNSESGYLYCCGTCYYRFCCKKRHEKLDQRQCTNYQSPVWVQTPSTKVVSPGPENKYDPEKDKTNFTVYITCGVIAFVIVAGVFAKVSYDKAHRPPREMNIHRALADILRQQGPIPIAHCERETISAIDTSPKENTPVRSTSKNHYTPVRTAKQTPGDRQYNHPILSSATQTPTHEKPRMNNILTSATEPYDLSFSRSYQNLAHLPPSYESAVKTNPSKYSSLKRLTDKEADEYYMRRRHLPDLAARGTLPLNVIQMSQQKPLPRERPRRPIRAMSQDRVLSPRRGLPDEFGMPYDRILSDEQLLSTERLHSQDPLLSPERTAFPEQSLSRAISHTDVFVSTPVLDRYRMTKMHSHPSASNNSYATLGQSQTAAKRHAFASRRHNTVEQLHYIPGHHTCYTASKTEVTV

SEQ ID NO: 16 - protein, 908 aa, synthetic construct
MALRRLLLPPLLLSLLLSLASLHLPPGADAFSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVPISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATPGSGSGSARGRSGNRTLNAGAVGGRRAGGALARGGRELNSTARASGVPEAGSRRGQSAAAAAAAAAAASATVTYETCWGYYDVSGQYDKEFECNNSESGYLYCCGTCYYRFCCKKRHEKLDQRQCTNYQSPVWVQTPSTKVVSPGPENKYDPEKDKTNFTVYITCGVIAFVIVAGVFAKVSYDKAHRPPREMNIHRALADILRQQGPIPIAHCERETISAIDTSPKENTPVRSTSKNHYTPVRTAKQTPGDRQYNHPILSSATQTPTHEKPRMNNILTSATEPYDLSFSRSYQNLAHLPPSYESAVKTNPSKYSSLKRLTDKEADEYYMRRRHLPDLAARGTLPLNVIQMSQQKPLPRERPRRPIRAMSQDRVLSPRRGLPDEFGMPYDRILSDEQLLSTERLHSQDPLLSPERTAFPEQSLSRAISHTDVFVSTPVLDRYRMTKMHSHPSASNNSYATLGQSQTAAKRHAFASRRHNTVEQLHYIPGHHTCYTASKTEVTV

SEQ ID NO: 17 - protein, 917 aa, synthetic construct
MALRRLLLPPLLLSLLLSLASLHLPPGADAFSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVPISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATPGSGSGSARGRSGNRTLNAGAVGGRRAGGALARGGRELNSTARASGVPEAGSRRGQSAAAAAAAAAAASATVTYETCWGYYDVSGQYDKEFECNNSESGYLYCCGTCYYRFCCKKRHEKLDQRQCTNYQSPVWVQTPSTKVVSPGPENKYDPEKDKTNFTVYITCGVIAFVIVAGVFAKVSYDKAHRPPREMNIHRALADILRQQGPIPIAHCERETISAIDTSPKENTPVRSTSKNHYTPVRTAKQTPGDRQYNHPILSSATQTPTHEKPRMNNILTSATEPYDLSFSRSYQNLAHLPPSYESAVKTNPSKYSSLKRLTDKEADEYYMRRRHLPDLAARGTLPLNVIQMSQQKPLPRERPRRPIRAMSQDRVLSPRRGLPDEFGMPYDRILSDEQLLSTERLHSQDPLLSPERTAFPEQSLSRAISHTDVFVSTPVLDRYRMTKMHSHPSASNNSYATLGQSQTAAKRHAFASRRHNTVEQLHYIPGHHTCYTASKTEVTVYPYDVPDYA

SEQ ID NO: 18 - protein, 919 aa, synthetic construct
FSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVPISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATPGSGSGSRPSNDTSSVAPGPLPALLAHLRRLTGALAGGGSAAGTSANATKTSPASGTGAAARAPPPAELCHGYYDVMGQYDATFNCSTGSYRFCCGTCHYRFCCEHRHMRLAQASCSNYDTPRWATTPPPLAGGAGGAGGAGGGPGPGQAGWLEGGRAGGAGGRGGEGPGGSTAYVVCGVISFALAVGVGAKVAFSKASRAPRAHREINVPRALVDILRHQAGPATRPDRARSSSLTPGLGGPDSMAPRTPKNLYNTMKPSNLDNLHYNVNSPKHHAATLDWRAMPPPSPSLHYSTLSCSRSFHNLSHLPPSYEAAVKSELNRYSSLKRLAEKDLDEAYLKRRQLEMPRGTLPLHALRRPGTGGGYRMDGWGGPEELGLAPAPNPRRVMSQEHLLGDGSRASRYEFTLPRARLVSQEHLLLSSPEALRQSREHLLSPPRSPALPPDPTTRASLAASHSNLLLGPGGPPTPLHGLPPSGLHAHHHHALHGSPQPAWMSDAGGGGGTLARRPPFQRQGTLEQLQFIPGHHLPQHLRTASKNEVTV

SEQ ID NO: 19 - protein, 941 aa, synthetic construct
MPALLLLGTVALLASAAGPAGAFSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVPISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATPGSGSGSRPSNDTSSVAPGPLPALLAHLRRLTGALAGGGSAAGTSANATKTSPASGTGAAARAPPPAELCHGYYDVMGQYDATFNCSTGSYRFCCGTCHYRFCCEHRHMRLAQASCSNYDTPRWATTPPPLAGGAGGAGGAGGGPGPGQAGWLEGGRAGGAGGRGGEGPGGSTAYVVCGVISFALAVGVGAKVAFSKASRAPRAHREINVPRALVDILRHQAGPATRPDRARSSSLTPGLGGPDSMAPRTPKNLYNTMKPSNLDNLHYNVNSPKHHAATLDWRAMPPPSPSLHYSTLSCSRSFHNLSHLPPSYEAAVKSELNRYSSLKRLAEKDLDEAYLKRRQLEMPRGTLPLHALRRPGTGGGYRMDGWGGPEELGLAPAPNPRRVMSQEHLLGDGSRASRYEFTLPRARLVSQEHLLLSSPEALRQSREHLLSPPRSPALPPDPTTRASLAASHSNLLLGPGGPPTPLHGLPPSGLHAHHHHALHGSPQPAWMSDAGGGGGTLARRPPFQRQGTLEQLQFIPGHHLPQHLRTASKNEVTV

SEQ ID NO: 20 - protein, 950 aa, synthetic construct
MPALLLLGTVALLASAAGPAGAFSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVPISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATPGSGSGSRPSNDTSSVAPGPLPALLAHLRRLTGALAGGGSAAGTSANATKTSPASGTGAAARAPPPAELCHGYYDVMGQYDATFNCSTGSYRFCCGTCHYRFCCEHRHMRLAQASCSNYDTPRWATTPPPLAGGAGGAGGAGGGPGPGQAGWLEGGRAGGAGGRGGEGPGGSTAYVVCGVISFALAVGVGAKVAFSKASRAPRAHREINVPRALVDILRHQAGPATRPDRARSSSLTPGLGGPDSMAPRTPKNLYNTMKPSNLDNLHYNVNSPKHHAATLDWRAMPPPSPSLHYSTLSCSRSFHNLSHLPPSYEAAVKSELNRYSSLKRLAEKDLDEAYLKRRQLEMPRGTLPLHALRRPGTGGGYRMDGWGGPEELGLAPAPNPRRVMSQEHLLGDGSRASRYEFTLPRARLVSQEHLLLSSPEALRQSREHLLSPPRSPALPPDPTTRASLAASHSNLLLGPGGPPTPLHGLPPSGLHAHHHHALHGSPQPAWMSDAGGGGGTLARRPPFQRQGTLEQLQFIPGHHLPQHLRTASKNEVTVYPYDVPDYA

SEQ ID NO: 21 - DNA, 1047 bp, Mus musculus
  source 1..1047: mol_type = other DNA
gggggagtgcagaaggtggatgtgcctgccgcagaccttagcgaccaagtgccagacacagactctgagaccagaattatcctgcaagggagcccggtggttcagatggctgaagatgctgtggacggggagcggctgaaacacctgatcgtgacccccgcaggctgtggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagccgt

SEQ ID NO: 22 - DNA, 1131 bp, Mus musculus
  source 1..1131: mol_type = other DNA
ttcagcgggacccccgtgattagactacgcttcaagaggcttcagcctaccaggctgctggctgaatttgacttccgcacttttgaccctgaaggagtcctcttcttcgctggaggccgttcagacagcacctggattgtcctgggcctaagagctgggcggcttgagctgcagcttcggtacaatggcgttgggcgcatcaccagcagcgggccaaccatcaaccacggcatgtggcaaactatctccgtggaagagctggaacgtaaccttgtcatcaaggtcaacaaagatgctgtaatgaagatcgcggtagctggggagctgtttcagctggagaggggcctctatcacctgaatctcaccgtgggcggcattcccttcaaggagagtgagctcgtccagccgattaaccctcgcctggatgggtgcatgaggagttggaactggctgaacggggaagacagcgccatccaggagacagtcaaggcaaacacaaaaatgcagtgcttctctgtgacagaaaggggctccttcttcccggggaatggatttgctacctacaggctcaactacacccgaacatcgctggatgtcggcacggaaaccacctgggaagttaaagttgtggctcggatccgccctgccacggacacgggggtgctgctggcgctggtgggggacgacgatgtcgtccccatctctgtggccctagtcgactaccactctacaaagaagctcaagaagcagttggtggtcctggcagttgaggatgttgccctggcactgatggaaatcaaggtgtgcgacagccaggaacacacggtcactgtctccctgcgggagggtgaggccaccctagaagtggatggcacaaagggccagagtgaagtgagcactgcccagctgcaggagcgactggacacacttaagacacatctgcaaggctctgtgcacacctatgttggaggcctgccagaagtatcggtgatttctgcacccgtcactgcgttctaccgcggatgcatgactctggaggtaaacgggaaaatcctggacctggatacggcctcgtacaagcacagtgacatcacctcccactcctgcccgcctgtggagcatgccaccccc

SEQ ID NO: 23 - DNA, 1485 bp, Mus musculus
  source 1..1485: mol_type = other DNA
gcccgcggccgctccgggaaccggaccctgaacgcgggagccgtcggaggtcggcgtgctgggggcgccctggcccgaggcggccgcgaactaaacagcaccgcccgagcgtccggcgtcccggaggcgggcagccggcggggacagtccgcagcagcggcagcggcggcggcggcggcggccagcgcgactgttacttacgagacgtgctggggctactacgacgtgagcggccagtacgacaaggagttcgagtgcaacaacagcgagagcggctacctgtactgctgcggcacctgctactatcgcttctgctgcaagaagcgccacgagaagctggaccagcgccagtgcaccaactaccaaagccccgtatgggtgcagacgcccagcaccaaggtagtgtcgccggggcccgagaacaagtacgacccggagaaggacaagaccaacttcaccgtctacatcacttgcggggtgatcgccttcgtcatcgtggcgggcgtcttcgccaaggtctcctatgacaaggcccaccgccctccgcgagagatgaacatccacagggctctggctgacattctaagacaacagggaccaatccccatagcacactgtgaaagagaaaccatctcggccatcgatacctctcccaaagagaacacgccggtccgatcaacctccaaaaaccactacacccctgtgcgcacagccaagcagactccaggtgatcgtcagtataatcatccgatcttaagcagcgctacccagacccctacacatgagaagccacggatgaataacattctgacgtcggccacggaaccctatgacctctccttctcacgctcttaccagaacttagcccacttgcctccatcatatgaatctgcagtgaaaaccaatccaagcaagtactcgtctctgaagaggctaacggacaaggaagctgatgagtattacatgagaaggaggcacctgccagaccttgcagcccgtggtaccctccccctcaatgtcatccagatgtctcaacagaagccacttcctcgagaacggccacgcaggcccatcagggccatgtcccaggacagggtcttgtctccacgtcggggattgccagatgaattcggcatgccctatgaccgcatcttgtctgatgaacagctgctctccacagagcgcctgcactcccaggacccgttgctgtccccagagaggacagccttcccggagcagtcgctgtcgcgggccatctcgcatacggacgtctttgtgtccacgccagtgctggaccgctaccgcatgaccaagatgcactcccatcccagtgcctccaataactcctatgccaccctgggccagagccagacagcagccaagcgccacgcctttgcctctcgcagacacaacacggtggagcagttacactatatcccaggccatcacacctgctacacagccagcaagactgaagtgaccgtg

SEQ ID NO: 24 - DNA, 1608 bp, Mus musculus
  source 1..1608: mol_type = other DNA
cgcccatccaacgacacaagctcagtggccccgggcccgctgcccgcgctactcgcgcacctgcggcgcttgaccggggctctggcgggcggcgggagcgcggcaggtaccagcgccaacgccaccaagaccagccccgcgagtggcacgggcgcagcggcacgggcgcctcctccggccgagctctgccatggctactacgatgtcatgggccagtacgacgccaccttcaactgcagcaccggctcctaccgcttctgttgtggcacctgccactaccgtttctgctgcgagcaccgccacatgcgcctggcgcaggcctcctgctccaactacgacacgccacgctgggccaccacgcccccgccgctggctggaggcgccgggggcgctgggggtgcgggtgggggaccagggccgggccaggcagggtggctggaagggggccgggccgggggcgctgggggacgtgggggagagggcccagggggcagcacagcctacgtggtgtgcggagtcatcagtttcgccctggcggtgggcgtcggtgccaaagtggccttcagcaaggcgtcacgtgcacccagggcgcaccgggagatcaacgtgcccagagctctcgtggatattctcaggcatcaagcaggacctgcaacccgcccggaccgggccagaagcagttctctgaccccagggctgggaggcccagacagcatggccccaaggacacccaagaacctttacaacaccatgaagccctccaacctcgataacctgcactacaacgtcaacagccccaagcaccacgccgccacactggactggcgtgctatgccgccgcccagcccctccctgcactactccacactatcctgctcccgatccttccacaacctctctcatcttcccccgtcctatgaggctgctgtgaaatcagaactgaatcgatactcttccctcaagagactggctgagaaagatctggatgaagcctacctgaagcgcagacaactggagatgccgcgcggaacgctgcccttgcatgcactccggcggcctggcactggaggtggctaccgtatggatggctggggtggccctgaggagctgggcctggcaccagcacccaacccacggcgtgttatgtcccaggagcaccttctgggtgatggtagccgagcttcccgctatgagttcacgttgcctcgagcgcgcctggtgtctcaggaacacctgctgctgtcctcacctgaggcgcttcgccagagtcgcgagcacctgctgtcacccccacgaagtcctgcactgcccccagatcccaccacccgggccagcctggctgcctcacactccaacctgctgctagggcctgggggcccccccacacccctgcatgggttgcctccgtcaggcctgcatgcccaccatcaccatgcccttcatggctctcctcagccagcctggatgtctgatgcgggcgggggtgggggcacactggcccgcaggccacccttccagcgccagggcaccctggagcagctgcagttcattcctgggcaccacctgccccagcacctgcgcactgccagcaagaacgaagtgactgtc

SEQ ID NO: 25 - DNA, 90 bp, Mus musculus
  source 1..90: mol_type = other DNA
atggcgctgcgccgcctcctgctgccgcctctgctgctgtcgctgctgctgtcgctcgcgtccctgcacctgccgcccggcgcagacgcc

SEQ ID NO: 26 - DNA, 66 bp, Mus musculus
  source 1..66: mol_type = other DNA
atgccggccctgctgctgctcgggaccgtcgcgctgctagcctccgcagcgggcccggcgggggcg

SEQ ID NO: 27 - DNA, 18 bp, synthetic construct
  source 1..18: mol_type = other DNA
ggcagcggcagcggcagc

SEQ ID NO: 28 - DNA, 27 bp, synthetic construct
  source 1..27: mol_type = other DNA
tacccatacgatgttccagattacgct

SEQ ID NO: 29 - DNA, 2667 bp, synthetic construct
  source 1..2667: mol_type = other DNA
atggcgctgcgccgcctcctgctgccgcctctgctgctgtcgctgctgctgtcgctcgcgtccctgcacctgccgcccggcgcagacgccgggggagtgcagaaggtggatgtgcctgccgcagaccttagcgaccaagtgccagacacagactctgagaccagaattatcctgcaagggagcccggtggttcagatggctgaagatgctgtggacggggagcggctgaaacacctgatcgtgacccccgcaggctgtggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagccgtggcagcggcagcggcagcgcccgcggccgctccgggaaccggaccctgaacgcgggagccgtcggaggtcggcgtgctgggggcgccctggcccgaggcggccgcgaactaaacagcaccgcccgagcgtccggcgtcccggaggcgggcagccggcggggacagtccgcagcagcggcagcggcggcggcggcggcggccagcgcgactgttacttacgagacgtgctggggctactacgacgtgagcggccagtacgacaaggagttcgagtgcaacaacagcgagagcggctacctgtactgctgcggcacctgctactatcgcttctgctgcaagaagcgccacgagaagctggaccagcgccagtgcaccaactaccaaagccccgtatgggtgcagacgcccagcaccaaggtagtgtcgccggggcccgagaacaagtacgacccggagaaggacaagaccaacttcaccgtctacatcacttgcggggtgatcgccttcgtcatcgtggcgggcgtcttcgccaaggtctcctatgacaaggcccaccgccctccgcgagagatgaacatccacagggctctggctgacattctaagacaacagggaccaatccccatagcacactgtgaaagagaaaccatctcggccatcgatacctctcccaaagagaacacgccggtccgatcaacctccaaaaaccactacacccctgtgcgcacagccaagcagactccaggtgatcgtcagtataatcatccgatcttaagcagcgctacccagacccctacacatgagaagccacggatgaataacattctgacgtcggccacggaaccctatgacctctccttctcacgctcttaccagaacttagcccacttgcctccatcatatgaatctgcagtgaaaaccaatccaagcaagtactcgtctctgaagaggctaacggacaaggaagctgatgagtattacatgagaaggaggcacctgccagaccttgcagcccgtggtaccctccccctcaatgtcatccagatgtctcaacagaagccacttcctcgagaacggccacgcaggcccatcagggccatgtcccaggacagggtcttgtctccacgtcggggattgccagatgaattcggcatgccctatgaccgcatcttgtctgatgaacagctgctctccacagagcgcctgcactcccaggacccgttgctgtccccagagaggacagccttcccggagcagtcgctgtcgcgggccatctcgcatacggacgtctttgtgtccacgccagtgctggaccgctaccgcatgaccaagatgcactcccatcccagtgcctccaataactcctatgccaccctgggccagagccagacagcagccaagcgccacgcctttgcctctcgcagacacaacacggtggagcagttacactatatcccaggccatcacacctgctacacagccagcaagactgaagtgaccgtgtacccatacgatgttccagattacgct

SEQ ID NO: 30 - DNA, 2751 bp, synthetic construct
  source 1..2751: mol_type = other DNA
atggcgctgcgccgcctcctgctgccgcctctgctgctgtcgctgctgctgtcgctcgcgtccctgcacctgccgcccggcgcagacgccttcagcgggacccccgtgattagactacgcttcaagaggcttcagcctaccaggctgctggctgaatttgacttccgcacttttgaccctgaaggagtcctcttcttcgctggaggccgttcagacagcacctggattgtcctgggcctaagagctgggcggcttgagctgcagcttcggtacaatggcgttgggcgcatcaccagcagcgggccaaccatcaaccacggcatgtggcaaactatctccgtggaagagctggaacgtaaccttgtcatcaaggtcaacaaagatgctgtaatgaagatcgcggtagctggggagctgtttcagctggagaggggcctctatcacctgaatctcaccgtgggcggcattcccttcaaggagagtgagctcgtccagccgattaaccctcgcctggatgggtgcatgaggagttggaactggctgaacggggaagacagcgccatccaggagacagtcaaggcaaacacaaaaatgcagtgcttctctgtgacagaaaggggctccttcttcccggggaatggatttgctacctacaggctcaactacacccgaacatcgctggatgtcggcacggaaaccacctgggaagttaaagttgtggctcggatccgccctgccacggacacgggggtgctgctggcgctggtgggggacgacgatgtcgtccccatctctgtggccctagtcgactaccactctacaaagaagctcaagaagcagttggtggtcctggcagttgaggatgttgccctggcactgatggaaatcaaggtgtgcgacagccaggaacacacggtcactgtctccctgcgggagggtgaggccaccctagaagtggatggcacaaagggccagagtgaagtgagcactgcccagctgcaggagcgactggacacacttaagacacatctgcaaggctctgtgcacacctatgttggaggcctgccagaagtatcggtgatttctgcacccgtcactgcgttctaccgcggatgcatgactctggaggtaaacgggaaaatcctggacctggatacggcctcgtacaagcacagtgacatcacctcccactcctgcccgcctgtggagcatgccacccccggcagcggcagcggcagcgcccgcggccgctccgggaaccggaccctgaacgcgggagccgtcggaggtcggcgtgctgggggcgccctggcccgaggcggccgcgaactaaacagcaccgcccgagcgtccggcgtcccggaggcgggcagccggcggggacagtccgcagcagcggcagcggcggcggcggcggcggccagcgcgactgttacttacgagacgtgctggggctactacgacgtgagcggccagtacgacaaggagttcgagtgcaacaacagcgagagcggctacctgtactgctgcggcacctgctactatcgcttctgctgcaagaagcgccacgagaagctggaccagcgccagtgcaccaactaccaaagccccgtatgggtgcagacgcccagcaccaaggtagtgtcgccggggcccgagaacaagtacgacccggagaaggacaagaccaacttcaccgtctacatcacttgcggggtgatcgccttcgtcatcgtggcgggcgtcttcgccaaggtctcctatgacaaggcccaccgccctccgcgagagatgaacatccacagggctctggctgacattctaagacaacagggaccaatccccatagcacactgtgaaagagaaaccatctcggccatcgatacctctcccaaagagaacacgccggtccgatcaacctccaaaaaccactacacccctgtgcgcacagccaagcagactccaggtgatcgtcagtataatcatccgatcttaagcagcgctacccagacccctacacatgagaagccacggatgaataacattctgacgtcggccacggaaccctatgacctctccttctcacgctcttaccagaacttagcccacttgcctccatcatatgaatctgcagtgaaaaccaatccaagcaagtactcgtctctgaagaggctaacggacaaggaagctgatgagtattacatgagaaggaggcacctgccagaccttgcagcccgtggtaccctccccctcaatgtcatccagatgtctcaacagaagccacttcctcgagaacggccacgcaggcccatcagggccatgtcccaggacagggtcttgtctccacgtcggggattgccagatgaattcggcatgccctatgaccgcatcttgtctgatgaacagctgctctccacagagcgcctgcactcccaggacccgttgctgtccccagagaggacagccttcccggagcagtcgctgtcgcgggccatctcgcatacggacgtctttgtgtccacgccagtgctggaccgctaccgcatgaccaagatgcactcccatcccagtgcctccaataactcctatgccaccctgggccagagccagacagcagccaagcgccacgcctttgcctctcgcagacacaacacggtggagcagttacactatatcccaggccatcacacctgctacacagccagcaagactgaagtgaccgtgtacccatacgatgttccagattacgct

SEQ ID NO: 31 - DNA, 2766 bp, synthetic construct
  source 1..2766: mol_type = other DNA
atgccggccctgctgctgctcgggaccgtcgcgctgctagcctccgcagcgggcccggcgggggcggggggagtgcagaaggtggatgtgcctgccgcagaccttagcgaccaagtgccagacacagactctgagaccagaattatcctgcaagggagcccggtggttcagatggctgaagatgctgtggacggggagcggctgaaacacctgatcgtgacccccgcaggctgtggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagccgtggcagcggcagcggcagccgcccatccaacgacacaagctcagtggccccgggcccgctgcccgcgctactcgcgcacctgcggcgcttgaccggggctctggcgggcggcgggagcgcggcaggtaccagcgccaacgccaccaagaccagccccgcgagtggcacgggcgcagcggcacgggcgcctcctccggccgagctctgccatggctactacgatgtcatgggccagtacgacgccaccttcaactgcagcaccggctcctaccgcttctgttgtggcacctgccactaccgtttctgctgcgagcaccgccacatgcgcctggcgcaggcctcctgctccaactacgacacgccacgctgggccaccacgcccccgccgctggctggaggcgccgggggcgctgggggtgcgggtgggggaccagggccgggccaggcagggtggctggaagggggccgggccgggggcgctgggggacgtgggggagagggcccagggggcagcacagcctacgtggtgtgcggagtcatcagtttcgccctggcggtgggcgtcggtgccaaagtggccttcagcaaggcgtcacgtgcacccagggcgcaccgggagatcaacgtgcccagagctctcgtggatattctcaggcatcaagcaggacctgcaacccgcccggaccgggccagaagcagttctctgaccccagggctgggaggcccagacagcatggccccaaggacacccaagaacctttacaacaccatgaagccctccaacctcgataacctgcactacaacgtcaacagccccaagcaccacgccgccacactggactggcgtgctatgccgccgcccagcccctccctgcactactccacactatcctgctcccgatccttccacaacctctctcatcttcccccgtcctatgaggctgctgtgaaatcagaactgaatcgatactcttccctcaagagactggctgagaaagatctggatgaagcctacctgaagcgcagacaactggagatgccgcgcggaacgctgcccttgcatgcactccggcggcctggcactggaggtggctaccgtatggatggctggggtggccctgaggagctgggcctggcaccagcacccaacccacggcgtgttatgtcccaggagcaccttctgggtgatggtagccgagcttcccgctatgagttcacgttgcctcgagcgcgcctggtgtctcaggaacacctgctgctgtcctcacctgaggcgcttcgccagagtcgcgagcacctgctgtcacccccacgaagtcctgcactgcccccagatcccaccacccgggccagcctggctgcctcacactccaacctgctgctagggcctgggggcccccccacacccctgcatgggttgcctccgtcaggcctgcatgcccaccatcaccatgcccttcatggctctcctcagccagcctggatgtctgatgcgggcgggggtgggggcacactggcccgcaggccacccttccagcgccagggcaccctggagcagctgcagttcattcctgggcaccacctgccccagcacctgcgcactgccagcaagaacgaagtgactgtctacccatacgatgttccagattacgct

SEQ ID NO: 32 - DNA, 2850 bp, synthetic construct
  source 1..2850: mol_type = other DNA
atgccggccctgctgctgctcgggaccgtcgcgctgctagcctccgcagcgggcccggcgggggcgttcagcgggacccccgtgattagactacgcttcaagaggcttcagcctaccaggctgctggctgaatttgacttccgcacttttgaccctgaaggagtcctcttcttcgctggaggccgttcagacagcacctggattgtcctgggcctaagagctgggcggcttgagctgcagcttcggtacaatggcgttgggcgcatcaccagcagcgggccaaccatcaaccacggcatgtggcaaactatctccgtggaagagctggaacgtaaccttgtcatcaaggtcaacaaagatgctgtaatgaagatcgcggtagctggggagctgtttcagctggagaggggcctctatcacctgaatctcaccgtgggcggcattcccttcaaggagagtgagctcgtccagccgattaaccctcgcctggatgggtgcatgaggagttggaactggctgaacggggaagacagcgccatccaggagacagtcaaggcaaacacaaaaatgcagtgcttctctgtgacagaaaggggctccttcttcccggggaatggatttgctacctacaggctcaactacacccgaacatcgctggatgtcggcacggaaaccacctgggaagttaaagttgtggctcggatccgccctgccacggacacgggggtgctgctggcgctggtgggggacgacgatgtcgtccccatctctgtggccctagtcgactaccactctacaaagaagctcaagaagcagttggtggtcctggcagttgaggatgttgccctggcactgatggaaatcaaggtgtgcgacagccaggaacacacggtcactgtctccctgcgggagggtgaggccaccctagaagtggatggcacaaagggccagagtgaagtgagcactgcccagctgcaggagcgactggacacacttaagacacatctgcaaggctctgtgcacacctatgttggaggcctgccagaagtatcggtgatttctgcacccgtcactgcgttctaccgcggatgcatgactctggaggtaaacgggaaaatcctggacctggatacggcctcgtacaagcacagtgacatcacctcccactcctgcccgcctgtggagcatgccacccccggcagcggcagcggcagccgcccatccaacgacacaagctcagtggccccgggcccgctgcccgcgctactcgcgcacctgcggcgcttgaccggggctctggcgggcggcgggagcgcggcaggtaccagcgccaacgccaccaagaccagccccgcgagtggcacgggcgcagcggcacgggcgcctcctccggccgagctctgccatggctactacgatgtcatgggccagtacgacgccaccttcaactgcagcaccggctcctaccgcttctgttgtggcacctgccactaccgtttctgctgcgagcaccgccacatgcgcctggcgcaggcctcctgctccaactacgacacgccacgctgggccaccacgcccccgccgctggctggaggcgccgggggcgctgggggtgcgggtgggggaccagggccgggccaggcagggtggctggaagggggccgggccgggggcgctgggggacgtgggggagagggcccagggggcagcacagcctacgtggtgtgcggagtcatcagtttcgccctggcggtgggcgtcggtgccaaagtggccttcagcaaggcgtcacgtgcacccagggcgcaccgggagatcaacgtgcccagagctctcgtggatattctcaggcatcaagcaggacctgcaacccgcccggaccgggccagaagcagttctctgaccccagggctgggaggcccagacagcatggccccaaggacacccaagaacctttacaacaccatgaagccctccaacctcgataacctgcactacaacgtcaacagccccaagcaccacgccgccacactggactggcgtgctatgccgccgcccagcccctccctgcactactccacactatcctgctcccgatccttccacaacctctctcatcttcccccgtcctatgaggctgctgtgaaatcagaactgaatcgatactcttccctcaagagactggctgagaaagatctggatgaagcctacctgaagcgcagacaactggagatgccgcgcggaacgctgcccttgcatgcactccggcggcctggcactggaggtggctaccgtatggatggctggggtggccctgaggagctgggcctggcaccagcacccaacccacggcgtgttatgtcccaggagcaccttctgggtgatggtagccgagcttcccgctatgagttcacgttgcctcgagcgcgcctggtgtctcaggaacacctgctgctgtcctcacctgaggcgcttcgccagagtcgcgagcacctgctgtcacccccacgaagtcctgcactgcccccagatcccaccacccgggccagcctggctgcctcacactccaacctgctgctagggcctgggggcccccccacacccctgcatgggttgcctccgtcaggcctgcatgcccaccatcaccatgcccttcatggctctcctcagccagcctggatgtctgatgcgggcgggggtgggggcacactggcccgcaggccacccttccagcgccagggcaccctggagcagctgcagttcattcctgggcaccacctgccccagcacctgcgcactgccagcaagaacgaagtgactgtctacccatacgatgttccagattacgct

SEQ ID NO: 33 - DNA, 7166 bp, synthetic construct
  source 1..7166: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctataacttcgtataggatactttatacgaagttatcctcgagtaccggtcacgcgtacgcaagcttgataacttcgtatagcatacattatacgaagttatcctagggccacctcaagcgtaatctggaacatcgtatgggtacacggtcacttcagtcttgctggctgtgtagcaggtgtgatggcctgggatatagtgtaactgctccaccgtgttgtgtctgcgagaggcaaaggcgtggcgcttggctgctgtctggctctggcccagggtggcataggagttattggaggcactgggatgggagtgcatcttggtcatgcggtagcggtccagcactggcgtggacacaaagacgtccgtatgcgagatggcccgcgacagcgactgctccgggaaggctgtcctctctggggacagcaacgggtcctgggagtgcaggcgctctgtggagagcagctgttcatcagacaagatgcggtcatagggcatgccgaattcatctggcaatccccgacgtggagacaagaccctgtcctgggacatggccctgatgggcctgcgtggccgttctcgaggaagtggcttctgttgagacatctggatgacattgagggggagggtaccacgggctgcaaggtctggcaggtgcctccttctcatgtaatactcatcagcttccttgtccgttagcctcttcagagacgagtacttgcttggattggttttcactgcagattcatatgatggaggcaagtgggctaagttctggtaagagcgtgagaaggagaggtcatagggttccgtggccgacgtcagaatgttattcatccgtggcttctcatgtgtaggggtctgggtagcgctgcttaagatcggatgattatactgacgatcacctggagtctgcttggctgtgcgcacaggggtgtagtggtttttggaggttgatcggaccggcgtgttctctttgggagaggtatcgatggccgagatggtttctctttcacagtgtgctatggggattggtccctgttgtcttagaatgtcagccagagccctgtggatgttcatctctcgcggagggcggtgggccttgtcataggagaccttggcgaagacgcccgccacgatgacgaaggcgatcaccccgcaagtgatgtagacggtgaagttggtcttgtccttctccgggtcgtacttgttctcgggccccggcgacactaccttggtgctgggcgtctgcacccatacggggctttggtagttggtgcactggcgctggtccagcttctcgtggcgcttcttgcagcagaagcgatagtagcaggtgccgcagcagtacaggtagccgctctcgctgttgttgcactcgaactccttgtcgtactggccgctcacgtcgtagtagccccagcacgtctcgtaagtaacagtcgcgctggccgccgccgccgccgccgctgccgctgctgcggactgtccccgccggctgcccgcctccgggacgccggacgctcgggcggtgctgtttagttcgcggccgcctcgggccagggcgcccccagcacgccgacctccgacggctcccgcgttcagggtccggttcccggagcggccgcgggcgctgccgctgccgctgccacggctggggaggtggaaggacacatccatgttcaagtccttatggtcagggacatctgtttgatattgggccaaggcttggaataccatgaaggtagcctgggtggagccatagccgcctccgtagtatctttgctcattgagccagcgcactacagggggcacagagtcaaagtctttcagcagcagcagggccaggagggcgtaggatgtggcctctacgttgtagagctgctggtcaggctcctcccagcggttccgatctttggctgtgttcagaaacttgccgaggtaaggttcctccagtttgttcatcagggccagggcatacccagcaatggccactgtgtatggtctctgcaggttcatgtaactggcttcaatatactcccctgccttgttgatgctcccaggaaggctattgacctgcccctcacagatgtccctggcttcctgcagtgcgatgaggacgaaggctgtgagtgacacatctgcctccttggcgttccggaagccaccaatcatttcttggtgaatcacgggcccatcctcctgaaagacaccatccggcttctgtttctccagaatcaaccatttaacagccccacacaggacgtgagagtcgatggcgatgaggttggcagctagagagaagaccttgaccacgtaggctgtcagccaggtgctggggggccggttgttgaaggcagcataggcagagctgggctgtttgaaggccagctgctgggtgtaccctttcttgatgagctccagggcctcttgcctcttctctatgccgaacttctcccactgttcggtctggtccaggtagtgtaccgcaatgactgttggtgtcatgccaatcatgttctgttccccacagcctgcgggggtcacgatcaggtgtttcagccgctccccgtccacagcatcttcagccatctgaaccaccgggctcccttgcaggataattctggtctcagagtctgtgtctggcacttggtcgctaaggtctgcggcaggcacatccaccttctgcactcccccggcgtctgcgccgggcggcaggtgcagggacgcgagcgacagcagcagcgacagcagcagaggcggcagcaggaggcggcgcagcgccatggtggctctagaataacttcgtataaagtatcctatacgaagttataggtacctgagctcaacatcgatagcactagtataacttcgtataatgtatgctatacgaagttatacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 34 - DNA, 7250 bp, synthetic construct
  source 1..7250: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagcaagtttgtacaaaaaagcaggctataacttcgtataggatactttatacgaagttatcctcgagtaccggtcacgcgtacgcaagcttgataacttcgtatagcatacattatacgaagttatcctagggccacctcaagcgtaatctggaacatcgtatgggtacacggtcacttcagtcttgctggctgtgtagcaggtgtgatggcctgggatatagtgtaactgctccaccgtgttgtgtctgcgagaggcaaaggcgtggcgcttggctgctgtctggctctggcccagggtggcataggagttattggaggcactgggatgggagtgcatcttggtcatgcggtagcggtccagcactggcgtggacacaaagacgtccgtatgcgagatggcccgcgacagcgactgctccgggaaggctgtcctctctggggacagcaacgggtcctgggagtgcaggcgctctgtggagagcagctgttcatcagacaagatgcggtcatagggcatgccgaattcatctggcaatccccgacgtggagacaagaccctgtcctgggacatggccctgatgggcctgcgtggccgttctcgaggaagtggcttctgttgagacatctggatgacattgagggggagggtaccacgggctgcaaggtctggcaggtgcctccttctcatgtaatactcatcagcttccttgtccgttagcctcttcagagacgagtacttgcttggattggttttcactgcagattcatatgatggaggcaagtgggctaagttctggtaagagcgtgagaaggagaggtcatagggttccgtggccgacgtcagaatgttattcatccgtggcttctcatgtgtaggggtctgggtagcgctgcttaagatcggatgattatactgacgatcacctggagtctgcttggctgtgcgcacaggggtgtagtggtttttggaggttgatcggaccggcgtgttctctttgggagaggtatcgatggccgagatggtttctctttcacagtgtgctatggggattggtccctgttgtcttagaatgtcagccagagccctgtggatgttcatctctcgcggagggcggtgggccttgtcataggagaccttggcgaagacgcccgccacgatgacgaaggcgatcaccccgcaagtgatgtagacggtgaagttggtcttgtccttctccgggtcgtacttgttctcgggccccggcgacactaccttggtgctgggcgtctgcacccatacggggctttggtagttggtgcactggcgctggtccagcttctcgtggcgcttcttgcagcagaagcgatagtagcaggtgccgcagcagtacaggtagccgctctcgctgttgttgcactcgaactccttgtcgtactggccgctcacgtcgtagtagccccagcacgtctcgtaagtaacagtcgcgctggccgccgccgccgccgccgctgccgctgctgcggactgtccccgccggctgcccgcctccgggacgccggacgctcgggcggtgctgtttagttcgcggccgcctcgggccagggcgcccccagcacgccgacctccgacggctcccgcgttcagggtccggttcccggagcggccgcgggcgctgccgctgccgctgccgggggtggcatgctccacaggcgggcaggagtgggaggtgatgtcactgtgcttgtacgaggccgtatccaggtccaggattttcccgtttacctccagagtcatgcatccgcggtagaacgcagtgacgggtgcagaaatcaccgatacttctggcaggcctccaacataggtgtgcacagagccttgcagatgtgtcttaagtgtgtccagtcgctcctgcagctgggcagtgctcacttcactctggccctttgtgccatccacttctagggtggcctcaccctcccgcagggagacagtgaccgtgtgttcctggctgtcgcacaccttgatttccatcagtgccagggcaacatcctcaactgccaggaccaccaactgcttcttgagcttctttgtagagtggtagtcgactagggccacagagatggggacgacatcgtcgtcccccaccagcgccagcagcacccccgtgtccgtggcagggcggatccgagccacaactttaacttcccaggtggtttccgtgccgacatccagcgatgttcgggtgtagttgagcctgtaggtagcaaatccattccccgggaagaaggagcccctttctgtcacagagaagcactgcatttttgtgtttgccttgactgtctcctggatggcgctgtcttccccgttcagccagttccaactcctcatgcacccatccaggcgagggttaatcggctggacgagctcactctccttgaagggaatgccgcccacggtgagattcaggtgatagaggcccctctccagctgaaacagctccccagctaccgcgatcttcattacagcatctttgttgaccttgatgacaaggttacgttccagctcttccacggagatagtttgccacatgccgtggttgatggttggcccgctgctggtgatgcgcccaacgccattgtaccgaagctgcagctcaagccgcccagctcttaggcccaggacaatccaggtgctgtctgaacggcctccagcgaagaagaggactccttcagggtcaaaagtgcggaagtcaaattcagccagcagcctggtaggctgaagcctcttgaagcgtagtctaatcacgggggtcccgctgaaggcgtctgcgccgggcggcaggtgcagggacgcgagcgacagcagcagcgacagcagcagaggcggcagcaggaggcggcgcagcgccatggtggctctagaataacttcgtataaagtatcctatacgaagttataggtacctgagctcaacatcgatagcactagtataacttcgtataatgtatgctatacgaagttatacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 35 - protein, 1663 aa, Mus musculus
MGPASGSQLLVLLLLLASSPLALGIPMYSIITPNVLRLESEETIVLEAHDAQGDIPVTVTVQDFLKRQVLTSEKTVLTGASGHLRSVSIKIPASKEFNSDKEGHKYVTVVANFGETVVEKAVMVSFQSGYLFIQTDKTIYTPGSTVLYRIFTVDNNLLPVGKTVVILIETPDGIPVKRDILSSNNQHGILPLSWNIPELVNMGQWKIRAFYEHAPKQIFSAEFEVKEYVLPSFEVRVEPTETFYYIDDPNGLEVSIIAKFLYGKNVDGTAFVIFGVQDGDKKISLAHSLTRVVIEDGVGDAVLTRKVLMEGVRPSNADALVGKSLYVSVTVILHSGSDMVEAERSGIPIVTSPYQIHFTKTPKFFKPAMPFDLMVFVTNPDGSPASKVLVVTQGSNAKALTQDDGVAKLSINTPNSRQPLTITVRTKKDTLPESRQATKTMEAHPYSTMHNSNNYLHLSVSRMELKPGDNLNVNFHLRTDPGHEAKIRYYTYLVMNKGKLLKAGRQVREPGQDLVVLSLPITPEFIPSFRLVAYYTLIGASGQREVVADSVWVDVKDSCIGTLVVKGDPRDNHLAPGQQTTLRIEGNQGARVGLVAVDKGVFVLNKKNKLTQSKIWDVVEKADIGCTPGSGKNYAGVFMDAGLAFKTSQGLQTEQRADLECTKPAARRRRSVQLMERRMDKAGQYTDKGLRKCCEDGMRDIPMRYSCQRRARLITQGENCIKAFIDCCNHITKLREQHRRDHVLGLARSELEEDIIPEEDIISRSHFPQSWLWTIEELKEPEKNGISTKVMNIFLKDSITTWEILAVSLSDKKGICVADPYEIRVMQDFFIDLRLPYSVVRNEQVEIRAVLFNYREQEELKVRVELLHNPAFCSMATAKNRYFQTIKIPPKSSVAVPYVIVPLKIGQQEVEVKAAVFNHFISDGVKKTLKVVPEGMRINKTVAIHTLDPEKLGQGGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRSSATTFRLLWENGNLLRSEETKQNEAFSLTAKGKGRGTLSVVAVYHAKLKSKVTCKKFDLRVSIRPAPETAKKPEEAKNTMFLEICTKYLGDVDATMSILDISMMTGFAPDTKDLELLASGVDRYISKYEMNKAFSNKNTLIIYLEKISHTEEDCLTFKVHQYFNVGLIQPGSVKVYSYYNLEESCTRFYHPEKDDGMLSKLCHSEMCRCAEENCFMQQSQEKINLNVRLDKACEPGVDYVYKTELTNIELLDDFDEYTMTIQQVIKSGSDEVQAGQQRKFISHIKCRNALKLQKGKKYLMWGLSSDLWGEKPNTSYIIGKDTWVEHWPEAEECQDQKYQKQCEELGAFTESMVVYGCPN

SEQ ID NO: 36 - protein, 674 aa, Mus musculus
MPPPPGPAAALGTALLLLLLASESSHTVLLRAREAAQFLRPRQRRAYQVFEEAKQGHLERECVEEVCSKEEAREVFENDPETEYFYPRYQECMRKYGRPEEKNPDFAKCVQNLPDQCTPNPCDKKGTHICQDLMGNFFCVCTDGWGGRLCDKDVNECVQKNGGCSQVCHNKPGSFQCACHSGFSLASDGQTCQDIDECTDSDTCGDARCKNLPGSYSCLCDEGYTYSSKEKTCQDVDECQQDRCEQTCVNSPGSYTCHCDGRGGLKLSPDMDTCEDILPCVPFSMAKSVKSLYLGRMFSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVPISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATP

SEQ ID NO: 37 - DNA, 5139 bp, Mus musculus
  source 1..5139: mol_type = other DNA
agagaggagagccatataaagagccagcggctacagccccagctcgcctctgcccacccctgccccttaccccttcattccttccacctttttccttcactatgggaccagcttcagggtcccagctactagtgctactgctgctgttggccagctccccattagctctggggatccccatgtattccatcattactcccaatgtcctacggctggagagcgaagagaccatcgtactggaggcccacgatgctcagggtgacatcccagtcacagtcactgtgcaagacttcctaaagaggcaagtgctgaccagtgagaagacagtgttgacaggagccagtggacatctgagaagcgtctccatcaagattccagccagtaaggaattcaactcagataaggaggggcacaagtacgtgacagtggtggcaaacttcggggaaacggtggtggagaaagcagtgatggtaagcttccagagtgggtacctcttcatccagacagacaagaccatctacacccctggctccactgtcttatatcggatcttcactgtggacaacaacctactgcccgtgggcaagacagtcgtcatcctcattgagacccccgatggcattcctgtcaagagagacattctgtcttccaacaaccaacacggcatcttgcctttgtcttggaacattcctgaactggtcaacatggggcagtggaagatccgagccttttacgaacatgcgccgaagcagatcttctccgcagagtttgaggtgaaggaatacgtgctgcccagttttgaggtccgggtggagcccacagagacattttattacatcgatgacccaaatggcctggaagtttccatcatagccaagttcctgtacgggaaaaacgtggacgggacagccttcgtgatttttggggtccaggatggcgataagaagatttctctggcccactccctcacgcgcgtagtgattgaggatggtgtgggggatgcagtgctgacccggaaggtgctgatggagggggtacggccttccaacgccgacgccctggtggggaagtccctgtatgtctccgtcactgtcatcctgcactcaggtagtgacatggtagaggcagagcgcagtgggatcccgattgtcacttccccgtaccagatccacttcaccaagacacccaaattcttcaagccagccatgccctttgacctcatggtgttcgtgaccaaccccgatggctctccggccagcaaagtgctggtggtcactcagggatctaatgcaaaggctctcacccaagatgatggcgtggccaagctaagcatcaacacacccaacagccgccaacccctgaccatcacagtccgcaccaagaaggacactctcccagaatcacggcaggccaccaagacaatggaggcccatccctacagcactatgcacaactccaacaactacctacacttgtcagtgtcacgaatggagctcaagccgggggacaacctcaatgtcaacttccacctgcgcacagacccaggccatgaggccaagatccgatactacacctacctggttatgaacaaggggaagctcctgaaggcaggccgccaggttcgggagcctggccaggacctggtggtcttgtccctgcccatcactccagagtttattccttcatttcgcctggtggcttactacaccctgattggagctagtggccagagggaggtggtggctgactctgtgtgggtggatgtgaaggattcctgtattggcacgctggtggtgaagggtgacccaagagataaccatctcgcacctgggcaacaaacgacactcaggattgaaggaaaccagggggcccgagtggggctagtggctgtggacaagggagtgtttgtgctgaacaagaagaacaaactcacacagagcaagatctgggatgtggtagagaaggcagacattggctgcaccccaggcagtgggaagaactatgctggtgtcttcatggatgcaggcctggccttcaagacaagccaaggactgcagactgaacagagagcagatcttgagtgcaccaagccagcagcccgccgccgtcgctcagtacagttgatggaaagaaggatggacaaagctggtcagtacactgacaagggtcttcggaagtgttgtgaggatggtatgcgggatatccctatgagatacagctgccagcgccgggcacgcctcatcacccagggcgagaactgcataaaggccttcatagactgctgcaaccacatcaccaagctgcgtgaacaacacagaagagaccacgtgctgggcctggccaggagtgaattggaggaagacataattccagaagaagatattatctctagaagccacttcccacagagctggttgtggaccatagaagagttgaaagaaccagagaaaaatggaatctctacgaaggtcatgaacatctttctcaaagattccatcaccacctgggagattctggcagtgagcttgtcagacaagaaagggatctgtgtggcagacccctatgagatcagagtgatgcaggacttcttcattgacctgcggctgccctactctgtagtgcgcaacgaacaggtggagatcagagctgtgctcttcaactaccgtgaacaggaggaacttaaggtgagggtggaactgttgcataatccagccttctgcagcatggccaccgccaagaatcgctacttccagaccatcaaaatccctcccaagtcctcggtggctgtaccgtatgtcattgtccccttgaagatcggccaacaagaggtggaggtcaaggctgctgtcttcaatcacttcatcagtgatggtgtcaagaagacactgaaggtcgtgccagaaggaatgagaatcaacaaaactgtggccatccatacactggacccagagaagctcggtcaagggggagtgcagaaggtggatgtgcctgccgcagaccttagcgaccaagtgccagacacagactctgagaccagaattatcctgcaagggagcccggtggttcagatggctgaagatgctgtggacggggagcggctgaaacacctgatcgtgacccccgcaggctgtggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagccgtagctctgcaaccacgtttcgcctgctctgggaaaatggcaacctcctgcgatcggaagagaccaagcaaaatgaggccttctctctaacagccaaaggaaaaggccgaggcacattgtcggtggtggcagtgtatcatgccaaactcaaaagcaaagtcacctgcaagaagtttgacctcagggtcagcataagaccagcccctgagacagccaagaagcccgaggaagccaagaataccatgttccttgaaatctgcaccaagtacttgggagatgtggacgccactatgtccatcctggacatctccatgatgactggctttgctccagacacaaaggacctggaactgctggcctctggagtagatagatacatctccaagtacgagatgaacaaagccttctccaacaagaacaccctcatcatctacctagaaaagatttcacacaccgaagaagactgcctgaccttcaaagttcaccagtactttaatgtgggacttatccagcccgggtcggtcaaggtctactcctattacaacctcgaggaatcatgcacccggttctatcatccagagaaggacgatgggatgctcagcaagctgtgccacagtgaaatgtgccggtgtgctgaagagaactgcttcatgcaacagtcacaggagaagatcaacctgaatgtccggctagacaaggcttgtgagcccggagtcgactatgtgtacaagaccgagctaaccaacatagagctgttggatgattttgatgagtacaccatgaccatccagcaggtcatcaagtcaggctcagatgaggtgcaggcagggcagcaacgcaagttcatcagccacatcaagtgcagaaacgccctgaagctgcagaaagggaagaagtacctcatgtggggcctctcctctgacctctggggagaaaagcccaacaccagctacatcattgggaaggacacgtgggtggagcactggcctgaggcagaagaatgccaggatcagaagtaccagaaacagtgcgaagaacttggggcattcacagaatctatggtggtttatggttgtcccaactgactacagcccagccctctaataaagcttcagttgtatttcacccatc

SEQ ID NO: 38 - DNA, 2594 bp, Mus musculus
  source 1..2594: mol_type = other DNA
acccgctgcctccttcacggcccagaaccgcagcgagcgccgggtgcggcgctgccgccgcagggatgtgaccttcaccgtcgcttagccaggatgaccggagcccgtgtctcgcggcgtccgcgcctcgcttcagcctcccgggtgctctgaccgcacgctcccggctgctaggctccccggcaccggcctcgccatgccgccaccgcccgggcccgccgccgccctgggcactgcgcttctgctgctcctgctggcttccgagtcttctcacactgtgctgttgcgggcgcgtgaggcggcgcagtttctgcggcccaggcagcgccgcgcctaccaagtcttcgaggaggccaagcagggccacctggaacgggagtgcgtggaggaggtgtgcagcaaagaggaggccagagaggtgttcgagaacgaccccgagacggagtatttctatccacgatatcaagagtgcatgagaaaatatggcaggcctgaagaaaaaaacccagatttcgccaaatgtgttcagaacttgcctgaccagtgcaccccaaacccttgtgataagaagggtactcatatctgccaagacctcatgggcaacttcttctgcgtgtgcacagatggctggggaggccggctctgtgacaaagatgtcaatgagtgtgtccagaagaatgggggctgcagccaggtctgccacaacaaaccaggaagcttccaatgtgcctgccatagtggcttctcgcttgcatcagacggccagacctgccaagatatcgatgaatgcacagactcagacacctgtggggacgcgcgatgcaagaacttgccaggctcctactcttgcctctgcgatgagggatatacatacagctccaaggagaagacctgccaagatgtggacgagtgccagcaggatcgctgtgagcagacctgtgtcaactccccaggcagctatacctgccactgtgatgggcgagggggcctaaaactatccccagacatggatacttgtgaggacatcttaccatgtgtgcccttcagcatggccaagagcgtgaagtccttgtacctgggccgcatgttcagcgggacccccgtgattagactacgcttcaagaggcttcagcctaccaggctgctggctgaatttgacttccgcacttttgaccctgaaggagtcctcttcttcgctggaggccgttcagacagcacctggattgtcctgggcctaagagctgggcggcttgagctgcagcttcggtacaatggcgttgggcgcatcaccagcagcgggccaaccatcaaccacggcatgtggcaaactatctccgtggaagagctggaacgtaaccttgtcatcaaggtcaacaaagatgctgtaatgaagatcgcggtagctggggagctgtttcagctggagaggggcctctatcacctgaatctcaccgtgggcggcattcccttcaaggagagtgagctcgtccagccgattaaccctcgcctggatgggtgcatgaggagttggaactggctgaacggggaagacagcgccatccaggagacagtcaaggcaaacacaaaaatgcagtgcttctctgtgacagaaaggggctccttcttcccggggaatggatttgctacctacaggctcaactacacccgaacatcgctggatgtcggcacggaaaccacctgggaagttaaagttgtggctcggatccgccctgccacggacacgggggtgctgctggcgctggtgggggacgacgatgtcgtccccatctctgtggccctagtcgactaccactctacaaagaagctcaagaagcagttggtggtcctggcagttgaggatgttgccctggcactgatggaaatcaaggtgtgcgacagccaggaacacacggtcactgtctccctgcgggagggtgaggccaccctagaagtggatggcacaaagggccagagtgaagtgagcactgcccagctgcaggagcgactggacacacttaagacacatctgcaaggctctgtgcacacctatgttggaggcctgccagaagtatcggtgatttctgcacccgtcactgcgttctaccgcggatgcatgactctggaggtaaacgggaaaatcctggacctggatacggcctcgtacaagcacagtgacatcacctcccactcctgcccgcctgtggagcatgccaccccctagaccgagctgcaagagggctccacacctaaagacaaaaatgaagcagggtttggacacacagcactggctcctctcgcatggtcctgcaacactggagcaggtgggctaccgcccttgtggtttttttttcttgagatctttctttttgccttgtaacatatctgtacataatgggacgggtgtcgggtcaccggctgctcagagagagccacgtgacctggtgggagctggctggaaggggctgggctagaggggctggcagtttgcagcagaacggatgtgaagaaaataattctctattatttttattactacatgcttctttctgactctaaaatatggaaaataaaatatttacagaaacctttttaaa

(19) United States
(12) Patent Application Publication
(10) Pub. No.: US 2025/0368706 A1
(43) Pub. Date: Dec. 4, 2025

(54) SYNAPSE SURGERY TOOLS AND ASSOCIATED METHODS FOR NEURAL CIRCUIT-SPECIFIC SYNAPSE ABLATION AND MODIFICATION

(71) Applicant: Arizona Board of Regents on Behalf of the University of Arizona, Tucson, AZ (US)

(72) Inventors: Deveroux Ferguson, Tucson, AZ (US); Hee-Dae Kim, Tucson, AZ (US)

(21) Appl. No.: 18/861,165

(22) PCT Filed: Apr. 27, 2023
(86) PCT No.: PCT/US2023/066325

§ 371 Date: Oct. 28, 2024

Related U.S. Application Data
(60) Provisional application No. 63/335,613, filed on Apr. 27, 2022.

(51) Int. Cl. C07K 14/47; A61K 48/00; C07K 14/705

(52) U.S. Cl. CPC C07K 14/472; A61K 48/0058; C07K 14/47; C07K 14/705; C07K 2319/02; C07K 2319/42; C12N 2750/14143; C12N 2800/30; C12N 2830/008

(57) ABSTRACT

A synapse surgical tool for selectively removing or ablating a postsynaptic terminal from a neuron, the method comprising: delivering an expression vector comprising a nucleic acid encoding a fusion protein comprising an N-terminal domain comprising an activated glial receptor binding domain; and a C terminal domain comprising a transmembrane domain of postsynaptic protein; expressing the fusion protein so that the activated glial receptor binding domain is localized to a synaptic cleft of the postsynaptic terminal of the neuron; and contacting the neuron with an activated microglial cell so that the activated microglial binds to the activated glial receptor binding domain and selectively ablates the postsynaptic terminal of the neuron.

### CROSS-REFERENCE TO RELATED APPLICATIONS

This is a PCT application that claims benefit to U.S. Provisional Application Ser. No. 63/335,613 filed Apr. 27, 2022, which is herein incorporated by reference in its entirety.

### INCORPORATION BY REFERENCE OF SEQUENCE LISTING

This application contains a sequence listing that has been submitted via PatentCenter in a computer readable format and is hereby incorporated by reference in its entirety. The computer readable file, created on Aug. 5, 2025 is named 085067-752127_SequenceListing.xml and is 90,112 bytes in size.

### FIELD

The present disclosure generally relates to technologies associated with neuronal functions and connectivity of neurons, and in particular, to tools and associated methods for selective synapse elimination in specific neural circuits, or synapse surgery tools.

### BACKGROUND

The diverse connectivity of neurons is a fundamental source of various neuronal functions, including sensation, motor functions, memory formation, and consciousness. Proper connections of neurons are crucial for the optimal functions of the neural system. Synapses are the basic unit of information processing between neurons. During neurodevelopmental processes, appropriate connections are selected via axon guidance and pruning of excess synapses. Aberrant wiring of neurons during the developmental process is thought as the main cause of many neurological disorders such as autism spectrum disorders, epilepsy, dyslexia, etc. In the adult stage, brain connectivity is dynamically changed in normal and disease conditions. In terms of synaptic plasticity, specific synaptic connections are strengthened or weakened for the storage of information in learning and memory processes. On the other hand, in neuropathological conditions, misconnected or nonselective loss of synapses is a prerequisite condition of cognitive impairment in many neurodegenerative diseases. In other words, each neuron connects to others via 1000 synapses on average, and mis-wiring or loss of connection via the synapses is the underlying cause of many neurological diseases.

To test the functional relevance of each neural circuit, optogenetic and pharmacogenetic tools have been widely used to temporally control neural activities in circuit-specific manners. Although there have been the huge contributions of optogenetic tools for evaluating specific neural circuit functions, the current tools have limitations mainly originating from the optical approach itself, such as low-throughput, technical complexity, and procedural invasiveness. In addition, no existing tools can physically modify neural connections in a brain-wide manner, which is critical to resolving neural circuit-related disease conditions. Thus, it is crucial to have means to efficiently and structurally manipulate neural circuits in the era of whole-brain mapping.

It is with these observations in mind, among others, that various aspects of the present disclosure were conceived and developed.

### SUMMARY

The following presents a simplified summary of various aspects described herein. This summary is not an extensive overview and is not intended to identify key or critical elements or to delineate the scope of the claims. The following summary merely presents some concepts in a simplified form as an introductory prelude to the more detailed description provided below. Corresponding apparatus, methods/processes, systems, and computer-readable media are also within the scope of the disclosure.

The following presents a simplified summary of various aspects described herein. This summary is not an extensive overview and is not intended to identify key or critical elements or to delineate the scope of the claims. The following summary merely presents some concepts in a simplified form as an introductory prelude to the more detailed description provided below. Corresponding apparatus, methods/processes, systems, and computer-readable media are also within the scope of the disclosure.

In various aspects, a fusion protein is provided comprising: (a) an N terminal domain comprising an activated glial receptor binding domain; and (b) a C terminal domain comprising a transmembrane domain of a synaptic protein. In some aspects, (a) comprises an activated glial receptor binding domain of a C3 complement protein (e.g., C3dg peptide) a or an activated glial receptor binding domain of a Gas6 protein (e.g., Laminin-G like domain of Gas6).

In various aspects, an amino acid sequence of (a) has at least 70%, at least 75%, at least 80%, at least 85%, at least 90%, at least 95%, at least 96%, at least 97%, at least 98%, at least 99% or 100% sequence identity to SEQ ID NO: 1. In various aspects, an amino acid sequence of (a) comprises SEQ ID NO: 1.

In various aspects, amino acid sequence of (a) has at least 70%, at least 75%, at least 80%, at least 85%, at least 90%, at least 95%, at least 96%, at least 97%, at least 98%, at least 99% or 100% sequence identity to SEQ ID NO: 2. In some aspects, an amino acid sequence of (a) comprises SEQ ID NO: 2.

In various aspects, the synaptic protein of (b) is a postsynaptic protein (e.g., Shisa6 or Shisa7). In various aspects, an amino acid sequence of (b) has at least 70%, at least 75%, at least 80%, at least 85%, at least 90%, at least 95%, at least 96%, at least 97%, at least 98%, at least 99% or 100% sequence identity to SEQ ID NO: 3 or 4. In further aspects, an amino acid sequence of (b) comprises SEQ ID NO: 3 or 4.

In any of the foregoing or related aspects, a fusion protein herein may further comprise a signal peptide. In any of the foregoing or related aspects, a fusion protein herein may further comprise a peptide linker between (a) and (b). In any of the foregoing or related aspects, a fusion protein herein may further comprise an HA tag.

In any of the foregoing or related aspects, a fusion protein herein may comprise an amino acid sequence of any one of SEQ ID NOs: 9 to 20.

Also provided herein are nucleic acid encoding a fusion protein described herein and/or expression vectors comprising nucleic acids encoding a fusion protein described herein. In various aspects, the expression vectors provided herein may further comprise a neuron specific and/or Cre-dependent promoter operably linked to the nucleic acid encoding the fusion protein. In some aspects, the neuron specific and/or Cre-dependent promoter comprises a Syn1-DIO promoter, a Synapsin promoter, or a CamKIIa promoter. For example, the neuron specific and/or Cre-dependent promoter comprises a Syn1-DIO promoter. In various aspects, the vector comprises an AAV vector.

Further aspects of the present disclosure provide for a method of selectively removing or ablating a postsynaptic terminal from a neuron, the method comprising: (a) delivering an expression vector comprising a nucleic acid encoding a fusion protein (e.g., an expression vector provided herein) comprising: (i) an N terminal domain comprising an activated glial receptor binding domain; and (ii) a C terminal domain comprising a transmembrane domain of postsynaptic protein; (b) expressing the fusion protein so that the activated glial receptor binding domain is localized to a synaptic cleft of the postsynaptic terminal of the neuron; and (c) contacting the neuron with an activated microglial cell so that the activated microglial binds to the activated glial receptor binding domain and selectively ablates the postsynaptic terminal of the neuron.

In various aspects, the postsynaptic terminal may be an excitatory postsynaptic terminal. In various aspects, the postsynaptic terminal is an inhibitory postsynaptic terminal.

Also provided is a fusion protein system comprising a first fusion protein and a second fusion protein wherein: (a) the first fusion protein comprises an N terminal domain comprising a first fragment of an activated glial receptor binding domain (“the first fragment”) and a C terminal domain comprising a transmembrane domain of a presynaptic protein; and (b) the second fusion protein comprises an N terminal domain comprising a second fragment of an activated glial receptor binding domain (“the second fragment”) and a C terminal domain comprising a transmembrane domain of a postsynaptic protein; wherein the first and second fragment can associate to form a functional “activated glial receptor binding domain”.

In any of the fusion protein systems provided herein, the activated glial receptor binding domain comprises a C3dg peptide or a receptor binding laminin-G-like domain of Gas6. In any of the fusion protein systems provided herein, presynaptic protein can comprise synaptophysin. In any of the fusion protein systems provided herein, the postsynaptic protein comprises a Shisa6 or Shisa7 protein. In any of the fusion protein systems provided herein, the first fusion protein and/or the second fusion protein further comprise a signal peptide.

In any of the fusion protein systems provided herein, the first fusion protein and/or the second fusion protein further comprises a peptide linker or an HA tag.

Further aspects of the present disclosure provide for a set of nucleic acids comprising a first nucleic acid and second nucleic acid encoding the first fusion protein and the second fusion protein, respectively, of a fusion protein system provided herein. Also provided are a set of expression vectors comprising a first expression vector and a second expression vector comprising, respectively, the first nucleic acid and the second nucleic acid encoding the first fusion protein and the second fusion protein of the fusion protein system provided herein.

In various aspects, the first expression vector of the set of expression vectors may further comprise a neuron specific and/or Cre-dependent promoter operably linked to the first nucleic acid and/or the second expression vector further comprises a neuron specific and/or Cre-dependent promoter operably linked to the second nucleic acid. In various aspects, the neuron specific and/or Cre-dependent promoter comprises Syn1-DIO promoter, a Synapsin promoter, or a CamKIIa promoter. In any of these aspects, the first and/or second expression vector may comprise an AAV vector.

Also provided herein is a method of selectively removing or ablating a synaptic connection between a presynaptic neuron (a first neuron) and a postsynaptic neuron (a second neuron), the synaptic connection comprising a presynaptic terminal of the first neuron, a postsynaptic terminal of the second neuron, and a synaptic cleft between the postsynaptic terminal and the presynaptic terminal, the method comprising (a) delivering to the presynaptic neuron, a nucleic acid encoding a first fusion protein, where the first fusion protein comprises an N terminal domain comprising a first fragment of an activated glial receptor binding domain and a C terminal domain comprising a transmembrane domain of a presynaptic protein; (b) delivering to the postsynaptic neuron, a nucleic acid encoding a second fusion protein, where the second fusion protein comprises an N terminal domain comprising a second fragment of an activated glial receptor binding domain and a C terminal domain comprising a transmembrane domain of a postsynaptic protein; where the first fragment of (a) can associate with the second fragment of (b) to form a functional activated glial receptor binding domain; (c) expressing the first fusion protein in the presynaptic terminal of the first neuron such that the N terminus of the first fusion protein is localized to the synaptic cleft; (d) expressing the second fusion protein in the postsynaptic terminal of the second neuron such that the N terminus of the second fusion protein is localized to the synaptic cleft; (e) allowing the first fragment of an activated glial receptor binding domain of the first fusion protein complex with the second fragment of an activated glial receptor binding domain of the second fusion protein to form the functional activated glial receptor binding domain; and (f) contacting the synaptic connection with an activated microglial cell, wherein the activated microglial cell binds to the functional activated glial receptor binding domain, thereby selectively ablating the synaptic connection between the two neurons.

In any of these aspects the first fusion protein of (a) and the second fusion protein of (b) together comprise a fusion protein system provided herein. In various aspects, the first nucleic acid of (a) and the second nucleic acid of (c) are delivered using a set of expression vectors provided herein.

In any of the methods herein, the neuron(s) may be in vitro, in vivo, or in situ. In various aspects, the neuron(s) are human or mouse neuron(s).

Also provided are methods of correcting a synaptic disorder in a subject. In some aspects, the method comprising selectively removing or ablating a postsynaptic terminal in at least one neuron of a subject according to a method provided herein. In some aspects, the method comprises selectively removing or ablating a synaptic connection (synapse) in a brain of a subject according to a method provided herein.

In various aspects, the synaptic disorder comprises depression, anodynia, drug addiction, autism, epileptic seizure, schizophrenia, obsessive compulsive disorder, attention deficit hyperactivity disorder, or any combination thereof.

In various aspects, the subject is a human or is a mouse model of a human psychiatric disorder.

### BRIEF DESCRIPTION OF THE DRAWINGS

FIG. 1A-1C depict schematic diagrams of synaptic pruning events by complement system and glial cells in the brain. (1A) Schematic of diseased brain tissues with faulty connections (red). (1B, 1C) Conventional approaches to control faulty connections in diseased brain. Neuro-modulator drugs affect entire brain areas (1B) and surgical excision removes or damages surrounding normal tissues (1C). Both approaches may cause unwanted side-effects because e of the non-specificity. (1D) On the other hand, the molecular brain surgery tool only removes selected connections (synapses) marked with complement or Gas6 proteins.

FIGS. 2A-2C are graphical schematic diagrams of synaptic pruning events by complement system and glial cells in the brain including: 2A refinement mechanisms of neural connection in the developing visual system, 2B uncontrolled synaptic loss in Alzheimer's disease, and 2C a theoretical model of experimentally targeted synapse removal system.

FIGS. 3A-3B are graphical schematic diagrams of the synapse surgery tools described herein including: 3A illustration of using AAV for complement protein (C3) expression and glial cell activation, 3B excitatory or inhibitory synapse targeting using SHISH proteins (fluorescent proteins may be added as reporter tags, not shown), and 3C circuit-specific synapse targeting using protein fragment complementation strategy.

FIGS. 4A-4F are illustrations of the validation of synapse surgery tools at various levels of model systems. 4A shows schematic diagrams of synapse specific labeling of complement protein (Venus, a bright GFP variant; VN, venus N-term; VC, Venus C-term). 4B: Fluorescent complementation signals for synapse labeling were validated in NIH3T3 cell lines after co-expression of control constructs. 4C: Ex vivo brain slice preparation and confocal live imaging. 4D: Time-lapse imaging of hippocampal slices. Movement of a GFP labeled microglia (arrow; 10 min interval; from 2-day long live imaging by LSM 710 confocal system). We will image fluorescence protein-labeled complement and microglia simultaneously in this preparation to observe synapse ablation events in live imaging experiments. 4E: In vivo validation scheme and example experiments. Two different brain regions (A & B) will be injected with circuit-specific complement AAV pairs in each site. After removal of synapses, optogenetic activation will be applied. Validation of circuit functionality will be assessed by behavior tests and immunohistochemistry (using tissue clearing). 4F: Validation of tissue clearing and antibody staining (CLARITY) using mouse brain section. A brain/hydrogel block (3-mm thick) containing prefrontal cortex and anterior striatum regions were lipid-cleared and stained with MAP2 antibody to visualize neuronal processes (Imaged by LSM 710 confocal system).

FIGS. 5A-5C illustrate validation of specificity of synapse surgery tools in various neural circuit models. Circuit specificity of synapse surgery tools will be assessed in well characterized circuits of various brain regions (5A: striatum; 5B: hippocampus (DG, dentate gyrus); 5C: cortex (RSCg, granular retrosplenial cortex) of mouse brain. The eliminations of a specific circuit can be easily detected by assessing the distinct spatial patterns of synapses with fluorescence labeling (e.g. eGRASP). LEC, lateral entorhinal cortex; MEC, medial entorhinal cortex; ATN, anterior thalamic nucle; SUB, dorsal subiculum).

FIGS. 6A-6C illustrate designing an excitory synapse surgery tool using complement C3dg. 6A: The classical complement cascade. 6B: Designing an excitatory synapse targeting tool. The C3dg coding region is inserted into Shisa6 after the signal peptide. The C3dg-Shisa6 fusion construct is subcloned in a Cre-dependent AAV vector, driven by a neuron specific promoter (AAV-Syn1-DIO). TM, transmembrane domain. 6C: Schematics of working model.

FIG. 7A-7C are representative images depicting activation of microglia by AAV vectors in vivo and in vitro. (A) A series of images illustrating activation of microglia upon AAV injection in adult mouse brain. AAV injection significantly increases activated microglia (IBA1+) in both NAc and images of Engulfment of C3dg tagged spines in adult mouse brain. Scale bar, 200 μm. Ac, anterior commissure. (B) Mixed primary glial culture from newborn pups (P1 neonates; DIV 7). Microglia grow on the astrocytic layer. (C) Microglia were isolated from mixed culture by flask tapping. Initially isolated microglia are mostly ramified forms (quiescent) (upper). Activated glia (ameboid) were markedly increased 24 hr after AAV treatment (control virus, 2×109 GC/ml). Scale bar, 100 μm.

FIGS. 8A-8B are a series of images illustrating activation of microglia upon AAV injection in adult mouse brain. AAV injection significantly increases activated microglia (IBA1+) in both NAc and images of Engulfment of C3dg tagged spines in adult mouse brain. Colocalization of activated microglia (IBA1+; blue) and HA tagged C3dg signals (green) in the NAc (8A) and hippocampus (8B) (inset; arrow).

FIGS. 9A-9B are images illustrating structural modifications in C3dg expressing neurons. Representative images of C3dg-negative (9A) and positive (9B) neurons in the NAc of Shisa6-C3dg AAV injected mice. Spine density is markedly reduced in C3dg expressing neurons (HA+) in the NAc (arrows and inset images). Scale bar, 20 μm.

FIGS. 10A-10B are illustrations related to the validation of functional synapse removal using monosynaptic retrograde tracing. 10A: Schematics of viral injection. 10B: The number mPFC neurons, projecting to D1 neurons in the NAc, markedly reduced in the C3dg injected group, while TVA expressing, rabies virus receptive neurons are abundant in the NAc of both groups (sagittal brain sections: NAc: 0.96 mm, mPFC: 0.24 mm lateral). Scale bar, 200 μm.

FIGS. 11A-11D are graphs illustrating behavioral assessments of C3dg expression in D1-neurons. Anxiety-(A, B; open field) and anhedonia-like behaviors (C, D; sucrose preference) were tested in control (DIO-mCherry), Shisa6 (DIO-Shisa6) and C3dg (DIO-Shisa6-C3dg) AAV injected mice. Shisa6 expression in D1-neurons increases anxiety (11A) and anhedonia (11C), whereas the behavioral phenotypes were reversed in C3dg injected mice (11B, 11D). Data are represented as mean±s.e.m. (*p<0.05); n=8-10 for each group; significant outliers were removed by Grubbs' test (alpha=0.05).

FIGS. 12A-12B are images illustrating the designing of circuit specific tools using fragment complementation strategy. (12A) The binding motif of C3 (C3dg) contains an aspartate (Asp) residue for CR3 binding. Simvastatin and mAB 107 antagonize the integration. (12B) Serial fragmentation of C3dg. The position of binding motif and in silico structural prediction will be considered to choose putative protein fragmentation pairs of C3dg.

FIGS. 13A-13B are images illustrating depression relevant-neural circuits in a preclinical model of depression. Each afferent input specific synapse were labeled using the dual-eGRASP technique. (13A) Schematic diagrams of D1-MSN specific eGRASP labeling in the NAc. mPFC afferent synapses labeled with cyan, whereas vHIP afferent synapses labeled with yellow fluorescent protein. (13B) Identification of eGRASP signals in the NAc coronal sections. This D1-dendrite in the NAc shows dense afferent innervations from vHIP region, which convey excitatory inputs inducing depression-like behaviors, relative to the mPFC. Ac, anterior commissure. Scale bar, 10 μm.

FIGS. 14A-14B depict a schematic (FIG. 14A) and working model (FIG. 14B) for the designing of an excitory synapse surgery tool using the Gas6. (14A) The coding region of LG1/LG2 domains of Gas6 is inserted into Shisa6 after the signal peptide. The Gas6-Shisa6 fusion construct is subcloned in a Cre-dependent AAV vector, driven by a neuron specific promoter (AAV-Syn1-DIO). TM, transmembrane domain. 14B: Schematics of working model.

FIG. 15 is a schematic of an exemplary expression plasmid (AAV-DIO-C3dg-Shisa6) according to various aspects of the present disclosure.

FIG. 16 is a schematic of an exemplary expression plasmid (AAV-DIO-Gas6-Shisa6) according to various aspects of the present disclosure.

Corresponding reference characters indicate corresponding elements among the view of the drawings. The headings used in the figures do not limit the scope of the claims.

### DETAILED DESCRIPTION

Various embodiments herein relate to an inventive concept associated with synapse surgery tools for selective synapse modification in specific neural circuits and associated methods and/or systems as described. The synapse surgery tools may use neuroimmune mechanisms and are highly specific for target synapses which are crucial for studying pathophysiological mechanisms of diverse neuronal disease with malfunctioning neural circuits. Furthermore, targeted modifications of specific neural circuits implicate novel gene therapy tools for neurological diseases.

The present inventive concept is inspired at least in part by the need for synapse manipulations and tools. Currently, optical approaches such as optogenetics or optical manipulations (photoablation) while imaging neural circuits are available to remove specific synapses. Although the optical approaches ensure precise ablation of target synapses, limitations which come from the use of imaging approach itself have hindered general use of the methods in wide neuroscience field: low-throughput such as limited accessibility of target areas (mostly cortical regions with cranial window) and a limited number of targetable synapses, invasiveness of the technique, and special equipment requirements. Thus, more simple approaches are needed to structurally modify neural connections on a brain-wide scale.

Growing evidence shows that neuron-glia interactions are crucial to refining synaptic connections. Recently, it has been reported that glial cells are intimately involved in neuronal connectivity at the level of synapse formation and pruning. Stevens and colleagues revealed that the classical complement system in the brain is crucial to synaptic pruning mechanisms. Complement proteins like C1q and C3 mark synapses for elimination. The complement system is not only involved in developmental processes but also observed in neurodegenerative diseases like Alzheimer's disease which leads to uncontrolled loss of synapses.

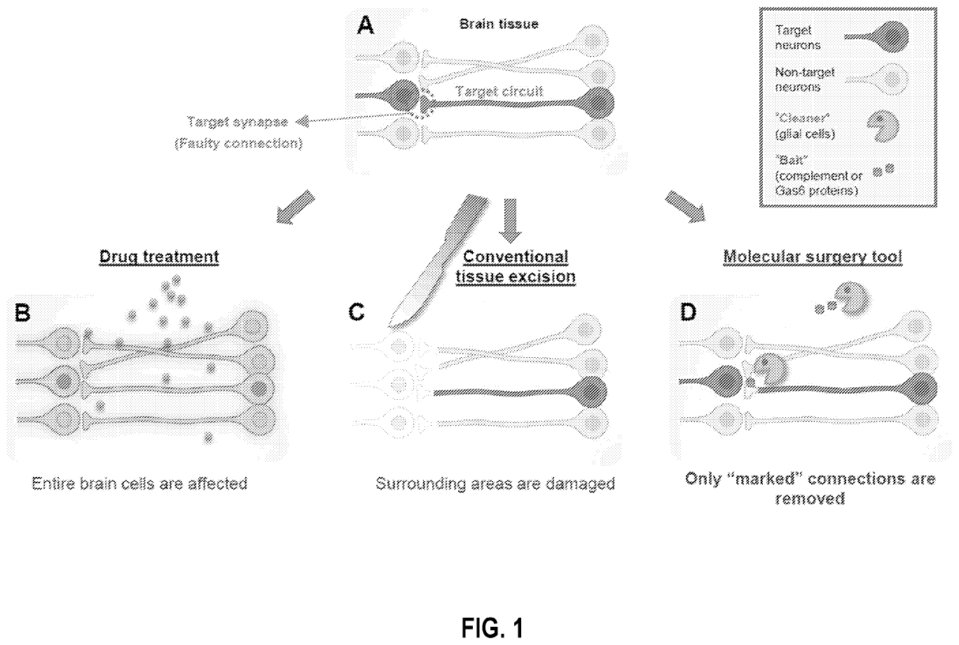

The present disclosure harnesses the endogenous complement system to remove selected synapses to study neural circuits. In essence, the disclosure describes compositions and methods of tagging desired synapses with specific complement protein(s) or other proteins that bind to activated glial cells and then using locally activated glial cells to remove the target synapses by the endogenous synapse pruning mechanism (FIG. 1). These tools not only can be used to interrogate neural circuits in more precise ways but also enable to cure of neurological diseases related to abnormal neural connections. Accordingly, the following description relates to tools for selective synapse elimination in specific neural circuits, so-called synapse surgery tools.

### I. Compositions

### (a) Single Fusion Proteins

Various aspects of the present disclosure are related to fusion proteins. In various aspects, the fusion proteins comprise at least two domains, optionally connected with a peptide linker. The fusion proteins can, in some aspects, comprise a protein or fragment thereof that can bind to a receptor on the surface of an activated glial cell as one domain (e.g., an N terminal domain). As used herein, a domain that can bind to a receptor on the surface of an activated glial cell is referred to as an “activated glial receptor binding domain.” The fusion proteins can also, in some aspects comprise a transmembrane domain of a synaptic protein (e.g., a postsynaptic or presynaptic protein) as the second domain (e.g., a C terminal domain). Together, the fusion proteins allow for the localization of the N terminus (containing the activated glial receptor binding domain) to a synaptic cleft. As is understood in the art, a synaptic cleft is a space between a presynaptic terminal and a postsynaptic terminal of a neuronal synapse where synaptic transmission takes place.

Various proteins contain activated glial receptor binding domains and may be used in aspects of the present disclosure. In various aspects, the activated glial receptor binding domain may be derived from a complement protein or a Gas6 protein, which are described herein below.

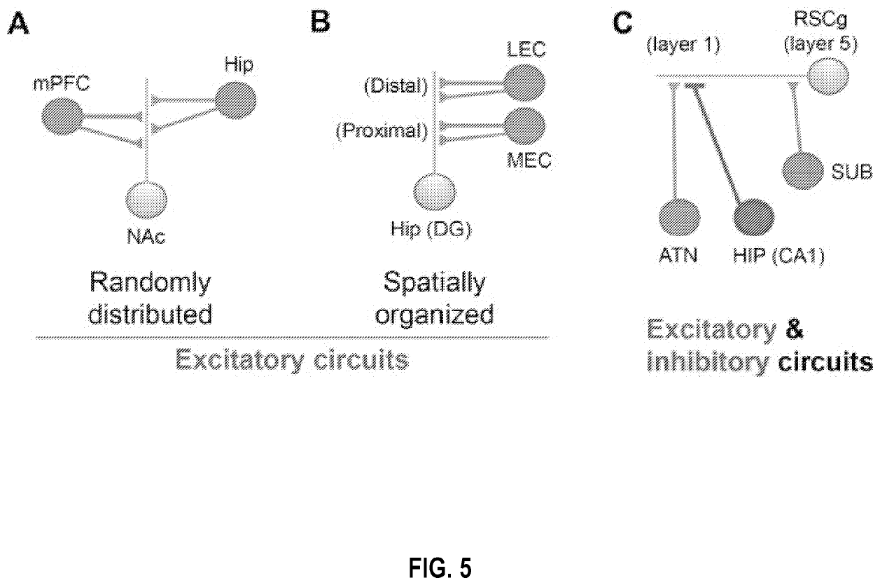

The complement system is an important component of the immune system, with the capacity to amplify immune responses by acting as a bridge between the innate and adaptive immune systems. When activated, complement proteins can promote inflammation by recruiting immune cells to the site of injury or infection and triggering the release of pro-inflammatory cytokines. In the context of microglial activation, the complement system can have both pro-inflammatory and protective effects, depending on the specific situation and signaling pathways involved. For instance, in neurodegenerative diseases like Alzheimer's, complement activation has been shown to contribute to inflammation and neuronal damage. However, in other contexts, such as during the removal of dying cells, complement activation may facilitate the clearance of cellular debris and help resolve inflammation. One exemplary complement protein that may be employed in the compositions herein is complement C3. Full length C3 is a 1663 amino acid protein (SEQ ID NO: 35). As shown in FIG. 5A, C3 is involved in a classic complement cascade which begins by activing C3 to leave it into two fragments: C3a and C3b. C3b is further cleaved by factor I into iC3b and finally to C3dg and C3c. It is C3dg (e.g., residues 955-1303 of SEQ ID NO: 35) that binds specifically to the complement receptor expressed by activated microglia (e.g., CR3 receptor) and therefore is an activated glial binding domain usable in the fusion proteins disclosed herein. As such, the C3dg domain is provided herein as SEQ ID NO: 1.

The Gas6-TAM signaling pathway plays a vital role in the regulation of the immune response, particularly in the context of phagocytosis and the resolution of inflammation. Gas6 signaling through TAM receptors on microglia has been shown to suppress the release of pro-inflammatory cytokines and promote the expression of anti-inflammatory mediators, such as interleukin-10 (IL-10). As used herein, the term “TAM receptors” or “TAM receptor” refers to a receptor that is a member of the TAM receptor family which includes Tyro3, Axl, and Mer receptors. Gas6-TAM signaling has been associated with the “M2” phenotype of microglia, which is characterized by a pro-resolving and anti-inflammatory profile. In this context, Gas6-TAM signaling can be considered to have mainly anti-inflammatory or immune-regulatory effects, contributing to the maintenance of CNS homeostasis. This property may counterbalance the pro-inflammatory effects of complement system activation. Specific domains of Gas6, laminin G-like domains (LG1/LG2 domains), have been used to successfully used to specifically remove amyloid plaque from an Alzheimer's disease model without apparent off-target and pro-inflammatory effects (Jung et al., 2022, Nat Medicine 28:1802). The full length protein for Gas6 is 674 amino acids and is provided herein as SEQ ID NO: 36. The L1/L2 domains found to bind to TAM receptors comprise residues 298-674 of the full length protein and are provided herein as SEQ ID NO: 2.

In accord with the foregoing, the fusion proteins provided herein may comprise a complement protein, peptide, or activated glial binding domain thereof. In some aspects, the fusion protein comprises a complement C3 peptide or activated glial binding domain thereof. In some aspects, the fusion protein comprises a C3dg peptide.

In various aspects, the fusion protein can comprise a domain (e.g., an N domain) comprising an amino acid sequence having at least 70%, at least 75%, at least 80%, at least 85%, at least 90%, at least 95%, at least 96%, at least 97%, at least 98%, at least 99% or 100% sequence identity with SEQ ID NO: 1. For example, in some aspects the fusion protein comprises an N terminal domain comprising an amino acid sequence comprising or consisting of SEQ ID NO: 1.

In further aspects, the fusion proteins provided herein may comprise a Gas6 peptide or activated glial binding domain thereof. In various aspects, the Gas6 peptide comprises a lamin-G like domain (e.g., a LG1 and/or an LG2 domain). These domains are known to specifically bind to a TAM receptor on activated glial cells.

In various aspects, the fusion protein can comprise a domain (e.g., an N domain) comprising an amino acid sequence having at least 70%, at least 75%, at least 80%, at least 85%, at least 90%, at least 95%, at least 96%, at least 97%, at least 98%, at least 99% or 100% sequence identity with SEQ ID NO: 2. For example, in some aspects the fusion protein comprises an N terminal domain comprising an amino acid sequence comprising or consisting of SEQ ID NO: 2.

As noted, the C-terminal domain of fusion proteins provided herein may comprise a transmembrane domain of a synaptic protein (e.g., a postsynaptic or presynaptic protein). In various aspects the synaptic protein may be a postsynaptic protein to enable localization of the fusion protein to a postsynaptic terminal of a target neuron. Exemplary postsynaptic proteins that may be used herein include Shisa6 and Shisa7, which are expressed exclusively in excitatory and inhibitory post synaptic terminals, respectively. In some aspects, the fusion protein comprises a full Shisa6 or Shisa7 protein and the N-terminal domain described above is connected to the N terminus (including, in some aspects, after the signal peptide) of the Shisa6 or Shisa7 protein.

Accordingly, in various aspects, the fusion proteins may comprise a transmembrane domain derived from a Shisa6 protein. In some aspects, the transmembrane domain of the Shisa6 protein comprises an amino acid sequence having at least 70%, at least 75%, at least 80%, at least 85%, at least 90%, at least 95%, at least 96%, at least 97%, at least 98%, at least 99% or 100% sequence identity to SEQ ID NO: 3. In some aspects, the transmembrane domain of the Shisa6 protein comprises an amino acid sequence comprising SEQ ID NO: 3. As mentioned, in some aspects, the fusion protein may comprise a full Shisa6 protein.

Accordingly, in various aspects, the fusion proteins may comprise a transmembrane domain derived from a Shisa7 protein. In some aspects, the transmembrane domain of the Shisa7 protein comprises an amino acid sequence having at least 70%, at least 75%, at least 80%, at least 85%, at least 90%, at least 95%, at least 96%, at least 97%, at least 98%, at least 99% or 100% sequence identity to SEQ ID NO: 4. In some aspects, the transmembrane domain of the Shisa7 protein comprises an amino acid sequence comprising SEQ ID NO: 4. As mentioned, in some aspects, the fusion protein may comprise a full Shisa7 protein.

In accord with the foregoing, the fusion proteins having the domains described may comprise from their N terminus to their C terminus: the activated glial receptor binding domain and the transmembrane domain of the synaptic protein. In some aspects, the fusion proteins further comprise a peptide linker (e.g., that connects the activated glial receptor binding domain and the transmembrane domain of the synaptic protein). Suitable linkers are known in the art and are typically chosen to be flexible, to allow for greatest freedom of motion for the activated glial receptor binding domain. In some aspects, the linker can comprise GSGSGS (SEQ ID NO: 5).

In further aspects, the fusion proteins described herein may further comprise a signal peptide at their N terminus. In view of the description above, fusion proteins containing the signal peptide, therefore, comprise, from their N terminus to their C terminus at least: the signal peptide, the activated glial receptor binding domain, an optional linker and the transmembrane domain of the synaptic protein. In some aspects, the signal peptide may be derived from the transmembrane synaptic protein (e.g., Shisa6 or Shisa7). For example, in some aspects, the signal peptide may a signal peptide of Shisa6 (e.g., MALRRLLLPPLLLSLLLSLASLHLPPGADA, SEQ ID NO: 6) or Shisa7 (e.g., MPALLLLGTVALLASAAGPAGA, SEQ ID NO: 7).

The fusion proteins may further comprise an HA tag (e.g., at a C-terminus). Suitable HA tags are known in the art. In some aspects, the fusion protein may comprise an HA tag comprising YPYDVPDYA (SEQ ID NO: 8).

The fusion proteins may further comprise a fluorophore or fragment thereof (e.g., at their N terminus) to allow for visualization of the synapse when expressed. This aspect is described in more detail below in the context of fusion protein systems for whole circuit analysis (e.g., see Sections (I) (b) and (II) (e), below).

Exemplary fusion proteins in accord with aspects of this disclosure are described in the Table below. In various aspects, the fusion protein provided herein may comprise an amino acid sequence having at least 70%, at least 75%, at least 80%, at least 85%, at least 90%, at least 95%, at least 96%, at least 97%, at least 98%, at least 99% or 100% sequence identity to any one of SEQ ID NOs: 9-20. In some aspects, the fusion protein has an amino acid sequence comprising any one of SEQ ID NOs: 9-20. In some aspects, the fusion protein has an amino acid sequence comprising any one of SEQ ID NOs: 9, 12, 15, and 18. For example, the fusion protein may have an amino acid sequence comprising SEQ ID NO: 9. For example, the fusion protein may have an amino acid sequence comprising SEQ ID NO: 12. For example, the fusion protein may have an amino acid sequence comprising SEQ ID NO: 15. For example, the fusion protein may have an amino acid sequence comprising SEQ ID NO: 18. In some aspects, the fusion protein has an amino acid sequence comprising any one of SEQ ID NOs: 10, 13, 16, and 19. For example, the fusion protein may have an amino acid sequence comprising SEQ ID NO: 10. For example, the fusion protein may have an amino acid sequence comprising SEQ ID NO: 13. For example, the fusion protein may have an amino acid sequence comprising SEQ ID NO: 16. For example, the fusion protein may have an amino acid sequence comprising SEQ ID NO: 19. In some aspects, the fusion protein has an amino acid sequence comprising any one of SEQ ID NOs: 11, 14, 17, and 20. For example, the fusion protein may have an amino acid sequence comprising SEQ ID NO: 11. For example, the fusion protein may have an amino acid sequence comprising SEQ ID NO: 14. For example, the fusion protein may have an amino acid sequence comprising SEQ ID NO: 17. For example, the fusion protein may have an amino acid sequence comprising SEQ ID NO: 20. For ease of reference, SEQ ID NOs: 9-20 are provided in an annotated form in Table 1 below.

TABLE 1
Illustrative Fusion Proteins

| | | | SEQ |
|---|---|---|---|
| Target | Sequence | | ID |
| Synapse | Description | Sequence (annotated) | NO: |
| Excitatory | C3dg-Shisa6 | GGVQKVDVPAADLSDQVPDTDSETRIILQGSPV | 9 |
| | Protein | VQMAEDAVDGERLKHLIVTPAGCGEQNMIGMT | |
| | linker, | PTVIAVHYLDQTEQWEKFGIEKRQEALELIKKGY | |
| | without | TQQLAFKQPSSAYAAFNNRPPSTWLTAYVVKVF | |
| | signal | SLAANLIAIDSHVLCGAVKWLILEKQKPDGVFQE | |
| | peptide or | DGPVIHQEMIGGFRNAKEADVSLTAFVLIALQEA | |
| | HA tag | RDICEGQVNSLPGSINKAGEYIEASYMNLQRPYT | |
| | (linker | VAIAGYALALMNKLEEPYLGKFLNTAKDRNRWE | |
| | underlined | EPDQQLYNVEATSYALLALLLLKDFDSVPPVVR | |
| | and | WLNEQRYYGGGYGSTQATFMVFQALAQYQTD | |
| | italicized) | VPDHKDLNMDVSFHLPSRGSGSGSARGRSGN | |
| | | RTLNAGAVGGRRAGGALARGGRELNSTARASG | |
| | | VPEAGSRRGQSAAAAAAAAAAASATVTYETCW | |
| | | GYYDVSGQYDKEFECNNSESGYLYCCGTCYYR | |
| | | FCCKKRHEKLDQRQCTNYQSPVWVQTPSTKVV | |
| | | SPGPENKYDPEKDKTNFTVYITCGVIAFVIVAGV | |
| | | FAKVSYDKAHRPPREMNIHRALADILRQQGPIPI | |
| | | AHCERETISAIDTSPKENTPVRSTSKNHYTPVRT | |
| | | AKQTPGDRQYNHPILSSATQTPTHEKPRMNNIL | |
| | | TSATEPYDLSFSRSYQNLAHLPPSYESAVKTNP | |
| | | SKYSSLKRLTDKEADEYYMRRRHLPDLAARGTL | |
| | | PLNVIQMSQQKPLPRERPRRPIRAMSQDRVLSP | |
| | | RRGLPDEFGMPYDRILSDEQLLSTERLHSQDPL | |
| | | LSPERTAFPEQSLSRAISHTDVFVSTPVLDRYRM | |
| | | TKMHSHPSASNNSYATLGQSQTAAKRHAFASR | |
| | | RHNTVEQLHYIPGHHTCYTASKTEVTV | |
| Excitatory | C3dg-Shisa6 | MALRRLLLPPLLLSLLLSLASLHLPPGADAGGV | 10 |
| | protein with | QKVDVPAADLSDQVPDTDSETRIILQGSPVVQM | |
| | signal | AEDAVDGERLKHLIVTPAGCGEQNMIGMTPTVIA | |
| | peptide | VHYLDQTEQWEKFGIEKRQEALELIKKGYTQQL | |
| | from Shisa6 | AFKQPSSAYAAFNNRPPSTWLTAYVVKVFSLAA | |
| | and linker | NLIAIDSHVLCGAVKWLILEKQKPDGVFQEDGPV | |
| | | IHQEMIGGFRNAKEADVSLTAFVLIALQEARDICE | |
| | | GQVNSLPGSINKAGEYIEASYMNLQRPYTVAIAG | |
| | | YALALMNKLEEPYLGKFLNTAKDRNRWEEPDQ | |
| | | QLYNVEATSYALLALLLLKDFDSVPPVVRWLNE | |
| | | QRYYGGGYGSTQATFMVFQALAQYQTDVPDHK | |
| | | DLNMDVSFHLPSRGSGSGSARGRSGNRTLNAG | |
| | | AVGGRRAGGALARGGRELNSTARASGVPEAGS | |
| | | RRGQSAAAAAAAAAAASATVTYETCWGYYDVS | |
| | | GQYDKEFECNNSESGYLYCCGTCYYRFCCKKR | |
| | | HEKLDQRQCTNYQSPVWVQTPSTKVVSPGPEN | |
| | | KYDPEKDKTNFTVYITCGVIAFVIVAGVFAKVSYD | |
| | | KAHRPPREMNIHRALADILRQQGPIPIAHCERETI | |
| | | SAIDTSPKENTPVRSTSKNHYTPVRTAKQTPGD | |
| | | RQYNHPILSSATQTPTHEKPRMNNILTSATEPYD | |
| | | LSFSRSYQNLAHLPPSYESAVKTNPSKYSSLKR | |
| | | LTDKEADEYYMRRRHLPDLAARGTLPLNVIQMS | |
| | | QQKPLPRERPRRPIRAMSQDRVLSPRRGLPDE | |
| | | FGMPYDRILSDEQLLSTERLHSQDPLLSPERTAF | |
| | | PEQSLSRAISHTDVFVSTPVLDRYRMTKMHSHP | |
| | | SASNNSYATLGQSQTAAKRHAFASRRHNTVEQ | |
| | | LHYIPGHHTCYTASKTEVTV | |
| Excitatory | C3dg- | MALRRLLLPPLLLSLLLSLASLHLPPGADAGGV | 11 |
| | Shisa6-HA | QKVDVPAADLSDQVPDTDSETRIILQGSPVVQM | |
| | protein with | AEDAVDGERLKHLIVTPAGCGEQNMIGMTPTVIA | |
| | signal | VHYLDQTEQWEKFGIEKRQEALELIKKGYTQQL | |
| | peptide | AFKQPSSAYAAFNNRPPSTWLTAYVVKVFSLAA | |
| | from | NLIAIDSHVLCGAVKWLILEKQKPDGVFQEDGPV | |
| | Shisa6, | IHQEMIGGFRNAKEADVSLTAFVLIALQEARDICE | |
| | linker, and | GQVNSLPGSINKAGEYIEASYMNLQRPYTVAIAG | |
| | HA tag | YALALMNKLEEPYLGKFLNTAKDRNRWEEPDQ | |
| | | QLYNVEATSYALLALLLLKDFDSVPPVVRWLNE | |
| | | QRYYGGGYGSTQATFMVFQALAQYQTDVPDHK | |
| | | DLNMDVSFHLPSRGSGSGSARGRSGNRTLNAG | |
| | | AVGGRRAGGALARGGRELNSTARASGVPEAGS | |
| | | RRGQSAAAAAAAAAAASATVTYETCWGYYDVS | |
| | | GQYDKEFECNNSESGYLYCCGTCYYRFCCKKR | |
| | | HEKLDQRQCTNYQSPVWVQTPSTKVVSPGPEN | |
| | | KYDPEKDKTNFTVYITCGVIAFVIVAGVFAKVSYD | |
| | | KAHRPPREMNIHRALADILRQQGPIPIAHCERETI | |
| | | SAIDTSPKENTPVRSTSKNHYTPVRTAKQTPGD | |
| | | RQYNHPILSSATQTPTHEKPRMNNILTSATEPYD | |
| | | LSFSRSYQNLAHLPPSYESAVKTNPSKYSSLKR | |
| | | LTDKEADEYYMRRRHLPDLAARGTLPLNVIQMS | |
| | | QQKPLPRERPRRPIRAMSQDRVLSPRRGLPDE | |
| | | FGMPYDRILSDEQLLSTERLHSQDPLLSPERTAF | |
| | | PEQSLSRAISHTDVFVSTPVLDRYRMTKMHSHP | |
| | | SASNNSYATLGQSQTAAKRHAFASRRHNTVEQ | |
| | | LHYIPGHHTCYTASKTEVTVYPYDVPDYA | |
| Inhibitory | C3dg-Shisa7 | GGVQKVDVPAADLSDQVPDTDSETRIILQGSPV | 12 |
| | protein with | VQMAEDAVDGERLKHLIVTPAGCGEQNMIGMT | |
| | linker without | PTVIAVHYLDQTEQWEKFGIEKRQEALELIKKGY | |
| | signal | TQQLAFKQPSSAYAAFNNRPPSTWLTAYVVKVF | |
| | peptide or | SLAANLIAIDSHVLCGAVKWLILEKQKPDGVFQE | |
| | HA tag | DGPVIHQEMIGGFRNAKEADVSLTAFVLIALQEA | |
| | | RDICEGQVNSLPGSINKAGEYIEASYMNLQRPYT | |
| | | VAIAGYALALMNKLEEPYLGKFLNTAKDRNRWE | |
| | | EPDQQLYNVEATSYALLALLLLKDFDSVPPVVR | |
| | | WLNEQRYYGGGYGSTQATFMVFQALAQYQTD | |
| | | VPDHKDLNMDVSFHLPSRGSGSGSRPSNDTSS | |
| | | VAPGPLPALLAHLRRLTGALAGGGSAAGTSANA | |
| | | TKTSPASGTGAAARAPPPAELCHGYYDVMGQY | |
| | | DATFNCSTGSYRFCCGTCHYRFCCEHRHMRLA | |
| | | QASCSNYDTPRWATTPPPLAGGAGGAGGAGG | |
| | | GPGPGQAGWLEGGRAGGAGGRGGEGPGGST | |
| | | AYVVCGVISFALAVGVGAKVAFSKASRAPRAHR | |
| | | EINVPRALVDILRHQAGPATRPDRARSSSLTPGL | |
| | | GGPDSMAPRTPKNLYNTMKPSNLDNLHYNVNS | |
| | | PKHHAATLDWRAMPPPSPSLHYSTLSCSRSFH | |
| | | NLSHLPPSYEAAVKSELNRYSSLKRLAEKDLDE | |
| | | AYLKRRQLEMPRGTLPLHALRRPGTGGGYRMD | |
| | | GWGGPEELGLAPAPNPRRVMSQEHLLGDGSR | |
| | | ASRYEFTLPRARLVSQEHLLLSSPEALRQSREH | |
| | | LLSPPRSPALPPDPTTRASLAASHSNLLLGPGG | |
| | | PPTPLHGLPPSGLHAHHHHALHGSPQPAWMSD | |
| | | AGGGGGTLARRPPFQRQGTLEQLQFIPGHHLP | |
| | | QHLRTASKNEVTV | |
| Inhibitory | C3dg-Shisa7 | MPALLLLGTVALLASAAGPAGAGGVQKVDVPA | 13 |
| | protein with | ADLSDQVPDTDSETRIILQGSPVVQMAEDAVDG | |
| | signal | ERLKHLIVTPAGCGEQNMIGMTPTVIAVHYLDQT | |
| | peptide | EQWEKFGIEKRQEALELIKKGYTQQLAFKQPSS | |
| | from Shisa7 | AYAAFNNRPPSTWLTAYVVKVFSLAANLIAIDSH | |
| | and linker | VLCGAVKWLILEKQKPDGVFQEDGPVIHQEMIG | |
| | | GFRNAKEADVSLTAFVLIALQEARDICEGQVNSL | |
| | | PGSINKAGEYIEASYMNLQRPYTVAIAGYALALM | |
| | | NKLEEPYLGKFLNTAKDRNRWEEPDQQLYNVE | |
| | | ATSYALLALLLLKDFDSVPPVVRWLNEQRYYGG | |
| | | GYGSTQATFMVFQALAQYQTDVPDHKDLNMDV | |
| | | SFHLPSRGSGSGSRPSNDTSSVAPGPLPALLAH | |
| | | LRRLTGALAGGGSAAGTSANATKTSPASGTGAA | |
| | | ARAPPPAELCHGYYDVMGQYDATFNCSTGSYR | |
| | | FCCGTCHYRFCCEHRHMRLAQASCSNYDTPR | |
| | | WATTPPPLAGGAGGAGGAGGGPGPGQAGWLE | |
| | | GGRAGGAGGRGGEGPGGSTAYVVCGVISFALA | |
| | | VGVGAKVAFSKASRAPRAHREINVPRALVDILRH | |
| | | QAGPATRPDRARSSSLTPGLGGPDSMAPRTPK | |
| | | NLYNTMKPSNLDNLHYNVNSPKHHAATLDWRA | |
| | | MPPPSPSLHYSTLSCSRSFHNLSHLPPSYEAAV | |
| | | KSELNRYSSLKRLAEKDLDEAYLKRRQLEMPRG | |
| | | TLPLHALRRPGTGGGYRMDGWGGPEELGLAPA | |
| | | PNPRRVMSQEHLLGDGSRASRYEFTLPRARLV | |
| | | SQEHLLLSSPEALRQSREHLLSPPRSPALPPDP | |
| | | TTRASLAASHSNLLLGPGGPPTPLHGLPPSGLH | |
| | | AHHHHALHGSPQPAWMSDAGGGGGTLARRPP | |
| | | FQRQGTLEQLQFIPGHHLPQHLRTASKNEVTV | |
| Inhibitory | C3dg- | MPALLLLGTVALLASAAGPAGAGGVQKVDVPA | 14 |
| | Shisa7-HA | ADLSDQVPDTDSETRIILQGSPVVQMAEDAVDG | |
| | protein with | ERLKHLIVTPAGCGEQNMIGMTPTVIAVHYLDQT | |
| | signal | EQWEKFGIEKRQEALELIKKGYTQQLAFKQPSS | |
| | peptide | AYAAFNNRPPSTWLTAYVVKVFSLAANLIAIDSH | |
| | from Shisa7 | VLCGAVKWLILEKQKPDGVFQEDGPVIHQEMIG | |
| | linker, | GFRNAKEADVSLTAFVLIALQEARDICEGQVNSL | |
| | and | PGSINKAGEYIEASYMNLQRPYTVAIAGYALALM | |
| | HA tag | NKLEEPYLGKFLNTAKDRNRWEEPDQQLYNVE | |
| | | ATSYALLALLLLKDFDSVPPVVRWLNEQRYYGG | |
| | | GYGSTQATFMVFQALAQYQTDVPDHKDLNMDV | |
| | | SFHLPSRGSGSGSRPSNDTSSVAPGPLPALLAH | |
| | | LRRLTGALAGGGSAAGTSANATKTSPASGTGAA | |
| | | ARAPPPAELCHGYYDVMGQYDATFNCSTGSYR | |
| | | FCCGTCHYRFCCEHRHMRLAQASCSNYDTPR | |
| | | WATTPPPLAGGAGGAGGAGGGPGPGQAGWLE | |
| | | GGRAGGAGGRGGEGPGGSTAYVVCGVISFALA | |
| | | VGVGAKVAFSKASRAPRAHREINVPRALVDILRH | |
| | | QAGPATRPDRARSSSLTPGLGGPDSMAPRTPK | |
| | | NLYNTMKPSNLDNLHYNVNSPKHHAATLDWRA | |
| | | MPPPSPSLHYSTLSCSRSFHNLSHLPPSYEAAV | |
| | | KSELNRYSSLKRLAEKDLDEAYLKRRQLEMPRG | |
| | | TLPLHALRRPGTGGGYRMDGWGGPEELGLAPA | |
| | | PNPRRVMSQEHLLGDGSRASRYEFTLPRARLV | |
| | | SQEHLLLSSPEALRQSREHLLSPPRSPALPPDP | |
| | | TTRASLAASHSNLLLGPGGPPTPLHGLPPSGLH | |
| | | AHHHHALHGSPQPAWMSDAGGGGGTLARRPP | |
| | | FQRQGTLEQLQFIPGHHLPQHLRTASKNEVTVY | |
| | | PYDVPDYA | |
| Excitatory | Gas6-Shisa6 | FSGTPVIRLRFKRLQPTRLLAEFDFRTFDPEGVL | 15 |
| | protein with | FFAGGRSDSTWIVLGLRAGRLELQLRYNGVGRI | |
| | linker without | TSSGPTINHGMWQTISVEELERNLVIKVNKDAV | |
| | signal | MKIAVAGELFQLERGLYHLNLTVGGIPFKESELV | |
| | peptide and | QPINPRLDGCMRSWNWLNGEDSAIQETVKANT | |
| | HA tag | KMQCFSVTERGSFFPGNGFATYRLNYTRTSLDV | |
| | | GTETTWEVKVVARIRPATDTGVLLALVGDDDVV | |
| | | PISVALVDYHSTKKLKKQLVVLAVEDVALALMEIK | |
| | | VCDSQEHTVTVSLREGEATLEVDGTKGQSEVST | |
| | | AQLQERLDTLKTHLQGSVHTYVGGLPEVSVISA | |
| | | PVTAFYRGCMTLEVNGKILDLDTASYKHSDITSH | |
| | | SCPPVEHATPGSGSGSARGRSGNRTLNAGAVG | |
| | | GRRAGGALARGGRELNSTARASGVPEAGSRR | |
| | | GQSAAAAAAAAAAASATVTYETCWGYYDVSGQ | |
| | | YDKEFECNNSESGYLYCCGTCYYRFCCKKRHE | |
| | | KLDQRQCTNYQSPVWVQTPSTKVVSPGPENKY | |
| | | DPEKDKTNFTVYITCGVIAFVIVAGVFAKVSYDKA | |
| | | HRPPREMNIHRALADILRQQGPIPIAHCERETISA | |
| | | IDTSPKENTPVRSTSKNHYTPVRTAKQTPGDRQ | |
| | | YNHPILSSATQTPTHEKPRMNNILTSATEPYDLS | |
| | | FSRSYQNLAHLPPSYESAVKTNPSKYSSLKRLT | |
| | | DKEADEYYMRRRHLPDLAARGTLPLNVIQMSQ | |
| | | QKPLPRERPRRPIRAMSQDRVLSPRRGLPDEF | |
| | | GMPYDRILSDEQLLSTERLHSQDPLLSPERTAFP | |
| | | EQSLSRAISHTDVFVSTPVLDRYRMTKMHSHPS | |
| | | ASNNSYATLGQSQTAAKRHAFASRRHNTVEQL | |
| | | HYIPGHHTCYTASKTEVTV | |
| Excitatory | Gas6-Shisa6 | MALRRLLLPPLLLSLLLSLASLHLPPGADAFSG | 16 |
| | protein with | TPVIRLRFKRLQPTRLLAEFDFRTFDPEGVLFFA | |
| | signal | GGRSDSTWIVLGLRAGRLELQLRYNGVGRITSS | |
| | peptide | GPTINHGMWQTISVEELERNLVIKVNKDAVMKIA | |
| | from Shisa6 | VAGELFQLERGLYHLNLTVGGIPFKESELVQPIN | |
| | and linker | PRLDGCMRSWNWLNGEDSAIQETVKANTKMQ | |
| | | CFSVTERGSFFPGNGFATYRLNYTRTSLDVGTE | |
| | | TTWEVKVVARIRPATDTGVLLALVGDDDVVPISV | |
| | | ALVDYHSTKKLKKQLVVLAVEDVALALMEIKVCD | |
| | | SQEHTVTVSLREGEATLEVDGTKGQSEVSTAQL | |
| | | QERLDTLKTHLQGSVHTYVGGLPEVSVISAPVT | |
| | | AFYRGCMTLEVNGKILDLDTASYKHSDITSHSCP | |
| | | PVEHATPGSGSGSARGRSGNRTLNAGAVGGR | |
| | | RAGGALARGGRELNSTARASGVPEAGSRRGQS | |
| | | AAAAAAAAAAASATVTYETCWGYYDVSGQYDK | |
| | | EFECNNSESGYLYCCGTCYYRFCCKKRHEKLD | |
| | | QRQCTNYQSPVWVQTPSTKVVSPGPENKYDPE | |
| | | KDKTNFTVYITCGVIAFVIVAGVFAKVSYDKAHR | |
| | | PPREMNIHRALADILRQQGPIPIAHCERETISAID | |
| | | TSPKENTPVRSTSKNHYTPVRTAKQTPGDRQY | |
| | | NHPILSSATQTPTHEKPRMNNILTSATEPYDLSF | |
| | | SRSYQNLAHLPPSYESAVKTNPSKYSSLKRLTD | |
| | | KEADEYYMRRRHLPDLAARGTLPLNVIQMSQQK | |
| | | PLPRERPRRPIRAMSQDRVLSPRRGLPDEFGM | |
| | | PYDRILSDEQLLSTERLHSQDPLLSPERTAFPEQ | |
| | | SLSRAISHTDVFVSTPVLDRYRMTKMHSHPSAS | |
| | | NNSYATLGQSQTAAKRHAFASRRHNTVEQLHYI | |
| | | PGHHTCYTASKTEVTV | |
| Excitatory | Gas6-Shisa6 | MALRRLLLPPLLLSLLLSLASLHLPPGADAFSG | 17 |
| | Protein with | TPVIRLRFKRLQPTRLLAEFDFRTFDPEGVLFFA | |
| | signal | GGRSDSTWIVLGLRAGRLELQLRYNGVGRITSS | |
| | peptide | GPTINHGMWQTISVEELERNLVIKVNKDAVMKIA | |
| | from Shisa6 | VAGELFQLERGLYHLNLTVGGIPFKESELVQPIN | |
| | and HA tag | PRLDGCMRSWNWLNGEDSAIQETVKANTKMQ | |
| | | CFSVTERGSFFPGNGFATYRLNYTRTSLDVGTE | |
| | | TTWEVKVVARIRPATDTGVLLALVGDDDVVPISV | |
| | | ALVDYHSTKKLKKQLVVLAVEDVALALMEIKVCD | |
| | | SQEHTVTVSLREGEATLEVDGTKGQSEVSTAQL | |
| | | QERLDTLKTHLQGSVHTYVGGLPEVSVISAPVT | |
| | | AFYRGCMTLEVNGKILDLDTASYKHSDITSHSCP | |
| | | PVEHATPGSGSGSARGRSGNRTLNAGAVGGR | |
| | | RAGGALARGGRELNSTARASGVPEAGSRRGQS | |
| | | AAAAAAAAAAASATVTYETCWGYYDVSGQYDK | |
| | | EFECNNSESGYLYCCGTCYYRFCCKKRHEKLD | |
| | | QRQCTNYQSPVWVQTPSTKVVSPGPENKYDPE | |
| | | KDKTNFTVYITCGVIAFVIVAGVFAKVSYDKAHR | |
| | | PPREMNIHRALADILRQQGPIPIAHCERETISAID | |
| | | TSPKENTPVRSTSKNHYTPVRTAKQTPGDRQY | |
| | | NHPILSSATQTPTHEKPRMNNILTSATEPYDLSF | |
| | | SRSYQNLAHLPPSYESAVKTNPSKYSSLKRLTD | |
| | | KEADEYYMRRRHLPDLAARGTLPLNVIQMSQQK | |
| | | PLPRERPRRPIRAMSQDRVLSPRRGLPDEFGM | |
| | | PYDRILSDEQLLSTERLHSQDPLLSPERTAFPEQ | |
| | | SLSRAISHTDVFVSTPVLDRYRMTKMHSHPSAS | |
| | | NNSYATLGQSQTAAKRHAFASRRHNTVEQLHYI | |
| | | PGHHTCYTASKTEVTVYPYDVPDYA | |
| Inhibitory | Gas6-Shisa7 | FSGTPVIRLRFKRLQPTRLLAEFDFRTFDPEGVL | 18 |
| | protein with | FFAGGRSDSTWIVLGLRAGRLELQLRYNGVGRI | |
| | linker without | TSSGPTINHGMWQTISVEELERNLVIKVNKDAV | |
| | signal | MKIAVAGELFQLERGLYHLNLTVGGIPFKESELV | |
| | peptide and | QPINPRLDGCMRSWNWLNGEDSAIQETVKANT | |
| | HA tag. | KMQCFSVTERGSFFPGNGFATYRLNYTRTSLDV | |
| | | GTETTWEVKVVARIRPATDTGVLLALVGDDDVV | |
| | | PISVALVDYHSTKKLKKQLVVLAVEDVALALMEIK | |
| | | VCDSQEHTVTVSLREGEATLEVDGTKGQSEVST | |
| | | AQLQERLDTLKTHLQGSVHTYVGGLPEVSVISA | |
| | | PVTAFYRGCMTLEVNGKILDLDTASYKHSDITSH | |
| | | SCPPVEHATPGSGSGSRPSNDTSSVAPGPLPA | |
| | | LLAHLRRLTGALAGGGSAAGTSANATKTSPASG | |
| | | TGAAARAPPPAELCHGYYDVMGQYDATFNCST | |
| | | GSYRFCCGTCHYRFCCEHRHMRLAQASCSNY | |
| | | DTPRWATTPPPLAGGAGGAGGAGGGPGPGQA | |
| | | GWLEGGRAGGAGGRGGEGPGGSTAYVVCGVI | |
| | | SFALAVGVGAKVAFSKASRAPRAHREINVPRAL | |
| | | VDILRHQAGPATRPDRARSSSLTPGLGGPDSMA | |
| | | PRTPKNLYNTMKPSNLDNLHYNVNSPKHHAATL | |
| | | DWRAMPPPSPSLHYSTLSCSRSFHNLSHLPPSY | |
| | | EAAVKSELNRYSSLKRLAEKDLDEAYLKRRQLE | |
| | | MPRGTLPLHALRRPGTGGGYRMDGWGGPEEL | |
| | | GLAPAPNPRRVMSQEHLLGDGSRASRYEFTLP | |
| | | RARLVSQEHLLLSSPEALRQSREHLLSPPRSPA | |
| | | LPPDPTTRASLAASHSNLLLGPGGPPTPLHGLP | |
| | | PSGLHAHHHHALHGSPQPAWMSDAGGGGGTL | |
| | | ARRPPFQRQGTLEQLQFIPGHHLPQHLRTASKN | |
| | | EVTV | |
| Inhibitory | Gas6-Shisa7 | MPALLLLGTVALLASAAGPAGAFSGTPVIRLRF | 19 |
| | Protein w | KRLQPTRLLAEFDFRTFDPEGVLFFAGGRSDST | |
| | signal | WIVLGLRAGRLELQLRYNGVGRITSSGPTINHG | |
| | peptide | MWQTISVEELERNLVIKVNKDAVMKIAVAGELFQ | |
| | from Shisa7 | LERGLYHLNLTVGGIPFKESELVQPINPRLDGCM | |
| | and linker | RSWNWLNGEDSAIQETVKANTKMQCFSVTERG | |
| | | SFFPGNGFATYRLNYTRTSLDVGTETTWEVKVV | |
| | | ARIRPATDTGVLLALVGDDDVVPISVALVDYHST | |
| | | KKLKKQLVVLAVEDVALALMEIKVCDSQEHTVTV | |
| | | SLREGEATLEVDGTKGQSEVSTAQLQERLDTLK | |
| | | THLQGSVHTYVGGLPEVSVISAPVTAFYRGCMT | |
| | | LEVNGKILDLDTASYKHSDITSHSCPPVEHATPG | |
| | | SGSGSRPSNDTSSVAPGPLPALLAHLRRLTGAL | |
| | | AGGGSAAGTSANATKTSPASGTGAAARAPPPA | |
| | | ELCHGYYDVMGQYDATFNCSTGSYRFCCGTCH | |
| | | YRFCCEHRHMRLAQASCSNYDTPRWATTPPPL | |
| | | AGGAGGAGGAGGGPGPGQAGWLEGGRAGGA | |
| | | GGRGGEGPGGSTAYVVCGVISFALAVGVGAKV | |
| | | AFSKASRAPRAHREINVPRALVDILRHQAGPATR | |
| | | PDRARSSSLTPGLGGPDSMAPRTPKNLYNTMK | |
| | | PSNLDNLHYNVNSPKHHAATLDWRAMPPPSPS | |
| | | LHYSTLSCSRSFHNLSHLPPSYEAAVKSELNRY | |
| | | SSLKRLAEKDLDEAYLKRRQLEMPRGTLPLHAL | |
| | | RRPGTGGGYRMDGWGGPEELGLAPAPNPRRV | |
| | | MSQEHLLGDGSRASRYEFTLPRARLVSQEHLLL | |
| | | SSPEALRQSREHLLSPPRSPALPPDPTTRASLA | |
| | | ASHSNLLLGPGGPPTPLHGLPPSGLHAHHHHAL | |
| | | HGSPQPAWMSDAGGGGGTLARRPPFQRQGTL | |
| | | EQLQFIPGHHLPQHLRTASKNEVTV | |
| Inhibitory | Gas6-Shisa7 | MPALLLLGTVALLASAAGPAGAFSGTPVIRLRF | 20 |
| | Protein w | KRLQPTRLLAEFDFRTFDPEGVLFFAGGRSDST | |
| | signal | WIVLGLRAGRLELQLRYNGVGRITSSGPTINHG | |
| | peptide | MWQTISVEELERNLVIKVNKDAVMKIAVAGELFQ | |
| | from Shisa7, | LERGLYHLNLTVGGIPFKESELVQPINPRLDGCM | |
| | linker | RSWNWLNGEDSAIQETVKANTKMQCFSVTERG | |
| | and | SFFPGNGFATYRLNYTRTSLDVGTETTWEVKVV | |
| | HA tag | ARIRPATDTGVLLALVGDDDVVPISVALVDYHST | |
| | | KKLKKQLVVLAVEDVALALMEIKVCDSQEHTVTV | |
| | | SLREGEATLEVDGTKGQSEVSTAQLQERLDTLK | |
| | | THLQGSVHTYVGGLPEVSVISAPVTAFYRGCMT | |
| | | LEVNGKILDLDTASYKHSDITSHSCPPVEHATPG | |
| | | SGSGSRPSNDTSSVAPGPLPALLAHLRRLTGAL | |
| | | AGGGSAAGTSANATKTSPASGTGAAARAPPPA | |
| | | ELCHGYYDVMGQYDATFNCSTGSYRFCCGTCH | |
| | | YRFCCEHRHMRLAQASCSNYDTPRWATTPPPL | |
| | | AGGAGGAGGAGGGPGPGQAGWLEGGRAGGA | |
| | | GGRGGEGPGGSTAYVVCGVISFALAVGVGAKV | |
| | | AFSKASRAPRAHREINVPRALVDILRHQAGPATR | |
| | | PDRARSSSLTPGLGGPDSMAPRTPKNLYNTMK | |
| | | PSNLDNLHYNVNSPKHHAATLDWRAMPPPSPS | |
| | | LHYSTLSCSRSFHNLSHLPPSYEAAVKSELNRY | |
| | | SSLKRLAEKDLDEAYLKRRQLEMPRGTLPLHAL | |
| | | RRPGTGGGYRMDGWGGPEELGLAPAPNPRRV | |
| | | MSQEHLLGDGSRASRYEFTLPRARLVSQEHLLL | |
| | | SSPEALRQSREHLLSPPRSPALPPDPTTRASLA | |
| | | ASHSNLLLGPGGPPTPLHGLPPSGLHAHHHHAL | |
| | | HGSPQPAWMSDAGGGGGTLARRPPFQRQGTL | |
| | | EQLQFIPGHHLPQHLRTASKNEVTVYPYDVPDY | |
| | | A | |

### (b) Fusion Protein System

Further aspects of the present disclosure are directed to a set of fusion proteins which act together in a system to label both a presynaptic and postsynaptic terminal of a target synapse at the same time. Accordingly, this aspect of the disclosure is referred to herein as a “fusion protein system”.

In various aspects, the fusion protein system comprises two fusion proteins. In various aspects, the first fusion protein can comprise an N terminal domain comprising a first fragment of a activated glial receptor binding domain and a C terminal domain comprising a transmembrane domain of a presynaptic protein; and the second fusion protein can comprise an N terminal domain comprising a second fragment of an activated glial receptor binding domain and a C terminal domain comprising transmembrane domain of a postsynaptic protein. In various aspects, the first fragment and the second fragment of the activated glial receptor binding domain are complementary and capable of complexing or associating to form a fully active “activated glial receptor binding domain”.

In various aspects, the activated glial receptor binding domain used to derive the first and second fragments is as described above for the single fusion protein. For example, the activated glial receptor binding domain may be optionally derived from a complement protein or a Gas6 peptide as described above.

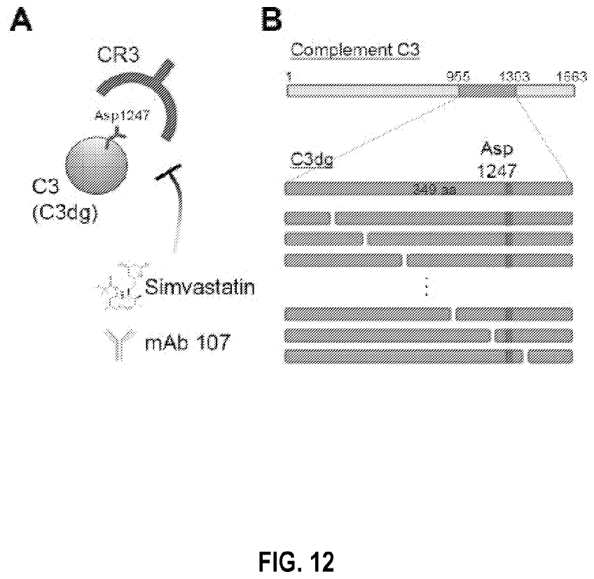

In some aspects, the first and second fragments used in the first and second fusion proteins herein may be derived from a complement protein. For example, the first and second fragment may be derived from a Complement C3 protein or activated glial receptor binding domain thereof. In some aspects, the first and second fragments may be derived from a C3dg peptide. The binding motif of C3 (C3dg) contains a Aspartate (Asp) residue for CR3 binding. Accordingly, in various aspects, when first and second fragments of the C3dg peptide are prepared for inclusion in the first and second fusion proteins herein, the C3dg peptide may be divided so that the aspartate residue for CR3 binding is included in one of the two fragments. In various aspects, the C3dg peptide may be divided as shown in FIG. 12B, where the aspartate residue is labeled in red.

Likewise, the first and second fragments included in the first and second fusion proteins herein may be derived from a Gas6 protein or peptide. As described above, an exemplary region of the Gas6 protein that interacts with a receptor expressed by activated glia is the LG1/LG2 domain. Accordingly, in some aspects, the first and second fragments included in the first and second fusion proteins herein may be derived from an LG1/LG2 domain of a Gas 6 protein. As with the C3dg peptide above, in silico sequence analysis can be used to analyze the Gas6 protein at the LG1/LG2 domain and predict suitable fragments.

In various aspects, as described, the first and second fusion proteins of the fusion protein set comprise, respectively, a transmembrane domain of a presynaptic protein (first fusion protein) or a postsynaptic protein (second fusion protein). Exemplary post-synaptic proteins are described above and can comprise, for example, Shisa6 or Shisa7. Exemplary presynaptic proteins that can be used include synaptophysin.

In further aspects, the first and second fusion proteins may each further comprise complementary fragments of another protein—such as a fluorophore. In these aspects, the first fusion protein (i.e., the protein localized to the presynaptic terminal when expressed) comprises a first fragment of a fluorophore and the second fusion protein (i.e, the protein localized to the postsynaptic terminal when expressed) comprises a second fragment of the same fluorophore such that the first fragment and the second fragment of the fluorophore can combine, complex or otherwise associate to generate a signal. The signal may be an optical signal. Suitable fluorophores that may be fragmented for use in the first and second fusion proteins include, for example CFP (Cerulean Fluorescent Protein), YFP (Yellow Fluorescent Protein also known as Venus), RFP (red fluorescent protein) or mCherry variants.

The first and second fusion proteins may also optionally comprise a signal peptide to mediate expression on the surface of the target neuron. Accordingly, the signal peptide may be at the N terminus or may optionally be included in the transmembrane fragment (i.e., be located at the N-terminus of the transmembrane domain. In some aspects, the signal peptide may be derived from the presynaptic or postsynaptic protein. When derived from postsynaptic protein, the signal peptide can comprise SEQ ID NO: 6 for Shisa6 or 7 for Shisa7. In some aspects, the signal peptide may be derived from a mouse immunoglobulin kappa.

In view of the foregoing, a first fusion proteins of the fusion protein system, then, may comprise: N-terminus—(a1) an optional signal peptide (e.g, a signal peptide derived from the presynaptic protein), (b1) an optional first fragment of a fluorophore, (c1) a first fragment of the activated glial receptor binding domain, (d1) an optional peptide linker, (e1) an optional second signal peptide (e.g, a signal peptide derived from mouse immunoglobulin kappa), and (f1) transmembrane domain of a presynaptic protein—C terminus. The corresponding fusion protein in the system (i.e., the second fusion protein) may comprise: N-terminus—(a2) an optional signal peptide (e.g., a signal peptide derived from the postsynaptic protein), (b2) a second fragment of a fluorophore (if b1 is included above), (c2) a second fragment of the activated glial receptor binding domain (complementary to and able to complex with c1 to form a functional activated glial receptor binding domain), (d2) an optional peptide linker, (e2) an optional second signal peptide (e.g, a signal peptide derived from mouse immunoglobulin kappa), and (f2) transmembrane domain of a postsynaptic protein—C terminus.

### (c) Nucleic Acids/Expression Vectors

In accordance with various aspects of the present disclosure, the fusion proteins described herein can be provided in one or more nucleic acids encoding by the fusion proteins or set of fusion proteins as described above.

As used herein, the term “nucleic acid sequence,” “nucleic acid molecule,” “polynucleotide,” and “oligonucleotide” are used interchangeably and refer to a polymeric form of nucleotides that may have various lengths, either deoxyribonucleotides or ribonucleotides, or analogs thereof. Nucleic acid molecules can be deoxyribonucleic acid (DNA) or ribonucleic acid (RNA) fragments generated, for example, by a polymerase chain reaction (PCR) or by in vitro translation, and fragments generated by any one or more of ligation, scission, endonuclease action, or exonuclease action. Nucleic acid molecules can be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination thereof. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, morpholino, or the like. Nucleic acid molecules can be either single stranded or double stranded (e.g., ssDNA, dsDNA, ssRNA, or dsRNA).

The term “nucleotide” refers to sequences with conventional nucleotide bases, sugar residues and internucleotide phosphate linkages, but also to those that contain modifications of any or all of these moieties. The term “nucleotide” as used herein includes those moieties that contain not only the natively found purine and pyrimidine bases adenine (A), guanine (G), thymine (T), cytosine (C), and uracil (U), but also modified or analogous forms thereof. Polynucleotides include RNA and DNA sequences of more than one nucleotide in a single chain. Modified RNA or modified DNA, as used herein, refers to a nucleic acid molecule in which one or more of the components of the nucleic acid, namely sugars, bases, and phosphate moieties, are different from that which occurs in nature.

As used herein, the term “isolated” nucleic acid molecule (e.g., an isolated DNA, isolated cDNA, or an isolated vector genome) means a nucleic acid molecule separated or substantially free from at least some of the other components of the naturally occurring organism or virus, for example, the cell or viral structural components or other polypeptides or nucleic acids commonly found associated with the nucleic acid.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

Accordingly, in certain aspects of the disclosure, an isolated nucleic acid is provided a nucleic acid encoding for a single fusion protein as described herein (e.g., Section (a)). Also provided are sets of nucleic acids (e.g., a pair of nucleic acids) where each encoding one fusion protein of the fusion protein set provided herein (e.g., Section (b)). That is, the provided sets of nucleic acids comprise in various aspects a first nucleic acid encoding a first fusion protein (i.e., comprising an N terminal domain comprising a first fragment of a activated glial receptor binding domain and a C terminal domain comprising a transmembrane domain of a presynaptic protein) and a second nucleic acid encoding a second fusion protein (i.e., comprising an N terminal domain comprising a second fragment of a activated glial receptor binding domain and a C terminal domain comprising a transmembrane domain of a postsynaptic protein).

As mentioned above, the C3dg peptides and Gas6 peptides that may be used in the fusion proteins herein are fragments of full length C3 and Gas6 proteins, respectively. The full length mRNA for the C3 protein is provided herein as SEQ ID NO: 37 where nucleotides 995-1303 encode for C3dg specifically (e.g., SEQ ID NO: 21). The full length mRNA for the Gas6 protein is provided herein as SEQ ID NO: 38, where nucleotides 1088-2218 encode the Gas6 peptide herein (e.g., L1/L2 domain, SEQ ID NO: 22). SEQ ID NOs 21 and 22 are provided in Table 2 below along with additional exemplary nucleic acids (SEQ ID NOs 23-32) for encoding one or more components of the fusion proteins described herein (e.g., Shisa6 or Shisa7 proteins, signal peptides, linkers, and/or HA tags). Where appropriate, the nucleic acids are annotated to indicate coding regions for one or more of the components listed above.

TABLE
Illustrative Nucleic Acids

| Nucleic Acid | | SEQ ID |
|---|---|---|
| Description | Sequence | NO: |
| C3dg coding sequence | gggggagtgcagaaggtggatgtgcctgccgcagaccttagcgaccaa | 21 |
| | gtgccagacacagactctgagaccagaattatcctgcaagggagcccg | |
| | gtggttcagatggctgaagatgctgtggacggggagcggctgaaacacc | |
| | tgatcgtgacccccgcaggctgtggggaacagaacatgattggcatgac | |
| | accaacagtcattgcggtacactacctggaccagaccgaacagtggga | |
| | gaagttcggcatagagaagaggcaagaggccctggagctcatcaaga | |
| | aagggtacacccagcagctggccttcaaacagcccagctctgcctatgct | |
| | gccttcaacaaccggccccccagcacctggctgacagcctacgtggtca | |
| | aggtcttctctctagctgccaacctcatcgccatcgactctcacgtcctgtgt | |
| | ggggctgttaaatggttgattctggagaaacagaagccggatggtgtctttc | |
| | aggaggatgggcccgtgattcaccaagaaatgattggtggcttccggaa | |
| | cgccaaggaggcagatgtgtcactcacagccttcgtcctcatcgcactgc | |
| | aggaagccagggacatctgtgaggggcaggtcaatagccttcctggga | |
| | gcatcaacaaggcaggggagtatattgaagccagttacatgaacctgca | |
| | gagaccatacacagtggccattgctgggtatgccctggccctgatgaaca | |
| | aactggaggaaccttacctcggcaagtttctgaacacagccaaagatcg | |
| | gaaccgctgggaggagcctgaccagcagctctacaacgtagaggcca | |
| | catcctacgccctcctggccctgctgctgctgaaagactttgactctgtgcc | |
| | ccctgtagtgcgctggctcaatgagcaaagatactacggaggcggctatg | |
| | gctccacccaggctaccttcatggtattccaagccttggcccaatatcaaa | |
| | cagatgtccctgaccataaggacttgaacatggatgtgtccttccacctccc | |
| | cagccgt | |
| Gas6 L1/L2 coding | ttcagcgggacccccgtgattagactacgcttcaagaggcttcagc | 22 |
| sequence | ctaccaggctgctggctgaatttgacttccgcacttttgaccctgaag | |
| | gagtcctcttcttcgctggaggccgttcagacagcacctggattgtcc | |
| | tgggcctaagagctgggcggcttgagctgcagcttcggtacaatgg | |
| | cgttgggcgcatcaccagcagcgggccaaccatcaaccacggc | |
| | atgtggcaaactatctccgtggaagagctggaacgtaaccttgtcat | |
| | caaggtcaacaaagatgctgtaatgaagatcgcggtagctgggg | |
| | agctgtttcagctggagaggggcctctatcacctgaatctcaccgtg | |
| | ggcggcattcccttcaaggagagtgagctcgtccagccgattaacc | |
| | ctcgcctggatgggtgcatgaggagttggaactggctgaacgggg | |
| | aagacagcgccatccaggagacagtcaaggcaaacacaaaaa | |
| | tgcagtgcttctctgtgacagaaaggggctccttcttcccggggaat | |
| | ggatttgctacctacaggctcaactacacccgaacatcgctggatgt | |
| | cggcacggaaaccacctgggaagttaaagttgtggctcggatccg | |
| | ccctgccacggacacgggggtgctgctggcgctggtgggggacg | |
| | acgatgtcgtccccatctctgtggccctagtcgactaccactctacaa | |
| | agaagctcaagaagcagttggtggtcctggcagttgaggatgttgc | |
| | cctggcactgatggaaatcaaggtgtgcgacagccaggaacaca | |
| | cggtcactgtctccctgcgggagggtgaggccaccctagaagtgg | |
| | atggcacaaagggccagagtgaagtgagcactgcccagctgca | |
| | ggagcgactggacacacttaagacacatctgcaaggctctgtgca | |
| | cacctatgttggaggcctgccagaagtatcggtgatttctgcacccgt | |
| | cactgcgttctaccgcggatgcatgactctggaggtaaacgggaa | |
| | aatcctggacctggatacggcctcgtacaagcacagtgacatcac | |
| | ctcccactcctgcccgcctgtggagcatgccaccccc | |
| Shisa6 coding sequence | gcccgcggccgctccgggaaccggaccctgaacgcgggagccgtcgg | 23 |
| | aggtcggcgtgctgggggcgccctggcccgaggcggccgcgaactaa | |
| | acagcaccgcccgagcgtccggcgtcccggaggcgggcagccggcg | |
| | gggacagtccgcagcagcggcagcggcggcggcggcggcggccag | |
| | cgcgactgttacttacgagacgtgctggggctactacgacgtgagcggcc | |
| | agtacgacaaggagttcgagtgcaacaacagcgagagcggctacctgt | |
| | actgctgcggcacctgctactatcgcttctgctgcaagaagcgccacgag | |
| | aagctggaccagcgccagtgcaccaactaccaaagccccgtatgggtg | |
| | cagacgcccagcaccaaggtagtgtcgccggggcccgagaacaagta | |
| | cgacccggagaaggacaagaccaacttcaccgtctacatcacttgcgg | |
| | ggtgatcgccttcgtcatcgtggcgggcgtcttcgccaaggtctcctatgac | |
| | aaggcccaccgccctccgcgagagatgaacatccacagggctctggct | |
| | gacattctaagacaacagggaccaatccccatagcacactgtgaaaga | |
| | gaaaccatctcggccatcgatacctctcccaaagagaacacgccggtcc | |
| | gatcaacctccaaaaaccactacacccctgtgcgcacagccaagcaga | |
| | ctccaggtgatcgtcagtataatcatccgatcttaagcagcgctacccaga | |
| | cccctacacatgagaagccacggatgaataacattctgacgtcggccac | |
| | ggaaccctatgacctctccttctcacgctcttaccagaacttagcccacttg | |
| | cctccatcatatgaatctgcagtgaaaaccaatccaagcaagtactcgtct | |
| | ctgaagaggctaacggacaaggaagctgatgagtattacatgagaagg | |
| | aggcacctgccagaccttgcagcccgtggtaccctccccctcaatgtcatc | |
| | cagatgtctcaacagaagccacttcctcgagaacggccacgcaggccc | |
| | atcagggccatgtcccaggacagggtcttgtctccacgtcggggattgcc | |
| | agatgaattcggcatgccctatgaccgcatcttgtctgatgaacagctgctc | |
| | tccacagagcgcctgcactcccaggacccgttgctgtccccagagagga | |
| | cagccttcccggagcagtcgctgtcgcgggccatctcgcatacggacgtc | |
| | tttgtgtccacgccagtgctggaccgctaccgcatgaccaagatgcactcc | |
| | catcccagtgcctccaataactcctatgccaccctgggccagagccaga | |
| | cagcagccaagcgccacgcctttgcctctcgcagacacaacacggtgg | |
| | agcagttacactatatcccaggccatcacacctgctacacagccagcaa | |
| | gactgaagtgaccgtg | |
| Shisa7 coding sequence | cgcccatccaacgacacaagctcagtggccccgggcccgctgcccgcg | 24 |
| | ctactcgcgcacctgcggcgcttgaccggggctctggcgggcggcggga | |
| | gcgcggcaggtaccagcgccaacgccaccaagaccagccccgcgag | |
| | tggcacgggcgcagcggcacgggcgcctcctccggccgagctctgcca | |
| | tggctactacgatgtcatgggccagtacgacgccaccttcaactgcagca | |
| | ccggctcctaccgcttctgttgtggcacctgccactaccgtttctgctgcgag | |
| | caccgccacatgcgcctggcgcaggcctcctgctccaactacgacacgc | |
| | cacgctgggccaccacgcccccgccgctggctggaggcgccgggggc | |
| | gctgggggtgcgggtgggggaccagggccgggccaggcagggtggct | |
| | ggaagggggccgggccgggggcgctgggggacgtgggggagaggg | |
| | cccagggggcagcacagcctacgtggtgtgcggagtcatcagtttcgcc | |
| | ctggcggtgggcgtcggtgccaaagtggccttcagcaaggcgtcacgtg | |
| | cacccagggcgcaccgggagatcaacgtgcccagagctctcgtggata | |
| | ttctcaggcatcaagcaggacctgcaacccgcccggaccgggccagaa | |
| | gcagttctctgaccccagggctgggaggcccagacagcatggccccaa | |
| | ggacacccaagaacctttacaacaccatgaagccctccaacctcgataa | |
| | cctgcactacaacgtcaacagccccaagcaccacgccgccacactgga | |
| | ctggcgtgctatgccgccgcccagcccctccctgcactactccacactatc | |
| | ctgctcccgatccttccacaacctctctcatcttcccccgtcctatgaggctg | |
| | ctgtgaaatcagaactgaatcgatactcttccctcaagagactggctgaga | |
| | aagatctggatgaagcctacctgaagcgcagacaactggagatgccgc | |
| | gcggaacgctgcccttgcatgcactccggcggcctggcactggaggtgg | |
| | ctaccgtatggatggctggggtggccctgaggagctgggcctggcacca | |
| | gcacccaacccacggcgtgttatgtcccaggagcaccttctgggtgatgg | |
| | tagccgagcttcccgctatgagttcacgttgcctcgagcgcgcctggtgtct | |
| | caggaacacctgctgctgtcctcacctgaggcgcttcgccagagtcgcga | |
| | gcacctgctgtcacccccacgaagtcctgcactgcccccagatcccacc | |
| | acccgggccagcctggctgcctcacactccaacctgctgctagggcctg | |
| | ggggcccccccacacccctgcatgggttgcctccgtcaggcctgcatgcc | |
| | caccatcaccatgcccttcatggctctcctcagccagcctggatgtctgatg | |
| | cgggcgggggtgggggcacactggcccgcaggccacccttccagcgc | |
| | cagggcaccctggagcagctgcagttcattcctgggcaccacctgcccc | |
| | agcacctgcgcactgccagcaagaacgaagtgactgtc | |
| Shisa6 signal peptide | atggcgctgcgccgcctcctgctgccgcctctgctgctgtcgctgctgctgtc | 25 |
| coding sequence | gctcgcgtccctgcacctgccgcccggcgcagacgcc | |
| Shisa7 signal peptide | atgccggccctgctgctgctcgggaccgtcgcgctgctagcctccgcagc | 26 |
| coding sequence | gggcccggcgggggcg | |
| Linker coding sequence | ggcagcggcagcggcagc | 27 |
| HA tag coding sequence | tacccatacgatgttccagattacgct | 28 |
| Coding sequence for | atggcgctgcgccgcctcctgctgccgcctctgctgctgtcgctgct | 29 |
| C3dg-Shisa6-HA with | gctgtcgctcgcgtccctgcacctgccgcccggcgcagacgccg | |
| coding sequence for | ggggagtgcagaaggtggatgtgcctgccgcagaccttagcgaccaag | |
| signal peptide | tgccagacacagactctgagaccagaattatcctgcaagggagcccggt | |
| underlined and bolded, | ggttcagatggctgaagatgctgtggacggggagcggctgaaacacctg | |
| coding sequence for | atcgtgacccccgcaggctgtggggaacagaacatgattggcatgacac | |
| linker italicized and | caacagtcattgcggtacactacctggaccagaccgaacagtgggaga | |
| underlined and coding | agttcggcatagagaagaggcaagaggccctggagctcatcaagaaa | |
| sequence for HA tag | gggtacacccagcagctggccttcaaacagcccagctctgcctatgctgc | |
| underlined | cttcaacaaccggccccccagcacctggctgacagcctacgtggtcaag | |
| | gtcttctctctagctgccaacctcatcgccatcgactctcacgtcctgtgtgg | |
| | ggctgttaaatggttgattctggagaaacagaagccggatggtgtctttcag | |
| | gaggatgggcccgtgattcaccaagaaatgattggtggcttccggaacg | |
| | ccaaggaggcagatgtgtcactcacagccttcgtcctcatcgcactgcag | |
| | gaagccagggacatctgtgaggggcaggtcaatagccttcctgggagc | |
| | atcaacaaggcaggggagtatattgaagccagttacatgaacctgcaga | |
| | gaccatacacagtggccattgctgggtatgccctggccctgatgaacaaa | |
| | ctggaggaaccttacctcggcaagtttctgaacacagccaaagatcgga | |
| | accgctgggaggagcctgaccagcagctctacaacgtagaggccacat | |
| | cctacgccctcctggccctgctgctgctgaaagactttgactctgtgccccct | |
| | gtagtgcgctggctcaatgagcaaagatactacggaggcggctatggct | |
| | ccacccaggctaccttcatggtattccaagccttggcccaatatcaaacag | |
| | atgtccctgaccataaggacttgaacatggatgtgtccttccacctccccag | |
| | ccgtggcagcggcagcggcagcgcccgcggccgctccgggaaccgg | |
| | accctgaacgcgggagccgtcggaggtcggcgtgctgggggcgccctg | |
| | gcccgaggcggccgcgaactaaacagcaccgcccgagcgtccggcgt | |
| | cccggaggcgggcagccggcggggacagtccgcagcagcggcagc | |
| | ggcggcggcggcggcggccagcgcgactgttacttacgagacgtgctg | |
| | gggctactacgacgtgagcggccagtacgacaaggagttcgagtgcaa | |
| | caacagcgagagcggctacctgtactgctgcggcacctgctactatcgctt | |
| | ctgctgcaagaagcgccacgagaagctggaccagcgccagtgcacca | |
| | actaccaaagccccgtatgggtgcagacgcccagcaccaaggtagtgt | |
| | cgccggggcccgagaacaagtacgacccggagaaggacaagacca | |
| | acttcaccgtctacatcacttgcggggtgatcgccttcgtcatcgtggcggg | |
| | cgtcttcgccaaggtctcctatgacaaggcccaccgccctccgcgagag | |
| | atgaacatccacagggctctggctgacattctaagacaacagggaccaa | |
| | tccccatagcacactgtgaaagagaaaccatctcggccatcgatacctct | |
| | cccaaagagaacacgccggtccgatcaacctccaaaaaccactacac | |
| | ccctgtgcgcacagccaagcagactccaggtgatcgtcagtataatcatc | |
| | cgatcttaagcagcgctacccagacccctacacatgagaagccacggat | |
| | gaataacattctgacgtcggccacggaaccctatgacctctccttctcacg | |
| | ctcttaccagaacttagcccacttgcctccatcatatgaatctgcagtgaaa | |
| | accaatccaagcaagtactcgtctctgaagaggctaacggacaaggaa | |
| | gctgatgagtattacatgagaaggaggcacctgccagaccttgcagccc | |
| | gtggtaccctccccctcaatgtcatccagatgtctcaacagaagccacttc | |
| | ctcgagaacggccacgcaggcccatcagggccatgtcccaggacagg | |
| | gtcttgtctccacgtcggggattgccagatgaattcggcatgccctatgacc | |
| | gcatcttgtctgatgaacagctgctctccacagagcgcctgcactcccagg | |
| | acccgttgctgtccccagagaggacagccttcccggagcagtcgctgtcg | |
| | cgggccatctcgcatacggacgtctttgtgtccacgccagtgctggaccgc | |
| | taccgcatgaccaagatgcactcccatcccagtgcctccaataactcctat | |
| | gccaccctgggccagagccagacagcagccaagcgccacgcctttgc | |
| | ctctcgcagacacaacacggtggagcagttacactatatcccaggccatc | |
| | acacctgctacacagccagcaagactgaagtgaccgtgtacccatacg | |
| | atgttccagattacgct* | |
| Coding sequence for | Atggcgctgcgccgcctcctgctgccgcctctgctgctgtcg | 30 |
| Gas6-LG1/LG2-Shisa6- | ctgctgctgtcgctcgcgtccctgcacctgccgcccggcgca | |
| HA coding sequence for | gacgccttcagcgggacccccgtgattagactacgcttcaagagg | |
| signal peptide | cttcagcctaccaggctgctggctgaatttgacttccgcacttttgacc | |
| underlined and bolded, | ctgaaggagtcctcttcttcgctggaggccgttcagacagcacctgg | |
| coding sequence for | attgtcctgggcctaagagctgggcggcttgagctgcagcttcggta | |
| linker italicized and | caatggcgttgggcgcatcaccagcagcgggccaaccatcaacc | |
| underlined and coding | acggcatgtggcaaactatctccgtggaagagctggaacgtaacc | |
| sequence for HA tag | ttgtcatcaaggtcaacaaagatgctgtaatgaagatcgcggtagc | |
| underlined | tggggagctgtttcagctggagaggggcctctatcacctgaatctca | |
| | ccgtgggcggcattcccttcaaggagagtgagctcgtccagccgat | |
| | taaccctcgcctggatgggtgcatgaggagttggaactggctgaac | |
| | ggggaagacagcgccatccaggagacagtcaaggcaaacaca | |
| | aaaatgcagtgcttctctgtgacagaaaggggctccttcttcccggg | |
| | gaatggatttgctacctacaggctcaactacacccgaacatcgctg | |
| | gatgtcggcacggaaaccacctgggaagttaaagttgtggctcgg | |
| | atccgccctgccacggacacgggggtgctgctggcgctggtgggg | |
| | gacgacgatgtcgtccccatctctgtggccctagtcgactaccactct | |
| | acaaagaagctcaagaagcagttggtggtcctggcagttgaggat | |
| | gttgccctggcactgatggaaatcaaggtgtgcgacagccaggaa | |
| | cacacggtcactgtctccctgcgggagggtgaggccaccctagaa | |
| | gtggatggcacaaagggccagagtgaagtgagcactgcccagct | |
| | gcaggagcgactggacacacttaagacacatctgcaaggctctgt | |
| | gcacacctatgttggaggcctgccagaagtatcggtgatttctgcac | |
| | ccgtcactgcgttctaccgcggatgcatgactctggaggtaaacgg | |
| | gaaaatcctggacctggatacggcctcgtacaagcacagtgacat | |
| | cacctcccactcctgcccgcctgtggagcatgccacccccggcagc | |
| | ggcagcggcagcgcccgcggccgctccgggaaccggaccctgaacg | |
| | cgggagccgtcggaggtcggcgtgctgggggcgccctggcccgaggc | |
| | ggccgcgaactaaacagcaccgcccgagcgtccggcgtcccggaggc | |
| | gggcagccggcggggacagtccgcagcagcggcagcggcggcggc | |
| | ggcggcggccagcgcgactgttacttacgagacgtgctggggctactac | |
| | gacgtgagcggccagtacgacaaggagttcgagtgcaacaacagcga | |
| | gagcggctacctgtactgctgcggcacctgctactatcgcttctgctgc | |
| | aagaagcgccacgagaagctggaccagcgccagtgcaccaactaccaaa | |
| | gccccgtatgggtgcagacgcccagcaccaaggtagtgtcgccggggc | |
| | ccgagaacaagtacgacccggagaaggacaagaccaacttcaccgtcta | |
| | catcacttgcggggtgatcgccttcgtcatcgtggcgggcgtcttcgcca | |
| | aggtctcctatgacaaggcccaccgccctccgcgagagatgaacatcca | |
| | cagggctctggctgacattctaagacaacagggaccaatccccatagca | |
| | cactgtgaaagagaaaccatctcggccatcgatacctctcccaaagaga | |
| | acacgccggtccgatcaacctccaaaaaccactacacccctgtgcgcac | |
| | agccaagcagactccaggtgatcgtcagtataatcatccgatcttaagca | |
| | gcgctacccagacccctacacatgagaagccacggatgaataacattct | |
| | gacgtcggccacggaaccctatgacctctccttctcacgctcttaccagaa | |
| | cttagcccacttgcctccatcatatgaatctgcagtgaaaaccaatccaag | |
| | caagtactcgtctctgaagaggctaacggacaaggaagctgatgagtatt | |
| | acatgagaaggaggcacctgccagaccttgcagcccgtggtaccctccc | |
| | cctcaatgtcatccagatgtctcaacagaagccacttcctcgagaacggc | |
| | cacgcaggcccatcagggccatgtcccaggacagggtcttgtctccacgt | |
| | cggggattgccagatgaattcggcatgccctatgaccgcatcttgtctgatg | |
| | aacagctgctctccacagagcgcctgcactcccaggacccgttgctgtcc | |
| | ccagagaggacagccttcccggagcagtcgctgtcgcgggccatctcgc | |
| | atacggacgtctttgtgtccacgccagtgctggaccgctaccgcatgacca | |
| | agatgcactcccatcccagtgcctccaataactcctatgccaccctgggcc | |
| | agagccagacagcagccaagcgccacgcctttgcctctcgcagacaca | |
| | acacggtggagcagttacactatatcccaggccatcacacctgctacaca | |
| | gccagcaagactgaagtgaccgtgtacccatacgatgttccagattacgc | |
| | t* | |
| Coding sequence for | atgccggccctgctgctgctcgggaccgtcgcgctgctagcctcc | 31 |
| C3dg-Shisa7-HA with | gcagcgggcccggcgggggcggggggagtgcagaaggtggatgt | |
| coding sequence for | gcctgccgcagaccttagcgaccaagtgccagacacagactctgagac | |
| signal peptide | cagaattatcctgcaagggagcccggtggttcagatggctgaagatgctg | |
| underlined and bolded, | tggacggggagcggctgaaacacctgatcgtgacccccgcaggctgtg | |
| coding sequence for | gggaacagaacatgattggcatgacaccaacagtcattgcggtacacta | |
| linker italicized and | cctggaccagaccgaacagtgggagaagttcggcatagagaagaggc | |
| underlined and coding | aagaggccctggagctcatcaagaaagggtacacccagcagctggcct | |
| sequence for HA tag | tcaaacagcccagctctgcctatgctgccttcaacaaccggccccccagc | |
| underlined | acctggctgacagcctacgtggtcaaggtcttctctctagctgccaacctca | |
| | tcgccatcgactctcacgtcctgtgtggggctgttaaatggttgattctggag | |
| | aaacagaagccggatggtgtctttcaggaggatgggcccgtgattcacc | |
| | aagaaatgattggtggcttccggaacgccaaggaggcagatgtgtcact | |
| | cacagccttcgtcctcatcgcactgcaggaagccagggacatctgtgag | |
| | gggcaggtcaatagccttcctgggagcatcaacaaggcaggggagtat | |
| | attgaagccagttacatgaacctgcagagaccatacacagtggccattgc | |
| | tgggtatgccctggccctgatgaacaaactggaggaaccttacctcggca | |
| | agtttctgaacacagccaaagatcggaaccgctgggaggagcctgacc | |
| | agcagctctacaacgtagaggccacatcctacgccctcctggccctgctg | |
| | ctgctgaaagactttgactctgtgccccctgtagtgcgctggctcaatgagc | |
| | aaagatactacggaggcggctatggctccacccaggctaccttcatggta | |
| | ttccaagccttggcccaatatcaaacagatgtccctgaccataaggacttg | |
| | aacatggatgtgtccttccacctccccagccgtggcagcggcagcggca | |
| | gccgcccatccaacgacacaagctcagtggccccgggcccgctgcccg | |
| | cgctactcgcgcacctgcggcgcttgaccggggctctggcgggcggcgg | |
| | gagcgcggcaggtaccagcgccaacgccaccaagaccagccccgcg | |
| | agtggcacgggcgcagcggcacgggcgcctcctccggccgagctctgc | |
| | catggctactacgatgtcatgggccagtacgacgccaccttcaactgcag | |
| | caccggctcctaccgcttctgttgtggcacctgccactaccgtttctgctgcg | |
| | agcaccgccacatgcgcctggcgcaggcctcctgctccaactacgacac | |
| | gccacgctgggccaccacgcccccgccgctggctggaggcgccgggg | |
| | gcgctgggggtgcggggggggaccagggccgggccaggcagggtg | |
| | gctggaagggggccgggccgggggcgctgggggacgtgggggagag | |
| | ggcccagggggcagcacagcctacgtggtgtgcggagtcatcagtttcg | |
| | ccctggcggtgggcgtcggtgccaaagtggccttcagcaaggcgtcacg | |
| | tgcacccagggcgcaccgggagatcaacgtgcccagagctctcgtgga | |
| | tattctcaggcatcaagcaggacctgcaacccgcccggaccgggccag | |
| | aagcagttctctgaccccagggctgggaggcccagacagcatggcccc | |
| | aaggacacccaagaacctttacaacaccatgaagccctccaacctcgat | |
| | aacctgcactacaacgtcaacagccccaagcaccacgccgccacactg | |
| | gactggcgtgctatgccgccgcccagcccctccctgcactactccacact | |
| | atcctgctcccgatccttccacaacctctctcatcttcccccgtcctatgagg | |
| | ctgctgtgaaatcagaactgaatcgatactcttccctcaagagactggctg | |
| | agaaagatctggatgaagcctacctgaagcgcagacaactggagatgc | |
| | cgcgcggaacgctgcccttgcatgcactccggcggcctggcactggagg | |
| | tggctaccgtatggatggctggggggccctgaggagctgggcctggcac | |
| | cagcacccaacccacggcgtgttatgtcccaggagcaccttctgggtgat | |
| | ggtagccgagcttcccgctatgagttcacgttgcctcgagcgcgcctggtg | |
| | tctcaggaacacctgctgctgtcctcacctgaggcgcttcgccagagtcgc | |
| | gagcacctgctgtcacccccacgaagtcctgcactgcccccagatccca | |
| | ccacccgggccagcctggctgcctcacactccaacctgctgctagggcct | |
| | gggggcccccccacacccctgcatgggttgcctccgtcaggcctgcatgc | |
| | ccaccatcaccatgcccttcatggctctcctcagccagcctggatgtctgat | |
| | gcgggcgggggtgggggcacactggcccgcaggccacccttccagcg | |
| | ccagggcaccctggagcagctgcagttcattcctgggcaccacctgccc | |
| | cagcacctgcgcactgccagcaagaacgaagtgactgtctacccatacg | |
| | atgttccagattacgct* | |
| Coding sequence for | Atgccggccctgctgctgctcgggaccgtcgcgctgctagcctcc | 32 |
| Gas6-LG1/LG2-Shisa7- | gcagcgggcccggcgggggcgttcagcgggacccccgtgatta | |
| HA coding sequence for | gactacgcttcaagaggcttcagcctaccaggctgctggctgaattt | |
| signal peptide | gacttccgcacttttgaccctgaaggagtcctcttcttcgctggaggc | |
| underlined and bolded, | cgttcagacagcacctggattgtcctgggcctaagagctgggcggc | |
| coding sequence for | ttgagctgcagcttcggtacaatggcgttgggcgcatcaccagcag | |
| linker italicized and | cgggccaaccatcaaccacggcatgtggcaaactatctccgtgga | |
| underlined and coding | agagctggaacgtaaccttgtcatcaaggtcaacaaagatgctgta | |
| sequence for HA tag | atgaagatcgcggtagctggggagctgtttcagctggagaggggc | |
| underlined | ctctatcacctgaatctcaccgtgggcggcattcccttcaaggagag | |
| | tgagctcgtccagccgattaaccctcgcctggatgggtgcatgagg | |
| | agttggaactggctgaacggggaagacagcgccatccaggaga | |
| | cagtcaaggcaaacacaaaaatgcagtgcttctctgtgacagaaa | |
| | ggggctccttcttcccggggaatggatttgctacctacaggctcaact | |
| | acacccgaacatcgctggatgtcggcacggaaaccacctgggaa | |
| | gttaaagttgtggctcggatccgccctgccacggacacgggggtgc | |
| | tgctggcgctggtgggggacgacgatgtcgtccccatctctgtggcc | |
| | ctagtcgactaccactctacaaagaagctcaagaagcagttggtg | |
| | gtcctggcagttgaggatgttgccctggcactgatggaaatcaaggt | |
| | gtgcgacagccaggaacacacggtcactgtctccctgcgggagg | |
| | gtgaggccaccctagaagtggatggcacaaagggccagagtga | |
| | agtgagcactgcccagctgcaggagcgactggacacacttaaga | |
| | cacatctgcaaggctctgtgcacacctatgttggaggcctgccaga | |
| | agtatcggtgatttctgcacccgtcactgcgttctaccgcggatgcat | |
| | gactctggaggtaaacgggaaaatcctggacctggatacggcctc | |
| | gtacaagcacagtgacatcacctcccactcctgcccgcctgtggag | |
| | catgccacccccggcagcggcagcggcagccgcccatccaacgaca | |
| | caagctcagtggccccgggcccgctgcccgcgctactcgcgcacctgcg | |
| | gcgcttgaccggggctctggcgggcggcgggagcgcggcaggtacca | |
| | gcgccaacgccaccaagaccagccccgcgagtggcacgggcgcagc | |
| | ggcacgggcgcctcctccggccgagctctgccatggctactacgatgtca | |
| | tgggccagtacgacgccaccttcaactgcagcaccggctcctaccgcttc | |
| | tgttgtggcacctgccactaccgtttctgctgcgagcaccgccacatgcgc | |
| | ctggcgcaggcctcctgctccaactacgacacgccacgctgggccacca | |
| | cgcccccgccgctggctggaggcgccgggggcgctgggggtgcgggt | |
| | gggggaccagggccgggccaggcagggtggctggaagggggccgg | |
| | gccgggggcgctgggggacgtgggggagagggcccagggggcagc | |
| | acagcctacgtggtgtgcggagtcatcagtttcgccctggcggtgggcgtc | |
| | ggtgccaaagtggccttcagcaaggcgtcacgtgcacccagggcgcac | |
| | cgggagatcaacgtgcccagagctctcgtggatattctcaggcatcaagc | |
| | aggacctgcaacccgcccggaccgggccagaagcagttctctgacccc | |
| | agggctgggaggcccagacagcatggccccaaggacacccaagaac | |
| | ctttacaacaccatgaagccctccaacctcgataacctgcactacaacgt | |
| | caacagccccaagcaccacgccgccacactggactggcgtgctatgcc | |
| | gccgcccagcccctccctgcactactccacactatcctgctcccgatccttc | |
| | cacaacctctctcatcttcccccgtcctatgaggctgctgtgaaatcagaac | |
| | tgaatcgatactcttccctcaagagactggctgagaaagatctggatgaa | |
| | gcctacctgaagcgcagacaactggagatgccgcgcggaacgctgccc | |
| | ttgcatgcactccggcggcctggcactggaggtggctaccgtatggatgg | |
| | ctggggtggccctgaggagctgggcctggcaccagcacccaacccacg | |
| | gcgtgttatgtcccaggagcaccttctgggtgatggtagccgagcttcccg | |
| | ctatgagttcacgttgcctcgagcgcgcctggtgtctcaggaacacctgct | |
| | gctgtcctcacctgaggcgcttcgccagagtcgcgagcacctgctgtcac | |
| | ccccacgaagtcctgcactgcccccagatcccaccacccgggccagcc | |
| | tggctgcctcacactccaacctgctgctagggcctgggggcccccccaca | |
| | cccctgcatgggttgcctccgtcaggcctgcatgcccaccatcaccatgcc | |
| | cttcatggctctcctcagccagcctggatgtctgatgcgggcgggggggg | |
| | ggcacactggcccgcaggccacccttccagcgccagggcaccctgga | |
| | gcagctgcagttcattcctgggcaccacctgccccagcacctgcgcactg | |
| | ccagcaagaacgaagtgactgtctacccatacgatgttccagattacgct* | |

The provided nucleic acids herein may in various aspects be provided in an expression vector. Expression vectors are specific vectors (described in more detail below) that are capable of directing the expression of nucleic acids to which they are operatively linked. Such vectors are referred to herein as “recombinant expression vectors”, or more simply “expression vectors”, which serve equivalent functions. Accordingly, provided herein are one or more expression vectors comprising the nucleic acids herein. In some aspects, a set of expression vectors is provided comprising a first expression vector comprising the first nucleic acid (e.g., encoding a first fusion protein provided herein) and a second expression vector comprising the second nucleic acid (e.g., encoding a second fusion protein). This allows for delivery and expression of the first and second fusion protein into separate cells as described further below.

In any of the aspects herein, the nucleic acids and/or expression vectors may further comprise one or more transcription and/or translation control elements. Depending on the host and system utilized, any of a number of suitable transcription and translation control elements, including constitutive and inducible promoters, transcription enhancer elements, transcription terminators, etc. can be used in the expression vector and/or nucleic acids herein. The transcription and translation control element can be tissue-specific or ubiquitous and can be constitutive or inducible, depending on the pattern of the gene expression desired. The transcription and translation control element can be native or foreign and can be a natural or a synthetic sequence. By foreign, it is intended that the transcriptional initiation region is not found in the wild-type host into which the transcriptional initiation region is introduced.

Suitable transcription and translation control elements include promoters, enhancers, and/or transcriptional termination signals.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter, CAG promoter). In some cases, the promoter can be a spatially restricted and/or temporally restricted promoter (e.g., a tissue specific promoter, a cell type specific promoter, etc.). In some aspects, the promoter can be CRE-dependent.

The promoter can be chosen so that it will function in the target cell(s) of interest. Tissue-specific promoters refer to promoters that have activity in only certain cell types. The use of a tissue-specific promoter in a nucleic acid expression cassette can restrict unwanted transgene expression in the unaffected tissues as well as facilitate persistent transgene expression by escaping from transgene induced host immune responses. Tissue specific promoters include, but are not limited to, neuron-specific promoters, muscle-specific promoters, liver-specific promoters, skeletal muscle-specific promoters, and heart-specific promoters. Examples of neuron-specific promoters include, but are not limited to, Synapsin, CAMKIIa, tyrosine hydroxylase, thymus cell antigen 1, neuron-specific enolase and other natural and synthetic neuron-specific promoters. In one embodiment, the promoter comprises a synapsin or CAMKIIa promoter.

In some aspects, the promoter is an inducible promoter. Inducible promoters refer to promoters that can be regulated by positive or negative control. Factors that can regulate an inducible promoter include, but are not limited to, chemical agents (e.g., the metallothionein promoter or a hormone inducible promoter), temperature, and light.

In some aspects, the promoter is a neuron-specific inducible promoter using a Cre-Lox system. For example, in some aspects an AAV-double inverted repeat (DIO) construct can be delivered into a Cre-expressing animal. This system results in the expression of a Cre endonuclease in a cell type dictated by the DIO construct. When Cre is expressed it cleaves a target nucleic acid at Lox sites, which can be engineered to surround a regulatory element of a gene of interest. Selective activation of Cre, therefore, results in selective expression of the gene of interest in a target cel. In the disclosure herein, an exemplary promoter that may be used in this way is a Syn1-DIO promoter, which links the DIO system to a synapsin 1 promoter.

In other aspects, the promoter can be a constitutive promoter. Constitutive promoters refer to promoters that allow for continual transcription of its associated gene. Constitutive promoters are always active and can be used to express genes in a wide range of cells and tissues, including, but not limited to, the liver, kidney, skeletal muscle, cardiac muscle, smooth muscle, diaphragm muscle, brain, spinal cord, endothelial cells, intestinal cells, pulmonary cells (e.g., smooth muscle or epithelium), peritoneal epithelial cells and fibroblasts. Examples of constitutive promoters include, but are not limited to, a CMV major immediate-early enhancer/chicken beta-actin promoter, a cytomegalovirus (CMV) major immediate-early promoter, a simian vacuolating virus 40 (SV40) promoter, an AmpR promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous promoters. In some embodiments, the constitutively active promoter is selected from the group consisting of human β-actin, human elongation factor-1a, chicken β-actin combined with cytomegalovirus early enhancer, cytomegalovirus (CMV), simian virus 40, or herpes simplex virus thymidine kinase.

The tissue-specific promoters can be operably linked to one or more (e.g., 2, 3, 4, 5, 6, 7, or 8) enhancer elements (e.g., a neuron-specific promoter fused to a cytomegalovirus enhancer) or combined to form a tandem promoter (e.g., neuron-specific/constitutive tandem promoter). When two or more tissue-specific promoters are present, the isolated nucleic acid can be targeted to two or more different tissues at the same time.

As discussed above, a disclosed promoter can be an endogenous promoter. Endogenous refers to a disclosed promoter or disclosed promoter/enhancer that is naturally linked with its gene. In an aspect, a disclosed endogenous promoter can generally be obtained from a non-coding region upstream of a transcription initiation site of a gene (such as, for example, a disclosed phosphorylase kinase, phosphorylase, or some other enzyme involved in the glycogen metabolic pathway). In an aspect, a disclosed endogenous promoter can be used for constitutive and efficient expression of a disclosed transgene (e.g., a nucleic acid sequence encoding a polypeptide capable of preventing glycogen accumulation and/or degrading accumulated glycogen). In an aspect, a disclosed endogenous promoter can be an endogenous promoter/enhancer.

As discussed above, a disclosed promoter can be an exogenous promoter. Exogenous (or heterologous) refers to a disclosed promoter or a disclosed promoter/enhancer that can be placed in juxtaposition to a gene by means of molecular biology techniques such that the transcription of that gene can be directed by the linked promoter or linked promoter/enhancer.

An enhancer element is a nucleic acid sequence that functions to enhance transcription. As used herein, the terms “enhance” and “enhancement” with respect to nucleic acid expression or polypeptide production, refers to an increase and/or prolongation of steady-state levels of the indicated nucleic acid or polypeptide, e.g., by at least about 2%, 5%, 10%, 15%, 20%, 25%, 40%, 50%, 60%, 75%, 2-fold, 2.5-fold, 3-fold, 5-fold, 10-fold, 15-fold, 20-fold, 30-fold, 50-fold, 100-fold or more. As used herein, the term “intron” refers to nucleic acid sequences that can enhance transgene expression. An intron can also be a part of the nucleic acid expression cassette or positioned downstream or upstream of the expression cassette in the expression vector. Introns can include, but are not limited to, the SV40 intron, EF-1alpha gene intron 1, or the MVM intron. In some embodiments, the nucleic acid expression cassettes do not contain an intron. Representative enhancer elements that can be used herein include any enhancer elements normally associated with a synaptic protein gene (e.g., a Shisa6 or Shisa7 gene).

In other aspects, the nucleic acids and/or expression vectors according to the present disclosure can further comprise a transcriptional termination signal. A transcriptional termination signal is a nucleic acid sequence that marks the end of a gene during transcription. Examples of a transcriptional termination signal include, but are not limited to, bovine growth hormone polyadenylation signal (BGHpA), Simian virus 40 polyadenylation signal (Sv40 PolyA), and a synthetic polyadenylation signal. A polyadenylation sequence can comprise the nucleic acid sequence AATAAA. In some embodiments, the nucleic acid encoding the fusion protein comprises a FLAG tag at the C-terminus.

In any of the foregoing or related aspects, the nucleic acids disclosed herein may be “codon optimized” to ensure expression in a target cell or organism. As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

In various aspects, the expression vectors provided herein may be packaged into a delivery vector (i.e., as a recombinant expression vector). In this case, the term “vector” refers to a nucleic acid molecule capable of transporting another nucleic acid to which it has been linked. It will be apparent to those skilled in the art that any suitable vector can be used to deliver the isolated nucleic acids of the disclosure to the target cell(s) or subject of interest. The choice of delivery vector can be made based on a number of factors known in the art, including age and species of the target host, in vitro vs. in vivo delivery, level and persistence of expression desired, intended purpose (e.g., for therapy or enzyme production), the target cell or organ, route of delivery, size of the isolated nucleic acid, safety concerns, and the like.

In accord with any of the foregoing or related aspects, the expression vectors can comprise one or more further elements (e.g., transcription and/or translation control elements described above) that enable expression of nucleic acids of interest in a target cell or organism. The expression vectors can be viral or non-viral as described further below. Suitable expression vectors that are known in the art and that can be used to deliver, and optionally, express the isolated nucleic acids of the disclosure (e.g., viral and non-viral vectors), including, virus vectors (e.g., retrovirus, adenovirus, AAV, lentiviruses, or herpes simplex virus), lipid vectors, poly-lysine vectors, synthetic polyamino polymer vectors that are used with nucleic acid molecules, such as a plasmid, and the like. In some embodiments, the non-viral vector can be a polymer-based vector (e.g., polyethylenimine (PEI), chitosan, poly (DL-Lactide) (PLA), or poly (DL-lactidie-co-glycoside) (PLGA), dendrimers, polymethacrylate) a peptide-based vector, a lipid nanoparticle, a solid lipid nanoparticle, or a cationic lipid based vector.

Other types of vectors include “plasmids”, which are circular double-stranded DNA loops into which additional nucleic acid segments can be ligated and viral vectors wherein additional nucleic acid segments can be ligated into the viral genome and which comprises the vector genome (e.g., viral DNA) packaged within a virion. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some embodiments, the nucleic acid encoding for a fusion protein herein can be incorporated into a recombinant viral vector. As used herein, the term “viral vector” refers to a virus (e.g., AAV) particle that functions as a nucleic acid delivery vehicle, and which comprises the vector genome (e.g., viral DNA) packaged within a virion. Alternatively, in some contexts, the term “vector” is used to refer to the vector genome/viral DNA alone.

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia).

In some aspects, the vector is a recombinant viral vector suitable for gene therapy. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picornaviridae; Plasmaviridae; Prodoviridae; Polydnaviridae; Potexvirus group; Potyvirus; Poxyiridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and plant virus satellites.

In some embodiments, the recombinant viral vector is selected from the group consisting of adenoviruses, Adeno-associated viruses (AAV) (e.g., AAV serotypes and genetically modified AAV variants), a herpes simplex viruses (e.g., e.g., HSV-1, HSV), a retrovirus vector (e.g., MMSV, MSCV), a lentivirus vector (HIV-1, HIV-2), and alphavirus vector (e.g., SFV, SIN, VEE, M1), a flavivirus vector (e.g., Kunjin, West Nile, Dengue virus), a rhabdovirus vector (e.g., Rabies, VSV), a measles virus vector (e.g., MV-Edm), a Newcastle disease virus vector, a poxvirus vector (VV), or a picornavirus vector (e.g., Coxsackievirus). The recombinant viral vector of the present disclosure includes any type of viral vector that is capable of packaging and delivering the fusion proteins or viral vectors that can be designed engineered and generated by methods known in the art.

In some embodiments, the delivery vector or expression vector is an adenovirus vector. The term “adenovirus” as used herein encompasses all adenoviruses, including the Mastadenovirus and Aviadenovirus genera.

The various regions of the adenovirus genome have been mapped and are understood by those skilled in the art. The genomic sequences of the various Ad serotypes, as well as the nucleotide sequence of the particular coding regions of the Ad genome, are known in the art and may be accessed from GenBank and NCBI (see, e.g., GenBank Accession Nos. J0917, M73260, X73487, AF108105, L19443, NC 003266 and NCBI Accession Nos. NC 001405, NC 001460, NC 002067, NC 00454).

A recombinant adenovirus (rAd) vector genome can comprise the adenovirus terminal repeat sequences and packaging signal. An “adenovirus particle” or “recombinant adenovirus particle” comprises an adenovirus vector genome or recombinant adenovirus vector genome, respectively, packaged within an adenovirus capsid. Generally, the adenovirus vector genome is most stable at sizes of about 28 kb to 38 kb (approximately 75% to 105% of the native genome size). In the case of an adenovirus vector containing large deletions and a relatively small transgene, “stutter DNA” can be used to maintain the total size of the vector within the desired range by methods known in the art.

The genome of an adenovirus can be manipulated such that it encodes and expresses a gene product of interest but is inactivated in terms of its ability to replicate in a normal lytic viral life cycle. Suitable adenoviral vectors derived from the adenovirus strain Ad type 5 (Ad5) or other strains of adenovirus (e.g., Ad2, Ad3, Ad7, etc.) are known to those skilled in the art.

In some embodiments, the viral vector comprises a recombinant Adeno-Associated Viruses (AAV). AAV are parvoviruses and have small icosahedral virions and can contain a single stranded DNA molecule about 4.7 kb (e.g., about 4.5 kb, 4.6 kb, 4.8 kb, 4.9 kb, or 5.0 kb) or less in size. The viruses contain either the sense or antisense strand of the DNA molecule and either strand is incorporated into the virion. Two open reading frames encode a series of Rep and Cap polypeptides. Rep polypeptides (e.g., Rep50, Rep52, Rep68 and Rep78) are involved in replication, rescue and integration of the AAV genome, although significant activity may be observed in the absence of all four Rep polypeptides. The Cap proteins (e.g., VP1, VP2, VP3) form the virion capsid. Flanking the rep and cap open reading frames at the 5′ and 3′ ends of the genome are inverted terminal repeats (ITRs). Typically, in recombinant AAV (rAAV) vectors, the entire rep and cap coding regions are excised and replaced with a transgene of interest.

Recombinant AAV vectors generally require only the inverted terminal repeat(s) (ITR(s)) in cis to generate virus. All other viral sequences are dispensable and may be supplied in trans. Typically, the rAAV vector genome will only retain the one or more ITR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the present disclosure, the rAAV vector genome comprises at least one terminal repeat (ITR) sequence (e.g., AAV TR sequence), optionally two ITRs (e.g., two AAV ITRs), which typically will be at the 5′ and 3′ ends of the vector genome and flank the heterologous nucleic acid sequence, but need not be contiguous thereto. The ITRs can be the same or different from each other.

The term “inverted terminal repeat” or “ITR” is used equivalently herein with the term “terminal repeat” or “TR” and includes any viral terminal repeat or synthetic sequence that forms a hairpin structure and functions as an inverted terminal repeat (i.e., mediates the desired functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a ITR, which can further be modified by truncation, substitution, deletion, insertion and/or addition. Further, the ITR can be partially or completely synthetic, such as the “double-D sequence.”

An “AAV inverted terminal repeat” or “AAV ITR” may be from any AAV, including but not limited to serotypes 1, 2, 3, 4, 5, 6, 7, 8, 9, 10 or 11 or any other AAV now known or later discovered. An AAV terminal repeat need not have the native terminal repeat sequence (e.g., a native AAV ITR sequence may be altered by insertion, deletion, truncation and/or missense mutations), as long as the terminal repeat mediates the desired functions, e.g., replication, virus packaging, integration, and/or provirus rescue, and the like. In some embodiments, the vector comprises flanking ITRs derived from the AAV2 genome.

Wild-type AAV can integrate their DNA into non-dividing cells and exhibit a high frequency of stable integration into human chromosome 19. A rAAV vector genome will typically comprise the AAV terminal repeat sequences and packaging signal.

An “AAV particle” or “rAAV particle” comprises an AAV vector genome or rAAV vector genome, respectively, packaged within an AAV capsid. The AAV rep/cap genes can be expressed on a single plasmid. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extrachromosomal elements, designated as an “EBV based nuclear episome,” see Margolski (1992) Curr. Top. Microbiol. Immun. 158:67). The AAV rep and cap genes may be from any AAV serotype for which recombinant virus can be derived and may be from a different AAV serotype than the rAAV genome ITRs.

However, the rAAV vector itself need not contain AAV genes encoding the capsid (cap) and Rep proteins. In particular embodiments of the disclosure, the rep and/or cap genes are deleted from the AAV genome. In a representative embodiment, the rAAV vector retains only the terminal AAV sequences (ITRs) necessary for integration, excision, and replication.

Sources for the AAV capsid genes can include naturally isolated serotypes, including but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7, as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In particular embodiments, the AAV capsids are chimeras either created by capsid evolution or by rational capsid engineering from the naturally isolated AAV variants to capture desirable serotype features such as enhanced or specific tissue tropism and host immune response escape, including but not limited to AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS., AAV-PHP.B, AAV-PHP.eB, and AAV-PHP.S.

Accordingly, when referring herein to a specific AAV capsid protein (e.g., an AAV2, AAV3, AAV4, AAV5, AAV6, AAV7, AAV8, AAV9, AAV10, AAV1 or AAV12 capsid protein) it is intended to encompass the native capsid protein as well as capsid proteins that have alterations other than the modifications of the invention. Such alterations include substitutions, insertions and/or deletions.

some embodiments, the recombinant AAV vectors are selected from the group consisting of AAV7, AAV1, AAV10, AAV8, or AAV9. In certain embodiments, the recombinant AAV vector comprises AAV9 due to its ability to easily cross the blood-brain barrier.

In some embodiments, the recombinant viral vectors (e.g., rAAV) according to the present disclosure generally comprise, consist of, or consist essentially of one or more of the following elements: (1) an Inverted Terminal Repeat sequence (ITR); (2) a promoter (e.g., a neuron specific promoter); (3) a nucleic acid encoding a gene or protein of interest (e.g., a nucleic acid encoding the fusion protein provided herein); (4) a transcription terminator (e.g., a polyadenylation signal); and (5) a flanking Inverted Terminal Repeat sequence (ITR).

In some embodiments, the recombinant viral vectors can comprise a linker sequence. The term “linker sequence” as used herein refers to a nucleic acid sequence that encodes a short polypeptide sequence. A linker sequence can comprise at least 6 nucleotide sequences, at least 15 nucleotides, 27 nucleotides, or at least 30 nucleotides. In some embodiments, the linker sequence has 6 to 27 nucleotides. In other embodiments, the linker sequence has 6 nucleotides, 15 nucleotides, and/or 27 nucleotides. A linker sequence can be used to connect various encoded elements in the vector constructs. For example, a transgene and Myc tag can be operably linked via a linker, or a Myc tag and FLAG can be operably linked via a linker or a FLAG tag and mCherry tag can be operably linked via a linker. Alternatively, the vector elements can be directly linked (e.g., not via a linker).

In some embodiments, the AAV vectors are pseudotyped, which refers to the practice of creating hybrids of certain AAV strains to be able to refine the interaction with desired target cells. The hybrid AAV can be created by taking a capsid from one strain and the genome from another strain. For example, AAV2/5, a hybrid with the genome of AAV2 and the capsid of AAV5, can be used to achieve more accuracy and range in brain cells than AAV2 would be able to achieve unhybridized. Production of pseudotyped rAAV is disclosed in, for example, WO01/83692.

Other types of rAAV variants, for example rAAV with capsid mutations, are also contemplated. See, for example, Marsic et al., Molecular Therapy, 22 (11): 1900-1909 (2014). It is understood that the nucleotide sequences of the genomes of various AAV serotypes are known in the art.

Examples of recombinant AAV that can be constructed to comprise the nucleic acid molecules of the disclosure are set out in International Patent Application No. PCT/US2012/047999 (WO 2013/016352) incorporated by reference herein in its entirety.

Any suitable method known in the art can be used to produce AAV vectors. In one particular method, AAV stocks can be produced by co-transfection of a rep/cap vector plasmid encoding AAV packaging functions and the vector plasmid containing the recombinant AAV genome into human cells infected with the helper adenovirus. General principles of recombinant AAV production are reviewed in, for example, Carter, 1992, Current Opinions in Biotechnology, 1533-539; and Muzyczka, (1992) Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. Nos. 5,173,414; 5,658,776; WO 95/13392; WO 96/17947; WO 97/09441; WO 97/08298; WO 97/21825; WO 97/06243; WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to recombinant AAV production.

The recombinant viral vectors (e.g., rAAV) may be purified by methods standard in the art such as by column chromatography or cesium chloride gradients. Methods for purifying recombinant viral vectors from helper virus are known in the art.

The nucleic acid encoding the fusion protein can be provided to the cell using any method known in the art. For example, the template can be supplied by a non-viral (e.g., plasmid) or viral vector.

The AAV rep and/or cap genes can alternatively be provided by a packaging cell that stably expresses the genes. A method of generating a packaging cell is to create a cell line that stably expresses all the necessary components for viral (e.g., AAV) particle production. For example, in one embodiment, a plasmid (or multiple plasmids) comprising a viral rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

In one embodiment, packaging cells can be stably transformed cancer cells such as Hela cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In still further embodiments, the delivery vectors are a hybrid Ad-AAV delivery vector. Briefly, the hybrid Ad-AAV vector comprises an adenovirus vector genome comprising adenovirus (i) 5′ and 3′ cis-elements for viral replication and encapsidation and, further, (ii) a recombinant AAV vector genome comprising the AAV 5′ and 3′ inverted terminal repeats (ITRs), an AAV packaging sequence, and a heterologous sequence(s) flanked by the AAV ITRs, where the recombinant AAV vector genome is flanked by the adenovirus 5′ and 3′ cis-elements. The adenovirus vector genome can further be deleted, as described above.

Another vector for use in the present disclosure comprises Herpes Simplex Virus (HSV). HSV can be modified for the delivery of transgenes to cells by producing a vector that exhibits only the latent function for long-term gene maintenance. HSV vectors are useful for nucleic acid delivery because they allow for a large DNA insert of up to or greater than 20 kilobases; they can be produced with extremely high titers; and they have been shown to express transgenes for a long period of time in the central nervous system as long as the lytic cycle does not occur.

Herpes virus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al. (1999) Gene Therapy 6:986 and WO 00/17377.

In other embodiments of the present disclosure, the delivery vector of interest is a retrovirus. Retroviruses normally bind to a species-specific cell surface receptor, e.g., CD4 (for HIV); CAT (for MLV-E; ecotropic Murine leukemic virus E); RAM1/GLVR2 (for murine leukemic virus-A; MLV-A); GLVR1 (for Gibbon Ape leukemia virus (GALV) and Feline leukemia virus B (FeLV-B)). The development of specialized cell lines (termed “packaging cells”) which produce only replication-defective retroviruses has increased the utility of retroviruses for gene therapy, and defective retroviruses are characterized for use in gene transfer for gene therapy purposes. A replication-defective retrovirus can be packaged into virions which can be used to infect a target cell through the use of a helper virus by standard techniques.

Yet another suitable vector is a lentiviral vector. Lentiviruses are a subtype of retroviruses but they have the unique ability to infect non-dividing cells, and therefore can have a ride range of potential applications.

Yet another suitable vector is a poxvirus vector. These viruses contain more than 100 proteins. Extracellular forms of the virus have two membranes while intracellular particles only have an inner membrane. The outer surface of the virus is made up of lipids and proteins that surround the biconcave core. Poxviruses are very complex antigenically, inducing both specific and cross-reacting antibodies after infection. Poxvirus can infect a wide range of cells. Poxvirus gene expression is well studied due to the interest in using vaccinia virus as a vector for expression of transgenes.

In another representative embodiment, the nucleic acid sequence encoding the fusion protein is provided by a replicating rAAV virus. In still other embodiments, an AAV provirus comprising the nucleic acid sequence encoding the fusion protein can be stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

In addition to viral transfer methods, such as those illustrated above, non-viral methods can also be employed. Many non-viral methods of gene transfer rely on normal mechanisms used by mammalian cells for the uptake and intracellular transport of macromolecules. In particular embodiments, non-viral delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present disclosure. Naked plasmids can be introduced into cells by injection into the tissue. Expression can extend over many months. Cationic lipids can aid in introduction of DNA into some cells in culture. Injection of cationic lipid plasmid DNA complexes into the circulation of mice can result in expression of the DNA in organs (e.g., the lung). One advantage of plasmid DNA is that it can be introduced into non-replicating cells.

In a representative embodiment, a nucleic acid molecule (e.g., a plasmid) can be entrapped in a lipid particle bearing positive changes on its surface and, optionally, tagged with antibodies against cell surface antigens of the target tissue.

Liposomes that consist of amphiphilic cationic molecules are useful non-viral vectors for nucleic acid delivery in vitro and in vivo. The positively charged liposomes are believed to complex with negatively charged nucleic acids via electrostatic interactions to form lipid: nucleic acid complexes. The lipid: nucleic acid complexes have several advantages as gene transfer vectors. Unlike viral vectors, the lipid: nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency.

Amphiphilic cationic lipid: nucleic acid complexes can be used for in vivo transfection both in animals and in humans and can be prepared to have a long shelf-life.

In addition, vectors according to the present disclosure can be used in diagnostic and screening methods, whereby a nucleic acid encoding the fusion protein is transiently or stably expressed in a cell culture system, or alternatively, a transgenic animal model screening method, whereby a nucleic acid of interest is transiently or stably expressed in a cell culture system, or alternatively, a transgenic animal model.

The vectors of the present disclosure can also be used for various non-therapeutic purposes, including but not limited to use in protocols to assess gene targeting, clearance, transcription, translation, etc., as would be apparent to one skilled in the art. The vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

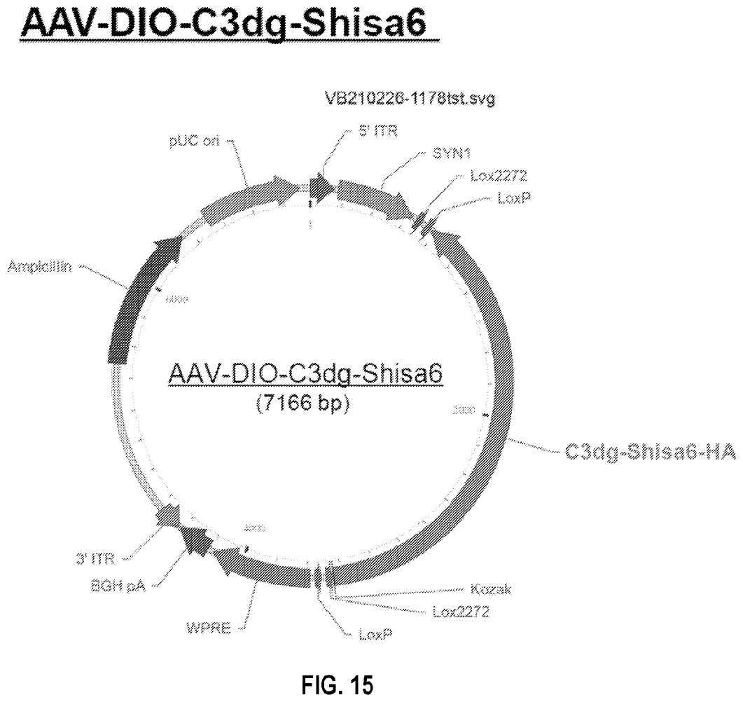

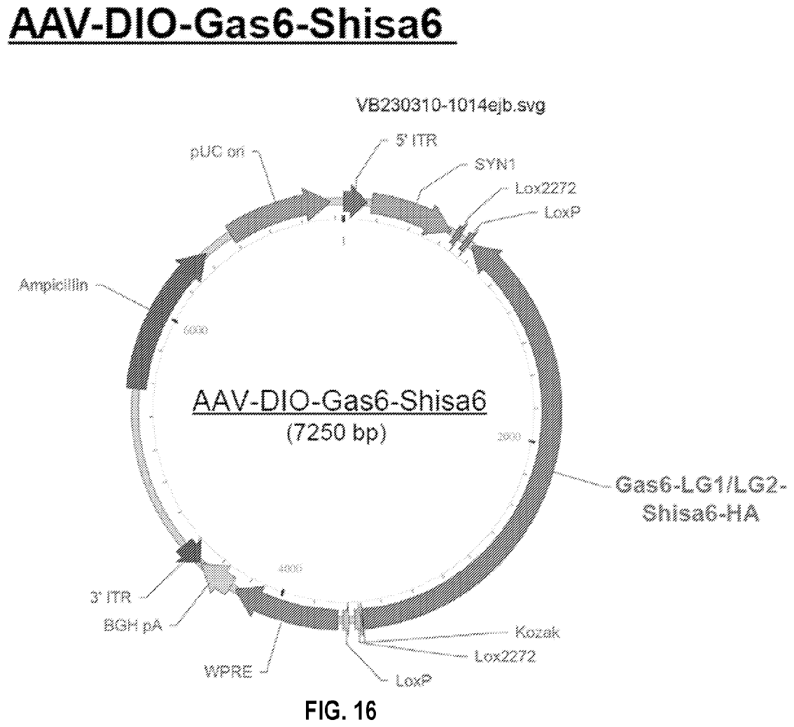

In accord with the foregoing aspects, two expression vectors for directing expression of C3dg-Shisa6 or Gas6-Shisa6 are depicted in FIG. 15 (C3dg-Shisa6, SEQ ID NO: 33) and FIG. 16 (Gas6-Shisa6, SEQ ID NO: 34). These vectors are described further in the Examples below.

### (d) Pharmaceutical Compositions

Another aspect of the present disclosure provides a composition and/or pharmaceutical formulation comprising, consisting, or consisting essentially of one or more a nucleic acids or expression vectors provided herein. Regarding the fusion protein systems, provided herein, various methods provided below involve delivering at least two separate nucleic acids (e.g., a nucleic acid encoding a “first” fusion protein and a nucleic acid encoding a “second” fusion protein) to at least two different cells (i., a presynaptic neuron and a postsynaptic neuron). Accordingly, in various aspects, the disclosure herein provides for a set of two compositions and/or pharmaceutical formulations each comprising one of a set of two nucleic acids/expression vectors expressing the first or second fusion protein.

In some embodiments, compositions of the present disclosure comprise, consist of, or consist essentially of a recombinant viral vector (e.g., rAAV) and/or a pharmaceutically acceptable carrier and/or excipient, and, optionally, other medicinal agents, pharmaceutical agents, stabilizing agents, buffers, carriers, adjuvants, diluents, etc. For injection, the carrier will typically be a liquid. For other methods of administration, the carrier can be either solid or liquid. For inhalation administration, the carrier will be respirable, and optionally can be in solid or liquid particulate form.

By “pharmaceutically acceptable” it is meant a material that is not biologically or otherwise undesirable, i.e., the material can be administered to a subject along with the isolated nucleic acid or vector without causing any undesirable biological effects such as toxicity. Thus, such a pharmaceutical composition can be used, for example, in transfection of a cell ex vivo or in administering an isolated nucleic acid or vector directly to a subject.

The compositions can also comprise other ingredients such as diluents and adjuvants. Acceptable carriers, diluents and adjuvants are nontoxic to recipients and are preferably inert at the dosages and concentrations employed, and can include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

The pharmaceutical carriers, diluents or excipients suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some embodiments, sterile injectable solutions are prepared by incorporating the recombinant viral vector (e.g., rAAV) in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of recombinant viral vector (e.g., rAAV) as a free acid (DNA contains acidic phosphate groups) or a pharmacologically acceptable salt can be prepared in water suitably mixed with a surfactant such as hydroxpropylcellulose. A dispersion of recombinant viral vector (e.g., rAAV) can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

In an aspect, a disclosed pharmaceutical formulation can regulate, restore, normalize, and/or maintain one or more liver enzymes and/or metabolites. Liver enzyme and/or metabolites can comprise Alanine transaminase (ALT), Aspartate transaminase (AST), Alkaline phosphatase (ALP), Albumin and total protein, Bilirubin, Gamma-glutamyltransferase (GGT), L-lactate dehydrogenase (LD), Prothrombin time (PT), or any combination thereof. In an aspect, a disclosed pharmaceutical formulation can regulate, restore, normalize, and/or maintain one or more urine enzymes and/or metabolites (such as, for example, glucotetrasaccharides (HEX4)).

Pharmaceutical compositions can be prepared as injectable formulations or as topical formulations to be delivered to the subject by transdermal transport. Numerous formulations for both intramuscular injection and transdermal transport have been previously developed and can be used in the practice of the invention. The recombinant viral vector can be used with any pharmaceutically acceptable carrier and/or excipient for ease of administration and handling.

### II. Methods of Use

In various aspects, the present disclosure is directed to two complementary methods of targeted synaptic pruning and editing. A first set of methods disclosed herein enables selective ablation and removal of a postsynaptic terminal on a single neuron (irrespective of a presynaptic partner). These methods enable, for example, pruning (or reducing) the overall number of excitatory or inhibitory inputs onto a single neuron. A second set of methods disclosed herein enables targeted ablation and removal of an entire synapse, where the synapse comprises a presynaptic terminal, a postsynaptic terminal and the intervening space (synaptic cleft). These methods enable, for example, targeted disruption and removal of specific neuronal circuits. Further methods are provided for visualizing synapses. These methods may then be applied in various therapeutic methods to correct or treat synaptic disorders including but not limited to depression or other psychiatric disorders.

### (a) Methods of Selectively Ablating a Postsynaptic Terminal

Provided herein is a method of selectively removing or ablating a postsynaptic terminal on a neuron. In general, the method comprises expressing a fusion protein provided herein in a target neuron such that the N terminus of the fusion protein is localized to a postsynaptic terminal of the neuron and then contacting the neuron with an activated micoglia such that the microglia selectively removes or ablates the postsynaptic terminal.

In various aspects, the postsynaptic terminal is an excitatory terminal. When the methods comprise targeting an excitatory postsynaptic terminal, the fusion protein expressed in the target neuron can comprise, in addition to the activated glial receptor binding domain described above, a transmembrane domain derived from a protein that localizes to excitatory postsynaptic terminals. For example, in various aspects, the fusion protein can comprise a transmembrane domain derived from a Shisa6 protein. In some aspects, the fusion protein can comprise a full Shisa6 protein. Exemplary fusion proteins that may be expressed in methods of targeting excitatory postsynaptic terminals include those comprising any of SEQ ID NOs: 9-11 and 15-17.

In further aspects, the postsynaptic terminal is an inhibitory terminal. When the methods comprise targeting an inhibitory postsynaptic terminal the fusion protein expressed in the target neuron can, in addition to the activated glial receptor binding domain described above, comprise a transmembrane domain derived from a protein associated with the inhibitory postsynaptic terminal. For example, in various aspects, the fusion protein can comprise a transmembrane domain derived from a Shisa7 protein. In some aspects, the fusion protein can comprise a full Shisa7 protein. Exemplary fusion proteins that may be expressed in methods of targeting inhibitory postsynaptic terminals include those comprising any of SEQ ID NOs: 12-14 and 18-20.

### (b) Methods of Selective Ablating a Circuit Specific Synaptic Connection (Synapse)

Also provided herein are methods of selectively removing or ablating a synaptic connection (i.e., a synapse) between two neurons where the synaptic connection (synapse) comprises a presynaptic terminal, a postsynaptic terminal and a synaptic cleft between the two. In general, methods of removing an entire synapse comprise expressing a pair of fusion proteins, such as the fusion protein system described herein, where one is expressed in the presynaptic terminal and the other is expressed in the postsynaptic terminal.

Accordingly, in In various aspects, the method of selectively removing or ablating a synaptic connection between two neurons, where the synaptic connection comprises a postsynaptic terminal of a first neuron, a presynaptic terminal of a second neuron and a synaptic cleft between the postsynaptic terminal and the presynaptic terminal can comprise, in part:
(a) delivering to the presynaptic neuron a nucleic acid encoding a first fusion protein, where the first fusion protein comprises an N terminal domain comprising a first fragment of a activated glial receptor binding domain and a C terminal domain comprising a transmembrane domain of a presynaptic protein; and
(b) delivering to the postsynaptic neuron a nucleic acid encoding a second fusion protein, wherein the second fusion protein comprises an N terminal domain comprising a second fragment of an activated glial receptor binding domain and a C terminal domain comprising transmembrane domain of a postsynaptic protein; where the first fragment of the activated glial receptor binding domain of (a) can associate and/or complex with the second fragment of the activated glial receptor binding domain of (b) to form a functional activated glial receptor binding domain. As used herein the term “functional” when used in context of an “activated glial receptor binding domain” means that the resulting “activated glial receptor binding domain” can effectively bind to a target receptor expressed on an activated glial cell.

Suitable first fusion proteins and second fusion proteins for use in this method are provided above (see e.g., Section I-(b)). Other variants of the proteins are envisioned.

Once the nucleic acids encoding the first and second fusion protein have been delivered to the appropriate neurons, as described above, the method continues and further comprises, in part:
(c) expressing the first fusion protein in the postsynaptic terminal of the first neuron such at the first fragment of the activated glial receptor binding domain is localized to the synaptic cleft;
(d) expressing the second fusion protein in the second neuron such that the second fragment of the activated glial receptor binding domain is localized to the synaptic cleft.

In various aspects, ensuring that the first and second fragment of the activated glial receptor binding domain are expressed and localized to the synaptic cleft (especially in the presynaptic terminal) requires targeted delivery of the nucleic acid into the neuron such that the expressed protein travels from the body of the neuron (i.e., soma) through the axon to the axon terminal or dendrites (e.g., dendritic spines). One method of ensuring this is by utilizing a neuron-specific promoter in the gene delivery vector (i.e., AAV), which can drive the expression of the protein specifically in neurons and facilitate its transport to the appropriate subcellular compartments by fusing each fragment to presynaptic (i.e., Synaptophysin) or postsynaptic proteins (i.e., Shisa6, Shisa7).

Once the first and second fusion proteins are expressed in the appropriate locations (e.g., in the presynaptic terminal and the postsynaptic terminal, respectively), the method continues and further comprises:
(e) allowing the first fragment of the activated glial receptor binding domain to complex or associate with the second fragment of the activated glial receptor binding domain to form a functional activated glial receptor binding domain; and
(f) contacting the synaptic connection with an activated microglial cell, wherein the activated microglial cell binds to the functional activated glial receptor binding domain, thereby selectively ablating the synaptic connection between the two neurons.

### (c) Activating Microglia

In various aspects, the methods described herein comprise contacting a synaptic connection (synapse) or postsynaptic terminal with an activated microglial cell. Accordingly, in various aspects, the methods may further comprise activating the microglial cell. In some aspects, the microglial cell may be proximal to the target synaptic connection (synapse) and/or postsynaptic terminal, that is, it may be a proximal glial cell. As used herein, the term “proximal glial cell” refers to glial cells expressing receptors that are physically capable of binding to the activated glial receptor binding domain that forms part of the fusion proteins herein. In other aspects, the microglial cell may be neighboring to a proximal glial cell—that is, it may be a “neighboring glial cell”. As used herein, the term “neighboring glial cell” is understood to mean glial cells that are not located close enough to the target synapse to physically bind or associate with the activated glial receptor binding domain portion of the fusion protein, but that nevertheless communicate or signal to proximal glial cells such that stimulating the neighboring glial cell indirectly leads to an interaction between a receptor on a proximal glial cell and the fusion protein expressed in the target synapse.

Accordingly, in various aspects the methods herein may further comprise activating a proximal glial cell. In other aspects, the methods herein may further comprise activating a neighboring glial cell. In other aspects, the methods herein may further comprise activating a neighboring glial cell and a proximal glial cell.

Methods of activating a glial cell are known in the art. In some aspects, delivery of the nucleic acids in the methods herein may also serve as the trigger for activating the proximal or neighboring glial cells. This is because AAV vectors, which are considered relatively safe and widely used in gene therapy applications, have been shown to exert immune responses in the brain including inflammation and glial cell activation in a dose-dependent manner (e.g., see Lowenstein, P. R., et al., Current gene therapy 7, 347-360, doi:10.2174/156652307782151498 (2007), which is incorporated herein by reference in its entirety). Importantly, the AAV vector can be designed to induce a minor glial response, sufficient to activate glial cells without causing excessive inflammation. In various aspects, when activated by AAV vector administration, microglia express IBA+ as a marker of activation. Accordingly, IBA may be used as a cellular marker to measure successful activation in accord with various aspects of the present disclosure. Additionally, the activation of glial cells may lead to the release of trophic factors that support neuronal survival and synaptic plasticity. By carefully controlling the extent and duration of glial cell activation using AAV vectors (i.e., Gas6 expressing AAV vectors), it may be possible to harness these beneficial effects while minimizing potential adverse outcomes, such as excessive inflammation or bystander damage to healthy synapses.

### (d) Methods of Visualizing Synapses

In addition to the foregoing, the present disclosure also provides for methods of visualizing a target synapse between two neurons. In various aspects, this method comprises: (a) delivering to the presynaptic neuron a nucleic acid encoding a first fusion protein, where the first fusion protein comprises an N terminal domain comprising a first fragment of a fluorophore and, optionally, a first fragment of a activated glial receptor binding domain and a C terminal domain comprising a transmembrane domain of a presynaptic protein; and (b) delivering to the postsynaptic neuron a nucleic acid encoding a second fusion protein, wherein the second fusion protein comprises an N terminal domain comprising a second fragment of the fluorophore and, optionally, a second fragment of an activated glial receptor binding domain and a C terminal domain comprising transmembrane domain of a postsynaptic protein; where: (i) the first fragment of the fluorophore of (a) can associate and/or complex with the second fragment of the fluorophore of (b) to form a functional fluorophore; and (ii), if included, the optional first fragment of the activated glial receptor binding domain of (a) can associate and/or complex with the optional second fragment of the activated glial receptor binding domain of (b) to form a functional activated glial receptor binding domain. As used herein the term “functional” when used in context of an “fluorophore” means that the resulting “fluorophore” can effectively release a detectable fluorescent signal. As used herein the term “functional” when used in context of an “activated glial receptor binding domain” means that the resulting “activated glial receptor binding domain” can effectively bind to a target receptor expressed on an activated glial cell.

In various aspects, the method further comprises (c) expressing the (c) expressing the first fusion protein in the postsynaptic terminal of the first neuron such at the first fragment of the fluorophore is localized to the synaptic cleft; (d) expressing the second fusion protein in the second neuron such that the second fragment of the fluorophore is localized to the synaptic cleft; (e) allowing the first and second fragment of the fluorophore to complex to form a functional fluorophore and (f) detecting an optical signal released by the fluorophore, thereby visualizing the synapse.

Suitable pairs of fusion proteins that may be used in the methods of visualizing synapses herein are provided herein above (e.g., see Section I(b)). Other fusion proteins are contemplated.

### (e) Methods of Treating Synaptic Disorders

Depression is the leading cause of disability worldwide with significant social and economic impact. The limitations of depression treatment are mainly due to the lack of information on the pathophysiology and absence of circuit-specific antidepressants. Many common antidepressants (like SSRIs and ketamine) affect a broad range of brain regions that differentially or oppositely regulate depression phenotypes.

In the brain reward circuitry, NAc, a key brain reward region in the ventral striatum, is most often associated with the rewarding and motivational effects, which are reduced in depression. More than 95 percent of neurons in the NAc are primarily composed of two medium spiny neuronal (MSN) subtypes enriched with dopamine D1 or D2 receptors, respectively, which have opposing roles in reward, action-value, reinforcement, and responses to drugs of abuse.

In chronic stress conditions, excitatory transmission, and intrinsic excitability in the NAc are modulated in MSN subtype-specific manner. It has been known that the ventral hippocampus (vHIP) sends projections to the NAc and the circuit directly modulates social interaction behaviors, which are reduced in depressed mice and more importantly, the vHIP-NAc glutamatergic circuit delivers aversive information only to D1-MSN, but not D2, and increased synaptic strength of the excitatory synapses directly increased depression-like behaviors.

Accordingly, in some aspects, a method herein is provided for treating and/or correcting a synaptic disorder in a subject, the method comprising selectively removing or ablating a postsynaptic terminal in at least one neuron of a subject, according to the methods provided herein (e.g., see Section (II)(a)). In other aspects, a method is provided herein for treating and/or correcting a synaptic disorder in a subject, the method comprising correcting or ablating a synaptic connection (synapse) in a brain of the subject according to the methods provided herein (e.g., see Section (II)(b)).

In various aspects, the synaptic disorder may comprise depression, anydonia, or any other psychiatric disorder. In various aspects, the subject may be a human or may be a mouse model of a human psychiatric disorder. For example, the subject may comprise a mouse subject to a social stress (i.e., to model depression), in accord with methods in the art.

### (III) Definitions

In various aspects, the synaptic disorder may comprise depression, anydonia, or any other psychiatric disorder. In various aspects, the subject may be ahuman or may be a mouse model of a psychiatric disorder. For example, the subject may comprise a mouse subject to a social stress, in accord with methods in the art.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, the terms “prevent,” “preventing,” “prevention,” “prophylactic treatment” and the like refer to reducing the probability of developing a disease, disorder or condition (e.g., stroke) in a subject, who does not have, but is at risk of or susceptible to developing a disease, disorder or condition. The term “effective amount” or “therapeutically effective amount” refers to an amount sufficient to effect beneficial or desirable biological and/or clinical results. In other words, in an aspect, preventing stroke, brain damage (including damage to neurons, axons, or glial) is intended. The words “prevent” and “preventing” and “prevention” also refer to prophylactic or preventative measures for protecting or precluding a subject (e.g., an individual) not having brain damage from progressing to that complication (e.g., after suffering a stroke).

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample can be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It can be caused by an external factor, such as an infectious disease, or by internal dysfunctions, such as stroke, infarction, and the like.

“Contacting” as used herein, e.g., as in “contacting a sample” refers to contacting a sample directly or indirectly in vitro, ex vivo, or in vivo (i.e., within a subject as defined herein). Contacting a sample can include addition of a compound (e.g., a nucleic acid and/or vector as provided herein) to a sample, or administration to a subject. Contacting encompasses administration to a solution, cell, tissue, mammal, subject, patient, or human. Further, contacting a cell includes adding an agent to a cell culture.

As used herein, the term “therapeutic agent” means an agent utilized to treat, combat, ameliorate, prevent or improve an unwanted condition or disease of a subject, such as stroke and associated complications.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient).

As used herein, the term “sequence identity” refers to the number of identical or similar residues (i.e., nucleotide bases or amino acid) on a comparison between a test and reference nucleotide or amino acid sequence. Sequence identity can be determined by sequence alignment of nucleic acid to identify regions of similarity or identity. As described herein, sequence identity is generally determined by alignment to identify identical residues. Matches, mismatches, and gaps can be identified between compared sequences. Alternatively, sequence identity can be determined without taking into account gaps as the number of identical positions/length of the total aligned sequence×100. In one non-limiting embodiment, the term “at least 90% sequence identity to” refers to percent identities from 90 to 100%, relative to the reference nucleotide or amino acid sequence. Identity at a level of 90% or more is indicative of the fact that, assuming for exemplary purposes a test and reference oligonucleotide or length of 100 nucleotides are compared, no more than 10% (i.e., 10 out of 100) of the nucleotides in the test oligonucleotide differ from those of the reference oligonucleotide. Differences are defined as nucleic acid or amino acid substitutions, insertions, or deletions.

As used herein, “operably linked” means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, “activated glial receptor binding domain” refers to a domain of a protein that is capable of binding to and activating a receptor expressed on an activated glia cell. Likewise, an “activated glial receptor binding domain” is capable of being targeted by an activated glia cell (e.g., by binding to one or more receptors on the surface of the activated glia cell). Exemplary “activated glial receptor binding domains” that are contemplated in the present disclosure include Cd3g peptide, which binds to a CR2 and/or CR3 receptor on activated microglia and the Laminin-G like domains (e.g., LG1/LG2) in Gas6 proteins and peptides which bind to TAM receptors.

As used herein, the term “TAM receptor” refers to a member of a family of receptor tyrosine kinases that has Gas6 as a ligand. Exemplary “TAM receptors” include TYRO3, AXL and MER receptors.

As used herein, the term “transmembrane domain” refers to a domain or portion of a transmembrane protein that traverses a plasma membrane. In various aspects, a transmembrane domain can also comprise portions or all of the C terminus and/or N terminus of the portion of the domain embedded in the membrane (i.e., that are located inside or outside the cell).

As used herein, the term “fusion protein” refers to a protein or peptide that comprises at least two domains originally obtained from separate proteins.

As used herein, the term “synapse” refers to a connection between two neurons where neurotransmission occurs and comprises a presynaptic terminal (located on one neuron), a postsynaptic terminal (located on the second neuron) and a synaoptic cleft (space between the two). Synapse is used herein interchangeably with “synaptic connection”. Likewise, the term “synaptic protein” refers to any protein expressed and localized to a synapse.

As used herein, the term “presynaptic terminal” refers to a portion of a neuron, typically at the end of an axon, where vesicles of neurotransmitter are released into a synaptic cleft. Likewise, the erm “presynaptic protein” refers to any protein expressed and localized to a presynaptic terminal.

As used herein, the term “postsynaptic terminal” refers to a portion of a neuron, typically on a dendrite, where neurotransmitter receptors are expressed and where chemical transmission released into a synapse by a first neuron (presynaptic neuron) is detected and propagated into a second neuron (postsynaptic neuron). Likewise, the term “postsynaptic protein” refers to any protein expressed or localized to a postsynaptic terminal.

Unless otherwise defined, all technical terms used herein have the same meaning as commonly understood by one of ordinary skill in the art to which this disclosure belongs.

### EXAMPLES

### Introduction to Examples

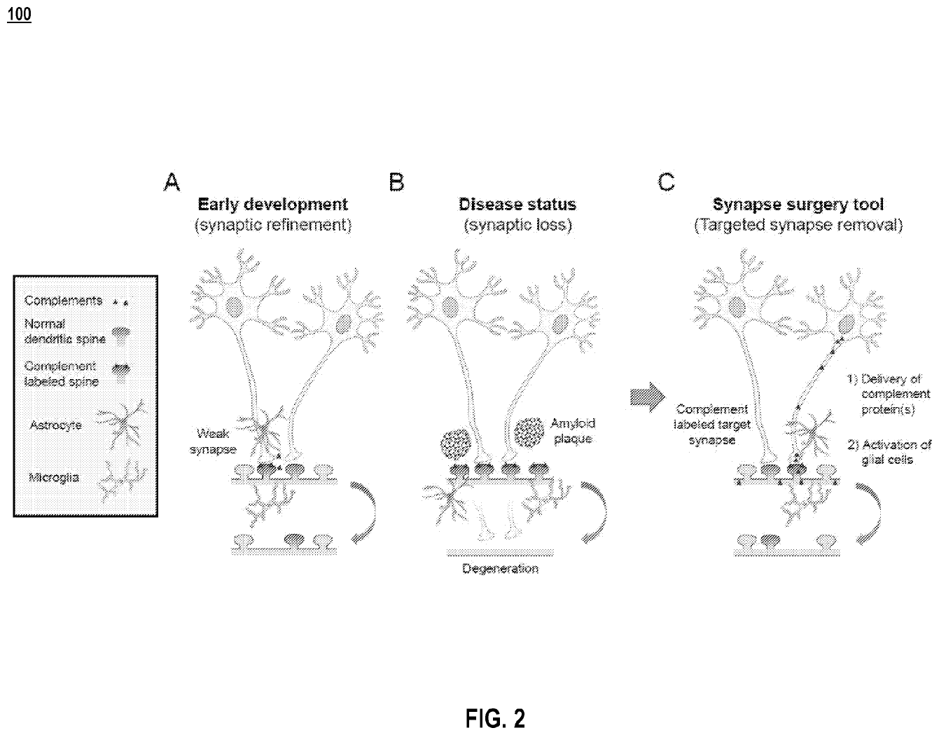

Growing evidence shows that neuron-glia interactions are crucial to refining synaptic connections. Recently, it has been reported that glial cells are intimately involved in neuronal connectivity at the level of synapse formation and pruning. Stevens and colleagues revealed that the classical complement system in the brain is crucial to synaptic pruning mechanisms (FIG. 2A). Complement proteins like C1q and C3 mark synapses for elimination. The complement system is not only involved in developmental processes but also observed in neurodegenerative diseases like Alzheimer's disease which leads to uncontrolled loss of synapses (FIG. 2B). In the following examples, new synapse surgery tools are described to induce synaptic pruning in a targeted and focused matter. As described in FIG. 2C, and further in the following examples, these synapse surgery tools exploit components of complement or other systems that interact with microglia to the synapse to allow for native ablation and removal of targeted synapses. Specifically, by tagging desired synapses with specific complement (or similar) protein(s) and locally activating glial cells, the target synapses can be selectively removed by the endogenous synapse pruning mechanism. As described further below, these methods rely on two conditions. First, the complement proteins (or other proteins that interact with the microglia) and fusion proteins containing them must be localized to the membrane of excitatory or inhibitory synapses (that is, circuit specific synapse labeling with complement proteins), Second, glial cells must be activated in close proximity to the targeted synapses to induce the targeted deletion and removal of the synapse (that is, activation of glial cells in the adult brain).

The examples below describe methods where fusion proteins applying both the complement system and the Gas6-TAM signaling pathway to target and ablate specific synapses in vivo and in vitro. Both the complement system and Gas6-TAM signaling play key roles in regulating immune responses in the CNS. While the complement system can have both pro-inflammatory and protective effects, depending on the context, Gas6-TAM signaling is generally associated with anti-inflammatory and pro-resolving functions. Dysregulation of either pathway could contribute to the development and progression of various neurological disorders.

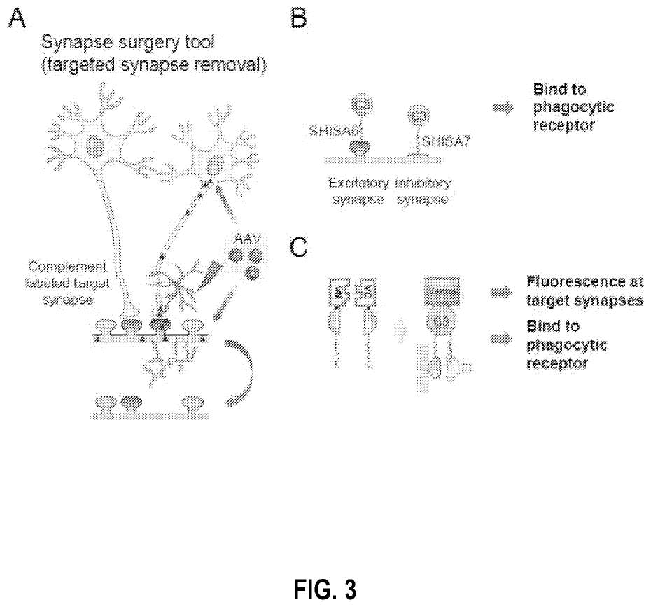

The following examples describe experiments covering three aspects of this targeted synaptic surgery. First, as shown in FIG. 3A, AAV vectors can serve a dual purpose for both complement protein (C3) expression and glial cell activation. As shown in FIG. 3B, selection of specific postsynaptic proteins (e.g., Shisa6 or Shisa7) allow for specific targeting of excitatory or inhibitory synapses. Finally, as shown in FIG. 3C, a protein fragment complementation strategy is also proposed to both visualize and ablate entire synapses (rather than just postsynaptic terminals).

In general, the successful development of the synapse surgery tools described in these examples enables remodelling of specific neural circuits at the synaptic level and determining underlying mechanisms of neuronal function and related diseases. The disclosed tools will enable mechanistically defining specific neuronal circuits that cause neurological diseases and use them as therapeutic options. Further, permanently removal of malfunctioning neural connections can be performed using the disclosed. The disclosed synapse surgery tools are highly specific for target synapses. Combined with existing optogenetic and electrophysiological approaches, synapse surgery tools will delineate precise neural circuit functions in the brain. Further the disclosed tool can be applied to may brain disorders and mental illnesses (so-called synaptopathies including depression, and will provide gene therapeutic strategies for neurological and psychiatric disorders.

Additionally the disclosed tools can be used to study fundamental mechanisms of neuronal functions and related diseases. When a specific connection of neuronal circuits that are causes of neurological diseases is determined, synapse surgery would be a good solution to permanently remove faulty synapses to restore neuronal circuits and relieve the symptoms. In conjunction with gene therapy methods, the disclosed tools can be optimized to be adminstered therapeutically in subjects with autism, depression, and other neural circuit-related disorders of the brain.

### Example 1—Design and Testing of Targeted Synaptic Tools Using Complement Proteins

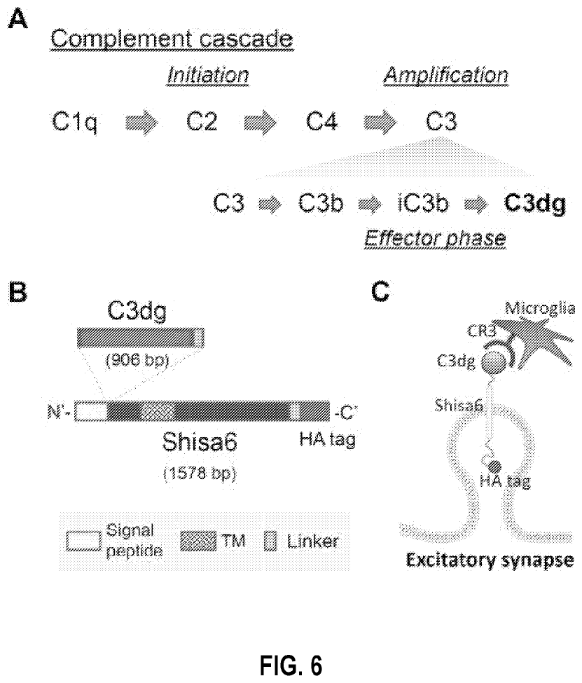

The complement cascade consists of multiple components and their processed forms, and complement C1q is a multi-protein complex. Among them, complement C3 is an end-stage effector in the complement cascade (FIG. 6A). Among the processed forms of C3, C3dg has a simple structure and small size, which constitutes the minimal binding domain of C3. C3dg has specified binding activity to CR3, which mediates phagocytosis i.e. synapse engulfment in microglia. Further, since C3dg is an end-stage opsonin (FIG. 6A), that still retains binding affinity to CR3, overexpression of it is expected to minimize unwanted, divergent effects from the complement cascade activation by upstream complements. To target the C3dg to synapses, various synaptic molecules was considered. Overexpressed Shisa6 localized in the glutamatergic synapses of cultured neurons and D1 neurons in the NAc1, so it was selected as a possible excitatory synapse specific target protein.

A fusion protein was designed where the complement C3dg was fused with Shisa6 to target excitatory synapses which are abundant in various brain regions. The resulting fusion protein was encoded by a nucleic acid where a coding sequence of C3dg was inserted into the N-terminal of Shisa6, immediately after its signal peptide region (FIG. 6B). The C3dg-Shisa6 fused protein was tagged with HA epitope for validation and subcloned into Cre-dependent cassette with double inverted repeat (DIO) of AAV vector, driven by a neuron specific promoter (AAV-Syn1-DIO) ((AAV-DIO-C3dg-Shisa6, SEQ ID NO: 33, FIG. 15, and Table 3).

TABLE 3
AAV-DIO-C3dg-Shisa6 Vector

| | | Size | | | |
|---|---|---|---|---|---|
| Name | Position | (bp) | Type | Description | Notes |
| 5′ ITR | 1-141 | 141 | ITR | AAV 5′ inverted | Allows rescue of virus |
| | | | | terminal repeat | from recombinant |
| | | | | (functional | plasmid and |
| | | | | equivalent of | replication of the viral |
| | | | | wild-type 5′ | genome; this ITR is |
| | | | | ITR) | identical to that of the |
| | | | | | wild-type AAV2 |
| | | | | | genome. |
| SYN1 | 169-637 | 469 | Promoter | Human | Tissue specificity: |
| | | | | synapsin I | Brain. Cell type |
| | | | | promoter | specificity: Mature |
| | | | | | neurons. |
| Lox2272 | 662-695 | 34 | Miscellaneous | Mutated Lox | Recognition site of |
| | | | | site with two | Cre recombinase; |
| | | | | base | can't work with other |
| | | | | substitutions of | types of Lox site. |
| | | | | LoxP | |
| LoxP | 728-761 | 34 | Miscellaneous | Locus of | Recognition site of |
| | | | | X(cross)-over | Cre recombinase. |
| | | | | in P1 | |
| {C3dg- | complement | 2670 | ORF | Signal peptide | SEQ ID NO: 29 |
| Shisa6}/ | (774-3443) | | | (mShisa6 1- | |
| HA | | | | 30aa) - C3dg - | |
| | | | | mShisa6 (31- | |
| | | | | 525) | |
| Kozak | complement | 6 | Miscellaneous | Kozak | Facilitates translation |
| | (3444-3449) | | | translation | initiation of ATG start |
| | | | | initiation | codon downstream of |
| | | | | sequence | the Kozak sequence. |
| Lox2272 | complement | 34 | Miscellaneous | Mutated Lox | Recognition site of |
| | (3456-3489) | | | site with two | Cre recombinase; |
| | | | | base | can't work with other |
| | | | | substitutions of | types of Lox site. |
| | | | | LoxP | |
| LoxP | complement | 34 | Miscellaneous | Locus of | Recognition site of |
| | (3522-3555) | | | X(cross)-over | Cre recombinase. |
| | | | | in P1 | |
| WPRE | 3586-4183 | 598 | Miscellaneous | Woodchuck | Enhances virus |
| | | | | hepatitis virus | stability in packaging |
| | | | | posttranscriptional | cells, leading to |
| | | | | regulatory | higher titer of |
| | | | | element | packaged virus; |
| | | | | | enhances higher |
| | | | | | expression of |
| | | | | | transgenes. |
| BGH pA | 4214-4421 | 208 | PolyA_signal | Bovine growth | Allows transcription |
| | | | | hormone | termination and |
| | | | | polyadenylation | polyadenylation of |
| | | | | signal | mRNA transcribed by |
| | | | | | Pol II RNA |
| | | | | | polymerase. |
| 3′ ITR | complement | 141 | ITR | AAV 3′ inverted | Allows rescue of virus |
| | (4429-4569) | | | terminal repeat | from recombinant |
| | | | | | plasmid and |
| | | | | | replication of the viral |
| | | | | | genome; this ITR is |
| | | | | | identical to that of the |
| | | | | | wild-type AAV2 |
| | | | | | genome. |
| Ampicillin | 5486-6346 | 861 | ORF | Ampicillin | Allows E. coli to be |
| | | | | resistance | resistant to ampicillin. |
| | | | | gene | |
| pUC ori | 6517-7105 | 589 | Rep_origin | pUC origin of | Facilitates plasmid |
| | | | | replication | replication in E. coli; |
| | | | | | regulates high-copy |
| | | | | | plasmid number |
| | | | | | (500-700). |

As shown in FIG. 6C, when delivered and expressed in a neuron, C3dg-Shisa6 is expected to localize to the plasma membrane (via the Shisa6 transmembrane domain) and the C3dg domain is positioned outside the neuron so that it can interact with (bind) an activated microglia expressing its receptor (CR3 receptor).

### Example 2: Microglia are Activated by AAV In Vitro and In Vivo

The AAV vector system is widely used for transgene expression in neuroscience studies and is considered a useful gene therapy vector due to its wide tropism, non-pathogenicity, and low immunogenicity. Despite its relatively marginal immune responses, the AAV vector also exerts immune responses in the brain including inflammation and glial cell activation in a dose-dependent manner. In low-dose intracranial injections with about 10⁸ particles, there are no or little detectable immune responses. On the other hand, higher titer of about 10¹⁰ particles induced significant, but transient immune responses including GFAP responses. These doses are within the range of generally used titers in intracranial viral injection using AAVs. It was speculated that the AAV injection, which was and will be used for complement protein delivery, can be also used for transient activation of glial cells to eliminate labeled synapses at target areas. The acute inflammatory responses may differ between serotypes and virus titers. The glial cell activation patterns after the AAV injection will be examined by immunohistochemistry and flow cytometry to elucidate optimum conditions for synapse elimination by adjusting titer, serotype, and dose. Screening of serotypes will be restricted to serotypes that show neuronal tropism (for e.g., AAV1, 2, 4, 5, 7, 8, and 9). The ratio of activated glial cells will be measured by flow cytometry methods. Transgene construct expression and glial activation will be assessed using tissue clearing and 3-dimensional imaging to measure circuit-wide structural and functional measures.

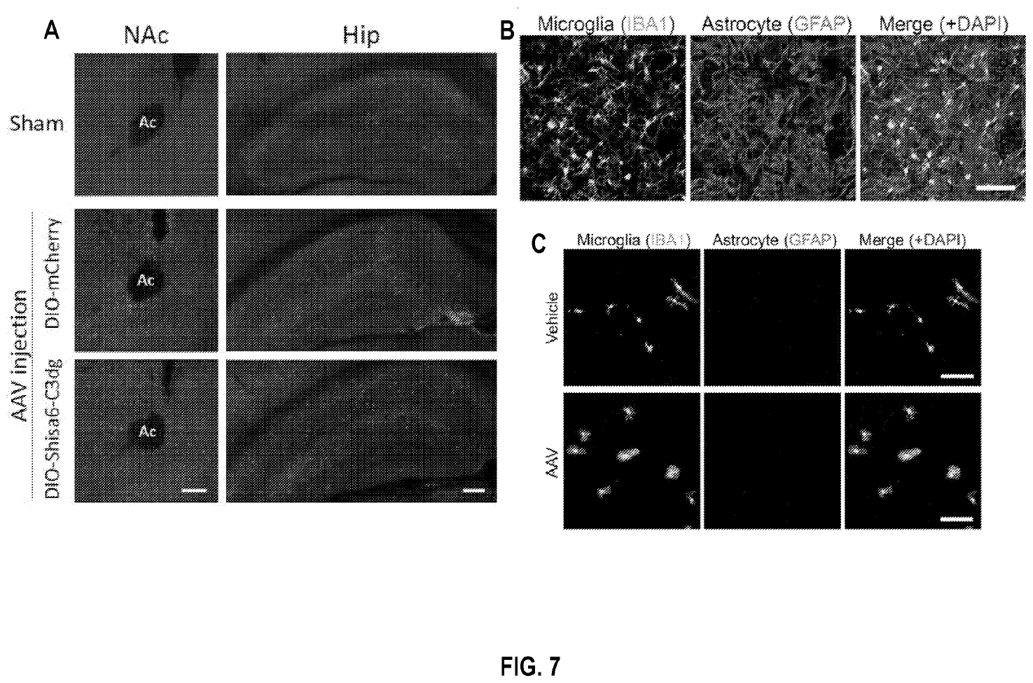

As an initial test to determine whether microglia are activated after AAV delivery, microglia activation in the presence or absence of AAV vectors was tracked in vitro and in vivo. In a first set of experiments, was found that was found that AAV induces microglial activation in vivo in treated animals. Specifically, the activation of microglia was validated using a marker gene, IBA1. The AAV virus (designed in Example 1, above) was introduced in two brain regions, NAc and hippocampus (Hip) to see if the microglial activation occurs upon the AAV injection. It was found that, injection of AAV significantly increased IBA+ microglial cells in both brain regions (FIG. 7A).

In a second set of experiments, microglia were grown on an astrocytic layer using standard methods and labeled with IBA1 (microglia marker), GFAP (astrocyte marker), and DAPI (nucleus marker). Initially isolated (quiescent) microglia form mostly ramified forms. FIG. 7B shows representative images of this mixed primary gilal culture from newborn pups (P1 neonates; DIV 7). Then microglia were isolated from mixed culture by flask tapping and labeled with IBA1. The ameboid glia measured after this stresser were markedly increased 24 hr after AAV treatment (control virus, 2×109 GC/ml) (FIG. 7C).

### Example 3: Synaptic and Circuit Analysis of Cd3g-Shisa6 Construct In Vivo

In the brain reward circuitry, the nucleus accumbens (NAc) is an integration center of various neural inputs related to mood regulation and addiction, which is a good model for validation of circuit specificity. In this example, specificity and validation of the C3dg-Shisa6 construct were tested in a specific neuron type, D1-type neurons, using D1-Cre transgenic mice. In these experiments, effects of the C3dg-Shisa6 expression in synaptic structures and overall connectivity in the nucleus accumbens (NAc) and hippocampus (Hip) were analyzed.

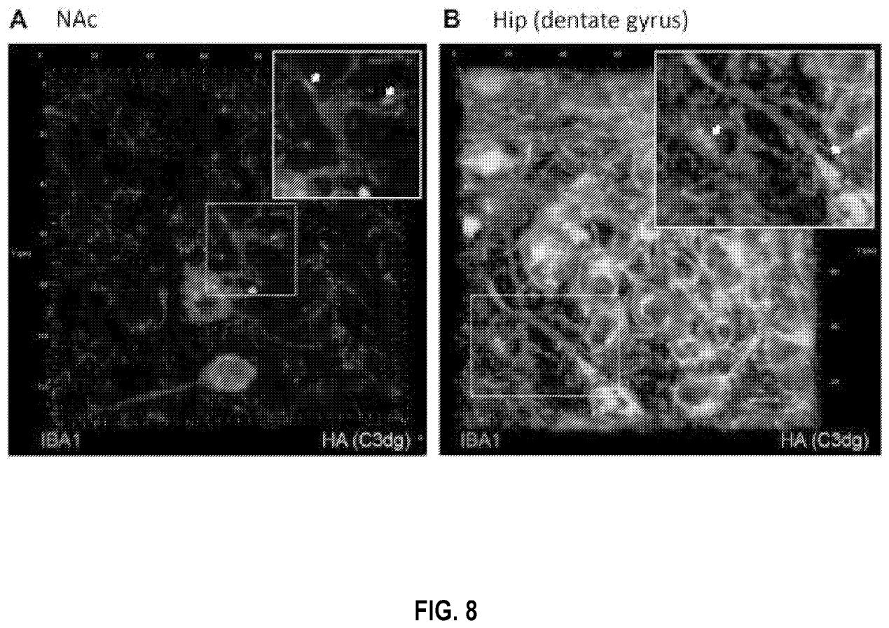

Assessment of spine structures. First, assessment of spine structures were conducted. The synapse engulfment was examined in both brain regions after the expression of C3dg-Shisa6 overexpression. It was found that microglia contained HA labeled speckles within the cells (FIG. 8A-8B).

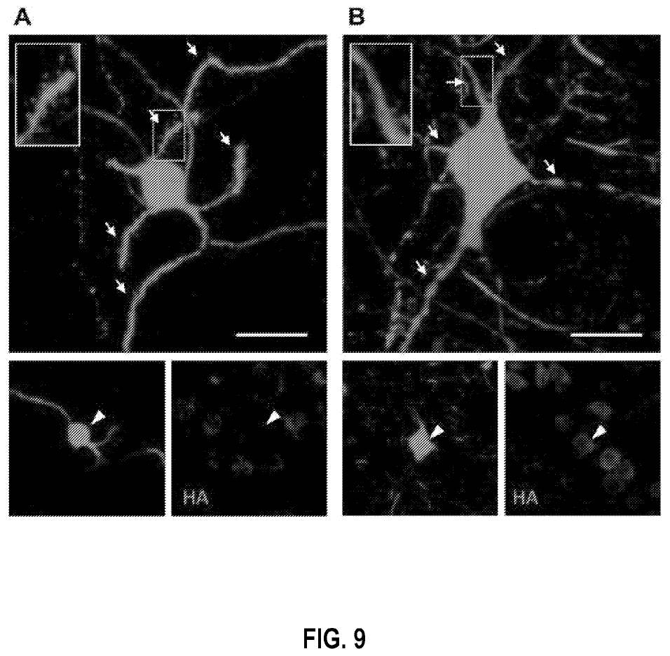

Assessment of neural circuit connectivity. Next, in mice injected with C3dg, C3dg-expressing neurons showed markedly reduced spine density, compared to C3dg-negative neurons (FIGS. 9A and 9B). This suggested that activated microglia act on complement expressing neurons specifically, but not in adjacent neurons without expression of C3dg.

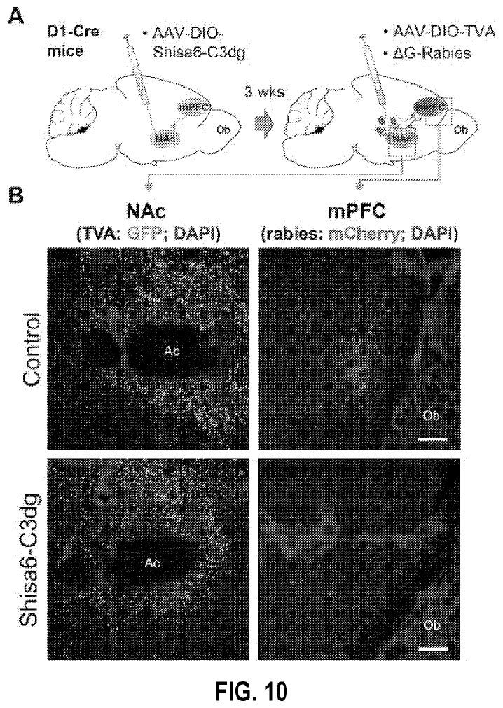

Testing circuit-related behaviors. Finally, further assessment of neural circuit connectivity was performed to examine if the disclosed tool functionally disconnects target circuits. Mono-synaptic retrograde tracing using delta-G rabies virus was employed to validate if the elimination of synapses can block the retrograde labeling of target circuitry. The medial prefrontal cortex (mPFC) is one of the main excitatory inputs to the NAc. Notably, in the C3dg overexpressed mouse, rabies virus-infected projecting neurons were markedly reduced in the mPFC region (FIG. 10A-10B).

### Example 4: Behavioral Assessments of C3dg Expression in D1-Neurons

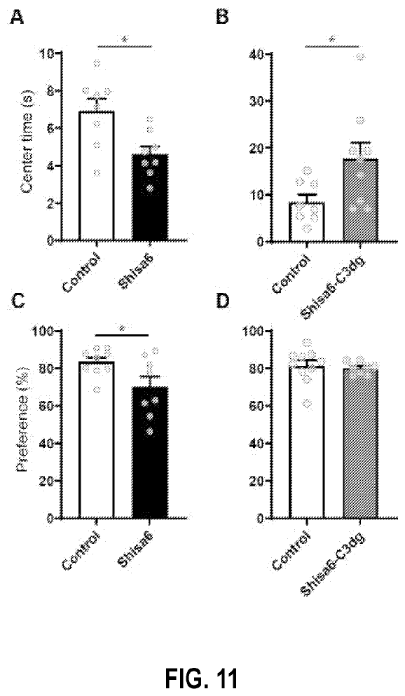

Further testing of circuit-related behaviors were conducted in mice treated with the Shisa6-C3dg vector described above. These circuit-level changes showed that C3dg overexpression modulated anxiety and anhedonia-like behaviors. Shisa6 overexpression in D1 neurons increased anxiety as exhibited by decreased center zone time in open field test, and anhedonia exhibited by decreased sucrose preference, possibly caused by enhanced excitatory synapses via Shisa6 actions on AMPA receptor functions. However, the removal of the target synapses by C3dg-tagged Shisa6 reversed the behavioral changes (FIG. 11A-11D). In summary, these data demonstrated that AAV-mediated expression of complement tag modified the target synapses and that C3dg can be used as an effective tag for synapse surgery tools. Moreover, the binding can be modulated pharmacologically with putative antagonists, simvastatin (a cholesterol-lowering drug), NIF and mAb 10719 during the tool development and experimental applications. More precise studies with quantitative analyses will be performed, in addition to designing circuit-specific tools using fragment complementation as described in the following examples

### Example 5: In Vitro and Ex Vivo Validation of Synapse Surgery Tools: Static or Live Imaging of Synapse Removal in Target Neurons

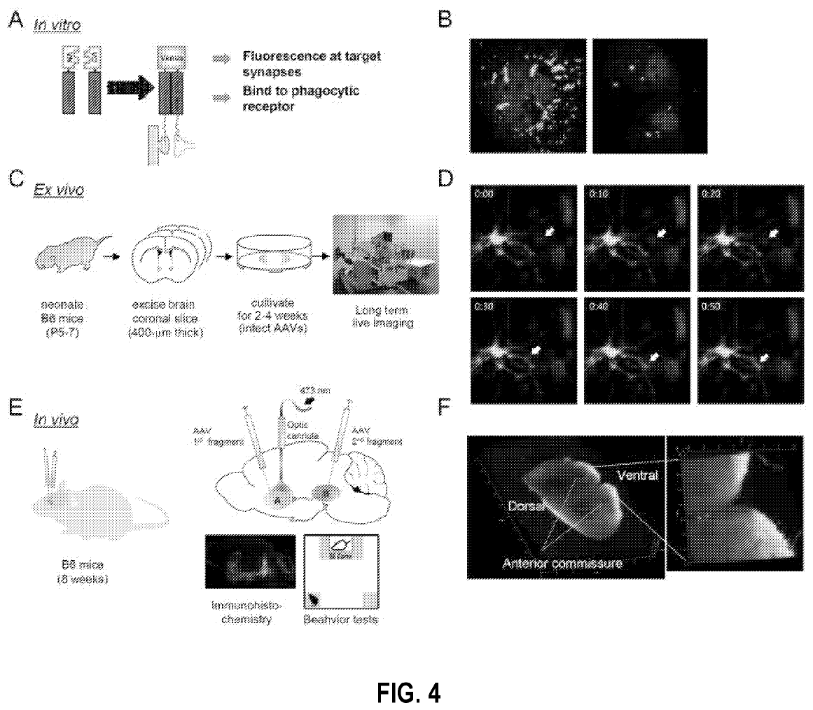

Further to the experiments described in Examples 1-4, in vitro cell line and primary neurons will be used to further demonstrate the activity of designed constructs (FIG. 4A-4B). Further, each complement fragment will be infected in Neuro2A cells and assessed for fluorescence complementations on their contacting membrane. Fluorescent complementation signals for synapse labeling were validated in NIH3T3 cell lines after co-expression of control constructs. Expression in primary neuron-microglia cultures will be assessed by evaluating in vitro maturated primary neuronal cells, for e.g., in neuron-microglia co-culture, for Synapse type-specific localization of Shisa-complement fusion protein or using circuit-specific tools. Cre driver lines was used for cell-type specific validations.

Live-cell imaging of phagocytosis of synapse will be examined in ex vivo brain slice model (FIG. 4C-4D). Engulfment of target synapses which are fluorescence-labeled will be examined in live imaging conditions of brain slices. Time-lapse imaging of hippocampal slices, will be examined for movement of a GFP labeled microglia, using for e.g., LSM 710 confocal system with images captured at 10 min interval, for 2-days. Fluorescence labeled complement and microglia will be imaged simultaneously, to observe synapse ablation events in the live imaging experiments.

In in vivo mouse model, two different brain regions (A & B) will be injected with circuit specific complement AAV pairs in each site. After removal of synapses, optogenetic activation will be applied. Validation of circuit functionality will be assessed by behavior tests and immunohistochemistry using tissue clearing (FIG. 4E). Validation of tissue clearing and antibody staining (CLARITY) using mouse brain section were further conducted (FIG. 4F). A brain/hydrogel block (3-mm thick) containing prefrontal cortex and anterior striatum regions were lipid-cleared and stained with MAP2 antibody to visualize neuronal processes using LSM 710 confocal system. Further, circuit tracing can be performed using monosynaptic retrograde tracing. For e.g., labeling using delta-G rabies virus of afferent input neurons via contacting synapses or circuit-specific synapse labeling by eGRASP fluorescent protein complementation at specific synapses for neural circuits between discrete brain regions can be performed (FIG. 5A-5C). Circuit specificity of synapse surgery tools will be assessed in well characterized circuits of various brain regions for e.g., striatum, hippocampus (DG, dentate gyrus), cortex (RSCg, granular retrosplenial cortex) of the mouse brain. The eliminations of a specific circuit can be easily detected by assessing the distinct spatial patterns of synapses with fluorescence labeling (e.g. eGRASP). Other approaches including tissue clearing (CLARITY) and 3-dimensional imaging can also be applied.

### Example 6: Synapse Surgery Tools Utilizing the Gas6 Signaling Pathway for Targeted Elimination

The Gas6-TAM signaling pathway plays a vital role in the regulation of the immune response, particularly in the context of phagocytosis and the resolution of inflammation. Gas6 signaling through TAM receptors on microglia can suppress the release of pro-inflammatory cytokines and promote the expression of anti-inflammatory mediators, such as interleukin-10 (IL-10). Gas6-TAM signaling has been associated with the “M2” phenotype of microglia, which is characterized by a pro-resolving and anti-inflammatory profile. In this context, Gas6-TAM signaling can be considered to have mainly anti-inflammatory or immune-regulatory effects, contributing to the maintenance of CNS homeostasis. This property may counterbalance the pro-inflammatory effects of complement system activation.

Synapse surgery tools utilizing the Gas6 signaling pathway is designed to enhance the specificity and efficiency of synapse surgery tools for targeted elimination, while also benefiting from its anti-inflammatory effects.

An adeno-associated virus (AAV) construct that incorporates Gas6 LG1/LG2 domains, similar to the existing construct using the C3dg fragment from the complement system was designed. Specifically, the LG1/LG2 domains of GAS6 were used instead of C3dg (FIG. 16, SEQ ID NO: 34, annotated in Table 4 below).

TABLE 4
AAV-DIO-Gas6-Shisa6 Vector

| | | Size | | | |
|---|---|---|---|---|---|
| Name | Position | (bp) | Type | Description | Notes |
| 5′ ITR | 1-130 | 130 | ITR | AAV 5′ inverted | Allows rescue of virus |
| | | | | terminal repeat | from recombinant |
| | | | | (functional | plasmid and |
| | | | | equivalent of | replication of the viral |
| | | | | wild-type 5′ | genome; this ITR is |
| | | | | ITR) | identical to that of the |
| | | | | | wild-type AAV2 |
| | | | | | genome. |
| SYN1 | 158-626 | 469 | Promoter | Human | Tissue specificity: |
| | | | | synapsin I | Brain. Cell type |
| | | | | promoter | specificity: Mature |
| | | | | | neurons. |
| Lox2272 | 651-684 | 34 | Miscellaneous | Mutated Lox | Recognition site of |
| | | | | site with two | Cre recombinase; |
| | | | | base | can't work with other |
| | | | | substitutions of | types of Lox site. |
| | | | | LoxP | |
| LoxP | 717-750 | 34 | Miscellaneous | Locus of | Recognition site of |
| | | | | X(cross)-over | Cre recombinase. |
| | | | | in P1 | |
| {Gas6- | complement | 2754 | ORF | Signal peptide | SEQ ID NO: 30 |
| Shisa6- | (763-3516) | | | (mShisa6 1- | |
| HA}/HA | | | | 30aa) - Gas6 - | |
| | | | | mShisa6 (31- | |
| | | | | 525) | |
| Kozak | complement | 6 | Miscellaneous | Kozak | Facilitates translation |
| | (3517-3522) | | | translation | initiation of ATG start |
| | | | | initiation | codon downstream of |
| | | | | sequence | the Kozak sequence. |
| Lox2272 | complement | 34 | Miscellaneous | Mutated Lox | Recognition site of |
| | (3529-3562) | | | site with two | Cre recombinase; |
| | | | | base | can't work with other |
| | | | | substitutions of | types of Lox site. |
| | | | | LoxP | |
| LoxP | complement | 34 | Miscellaneous | Locus of | Recognition site of |
| | (3595-3628) | | | X(cross)-over | Cre recombinase. |
| | | | | in P1 | |
| WPRE | 3659-4256 | 598 | Miscellaneous | Woodchuck | Enhances virus |
| | | | | hepatitis virus | stability in packaging |
| | | | | posttranscriptional | cells, leading to |
| | | | | regulatory | higher titer of |
| | | | | element | packaged virus; |
| | | | | | enhances higher |
| | | | | | expression of |
| | | | | | transgenes. |
| BGH pA | 4287-4494 | 208 | PolyA_signal | Bovine growth | Allows transcription |
| | | | | hormone | termination and |
| | | | | polyadenylation | polyadenylation of |
| | | | | signal | mRNA transcribed by |
| | | | | | Pol II RNA |
| | | | | | polymerase. |
| 3′ ITR | complement | 130 | ITR | AAV 3′ inverted | Allows rescue of virus |
| | (4502-4631) | | | terminal repeat | from recombinant |
| | | | | | plasmid and |
| | | | | | replication of the viral |
| | | | | | genome; this ITR is |
| | | | | | identical to that of the |
| | | | | | wild-type AAV2 |
| | | | | | genome. |
| Ampicillin | 5559-6419 | 861 | ORF | Ampicillin | Allows E. coli to be |
| | | | | resistance | resistant to ampicillin. |
| | | | | gene | |
| pUC ori | 6590-7178 | 589 | Rep_origin | pUC origin of | Facilitates plasmid |
| | | | | replication | replication in E. coli; |
| | | | | | regulates high-copy |
| | | | | | plasmid number (500- |
| | | | | | 700). |

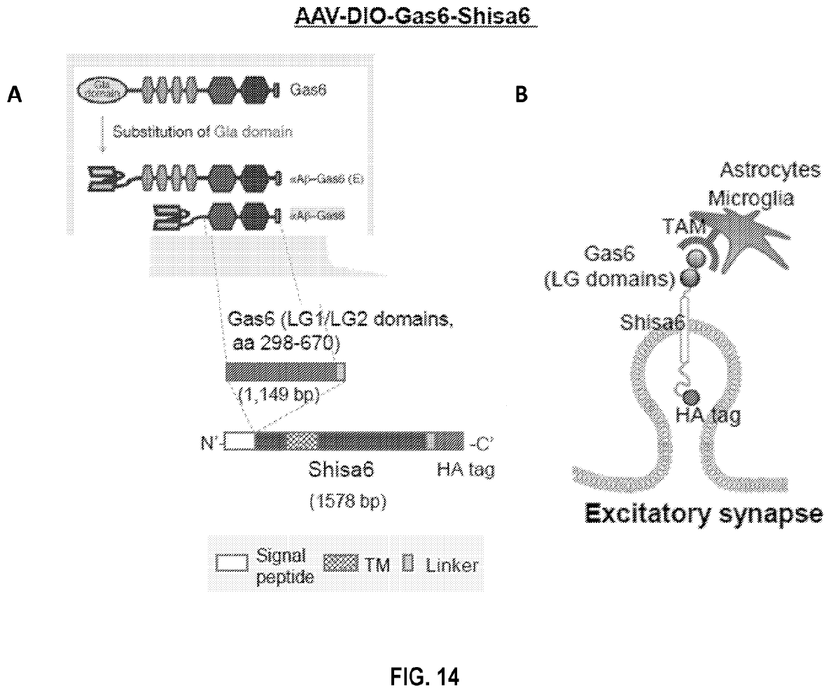

Such AAV constructs will have Gas6 LG domains, aiming to provide both targeted elimination and anti-inflammatory effects (FIG. 14). Gas6-LG can be employed for all designs of this invention described above, replacing the complement C3dg fragment to leverage the benefits of the Gas6 signaling pathway in targeted elimination and inflammation control. Further, the tools utilizing Gas6-LG domains can be used independently or in conjunction with disclosed tools based on complement C3dg to improve the efficiency of synapse surgery while controlling unwanted inflammatory reactions. Combining these tools with those based on the complement C3dg fragment could provide a balanced strategy for modulating synapse elimination and promoting CNS homeostasis.

### Example 7: Circuit-Specific Targeting of Complement Proteins

Based on the structure of C3dg, tools for targeting specific neural circuits using protein fragment complementation strategy were designed. As described above, serial splitted fragments will be tested in silico validation using AlphaFold & in vitro assays. In addition, several antagonists neutrophil inhibitory factor 26 (NIF26), simvastatin, and mAb 10719, which occlude the C3-CR3 interaction can be used to validate the specific interactions (FIG. 12A-12B).

A synapse is a junction between presynaptic and postsynaptic neurons. To locate exogenous complement proteins at the junction of target synapses, a protein-fragment complementation strategy, will be used. Two divided fragments of a complement protein will be expressed in pre- and post-synaptic neurons, respectively. When the two fragments come in contact in proximity at synapses, fragments are brought together and fold into the native structure, a functional complement protein that can be detected by activated glial cells. The fragments will be designed based on complement and complement receptor binding. For example, complement C3dg may be split around the binding motif of the protein (Asp1258), which is recognized by microglial phagocytic receptors CR3 and enhance phagocytosis. Simvastatin, NIF and mAB 107 antagonize the interaction. Further, serial fragmentation of C3dg will be performed to identify the position of binding motif and in silico structural prediction will be considered to choose putative protein fragmentation pairs of C3dg. Split fluorescent proteins will be added to the fragments to label the target synapses with fluorescence for easy visualization (FIG. 12A-12B).

The fragments should form the functionally intact structure of original complement proteins when they are in close proximity to others, and thus would not form a completely folded structure prior to the association between partner peptides. These features can be assessed with in silico protein structural analysis tools, such as Alphafold22, in advance. Every fragment pair will be tested first with in silico analysis and then tested as a synthesized peptide forms in vitro or in vivo conditions.

For presynaptic side, first fragment (e.g. N-terminal fragment) will be sub-cloned into a membrane targeting construct with a signal peptide and transmembrane domain. Cell type-specific expression will be conferred by a neuron-specific promoter (e.g., Synapsin or CamKIIa promoter) or using a Cre-Lox system (e.g., Cre-expressing mouse model and AAV-double inverted repeat (DIO) construct). Such fragments will be injected into the location of the afferent neuronal cell body.

For postsynaptic side, a second fragment (e.g., C-terminal fragment) will be fused to the extracellular domain of SHISA6 or SHISA7 for circuit-specific excitatory or inhibitory synapse elimination. For conferring cell type-specific expression a neuron-specific promoter (e.g., Synapsin or CamKIIa promoter) or Cre-Lox system (e.g., Cre-expressing mouse model and AAV-double inverted repeat (DIO) construct) will be used. Such fragments will be injected into the location of postsynaptic neurons.

### Example 8: In Vivo Validation in a Preclinical Depression Model (Chronic Social Defeat Stress Model)

Using the proposed synapse surgery tools, the depression-related circuitry will be further demonstrated and delineated.

Depression is the leading cause of disability worldwide with significant social and economic impact. The limitations of depression treatment are mainly due to the lack of information on the pathophysiology and the absence of circuit-specific antidepressants. Many common antidepressants like SSRIs and ketamine target a broad range of brain regions that differentially contribute to depression phenotypes. In the brain reward circuitry, NAc, a key brain reward region in the ventral striatum, most often associated with the rewarding and motivational effects, are reduced in depression. More than 95 percent of neurons in the NAc are primarily composed of two medium spiny neuronal (MSN) subtypes enriched with dopamine D1 or D2 receptors, respectively, which have opposing roles in reward, action-value, reinforcement, and responses to drugs of abuse. In chronic stress conditions, excitatory transmission, and intrinsic excitability in the NAc are modulated in MSN subtype-specific manners. The ventral hippocampus (vHIP) sends projections to the NAc and the circuit directly modulates social interaction behaviors, which are reduced in depressed mice and more importantly, the vHIP-NAc glutamatergic circuit delivers aversive information only to D1-MSN, but not D2, and increased synaptic strength of the excitatory synapses directly increased depression-like behaviors.

The functional relevance of D1 type-specific synapses receiving signals from the ventral hippocampus using the synapse surgery tools will be validated.

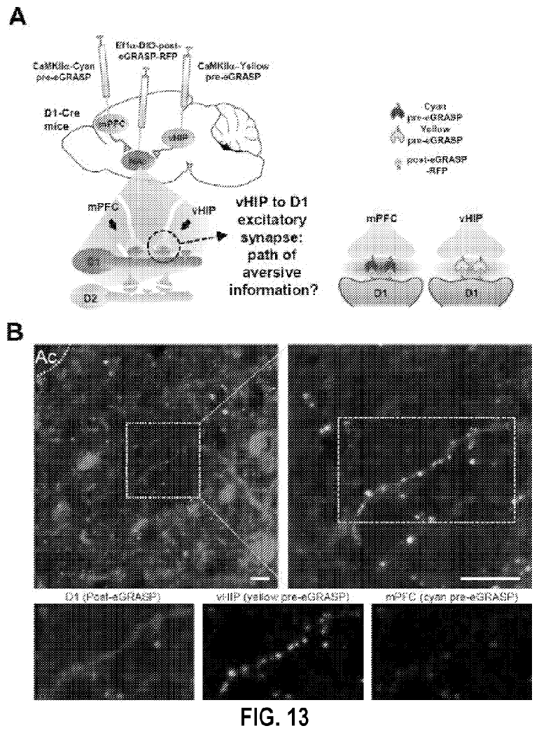

Quantification of afferent input specific synapses of D1-MSNs in the NAc will be performed. The vHIP-D1 synapses in non-stress and stress conditions (chronic social defeat) will be visualized and quantified with circuit-specific synapse labeling technique (eGRASP). The eGRASP construct will be injected in D1-Cre mice (10c′ & 10?) as shown in FIG. 13A. Three weeks after the injection, the mice will be subjected to chronic social defeat stress. Brains of susceptible and resilient mice will be perfused and analyzed for both mPFC and vHIP synapses in the NAc sections. Immunohistochemistry images will be analyzed by ImageJ and Imaris.

Further, it will be examined whether cell-type specific ablation of mPFC or vHIP afferent synapses in D1-MSNs make mice less susceptible to depression- and anxiety-like phenotype. In this experiment, anxiety- and despair-like behaviors in mice where mPFC or vHIP inputs are selectively deleted from D1-MSNs using the synapse surgery tools (10c′ & 10? for each input) will be determined. Additionally, if ablation of mPFC or vHIP afferent synapses in D1-MSNs is sufficient to modulate a baseline anxiolytic or anti-depressant-like phenotype when compared to controls will be determined. Anxiety will be assessed for e.g., using the open field, elevated plus maze, and light-dark box tests. Despair-like behavior will be measured using the forced swim and tail suspension tasks, and anhedonia will be measured with the sucrose preference test. Sucrose preference can be performed by administering 1% sucrose solution and water to the subject mice and calculating the percentage of sucrose preference averaged over three days. Sucrose preference percentage can be calculated as follows:

$$sucrose preference ( \% ) = \frac{Sucrose intake}{total fluid consumed ( sucrose + water )} \times 100$$

Additionally, whether the afferent input specific synapse ablation in D1-MSNs block chronic social defeat-induced depression-like behaviors will be examined. This experiment will be carried out using the same tests as described above, except that the mice will be subjected to the full chronic social defeat stress protocol prior to behavioral testing. Identified susceptible mice will be injected with synapse surgery tools to remove synaptic inputs from mPFC or vHIP. One month after the injection, the subject mice will be tested with the same series of behavior tests.

The electrophysiological properties of afferent input modified D1-MSNs of socially defeated mice will be determined. If selective deletion of afferent synapses in D1-MSNs alters resting membrane potential, firing frequency, or steady-state input resistance will be assessed. Baseline or intrinsic neuronal excitability is a measure of how responsive a neuron is to stimuli. It is hypothesized that SIRT1 will alter intrinsic neuronal excitability in a cell type-specific manner. Mice will be anesthetized with isofluorane and perfused (1.5 to 2 ml/min) 1 min with ice-cold aCSF. Acute brain slices 250 μm thick containing the NAc shell will be made with a vibratome in cold aCSF. Whole-cell voltage-clamp recordings will be obtained from eYFP+ MSNs to measure neuronal intrinsic excitability and rheobase threshold to induce first spike in response to current injections and membrane input resistance in the steady-state current-voltage relationship. All experiments will be carried out blind to the treatment and genotype of mice. For all whole cell experiments, series resistance (Rs) will be monitored throughout recordings; only stable (<15% change) cells with Rs<25 MΩ throughout the recording will be included. Responses will be obtained from 4-6 neurons per animal, with means per animal used to generate group means and to perform statistical analysis. Biocytin will be injected to analyze structure of dendrites and synapses. All morphological and electrophysiological studies will be performed in both males and female as sex may be a potential biological variable.

Circuit specific-synapse labeling in the NAc was performed. The synapses receiving inputs from two major excitatory afferent regions to the NAc, mPFC, and vHIP, were labeled with dual-eGRASP (FIGS. 13A-13B). The synapses will be removed with the synapse surgery tools from the subject mice and perform behavior tests including social interaction, despair- and anxiety-like behaviors will be performed.

### Example 9: Development of Diverse Tools

In addition to these basic ablation strategies, temporal regulation steps can be endowed by adding Tet-on and Tet-off systems to the constructs. Administration of doxycycline can turn on and off the synaptic elimination temporarily, allowing the control the synapse ablation in a specific time window. Combining with behavioral and optogenetics paradigms, precise causal relations between specific synapses and behaviors can be delineated.

Retrograde or anterograde viral approaches also can be considered to target specific circuitry. For example, retrograde AAV (for e.g., rAAV2-retro) which expresses Cre can be combined with Cre-dependent complement expression viruses to specifically label afferent neurons. This approach will target a circuit more specifically and might reduce side-effects caused by overexpression of exogenous complement proteins in other brain circuits.

Further, the synapse surgery tools can be inserted into the mouse genome to create transgenic mouse models. This can be used to create cell-type specific mouse models by crossing various Cre driver lines and minimize viral injections, which might be needed only for glial cell activation. In addition, transgenic animal models can be used for assessing the limitation of cargo sizes for viral packaging.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

1. A fusion protein comprising:
(a) an N terminal domain comprising an activated glial receptor binding domain; and
(b) a C terminal domain comprising a transmembrane domain of a synaptic protein.

2. The fusion protein of claim 1, wherein (a) comprises an activated glial receptor binding domain of a C3 complement protein or an activated glial receptor binding domain of a Gas6 protein.

3. The fusion protein of claim 1, wherein (a) comprises a C3dg peptide.

4. The fusion protein of claim 3, wherein an amino acid sequence of (a) has at least 70%, at least 75%, at least 80%, at least 85%, at least 90%, at least 95%, at least 96%, at least 97%, at least 98%, at least 99% or 100% sequence identity to SEQ ID NO: 1.

5. (canceled)

6. The fusion protein of claim 1, wherein (a) comprises a Laminin-G like domain of Gas6.

7. The fusion protein of claim 6, wherein an amino acid sequence of (a) has at least 70%, at least 75%, at least 80%, at least 85%, at least 90%, at least 95%, at least 96%, at least 97%, at least 98%, at least 99% or 100% sequence identity to SEQ ID NO: 2.

8. (canceled)

9. The fusion protein of claim 1, wherein the synaptic protein is a postsynaptic protein.

10. The fusion protein of claim 9, wherein the postsynaptic protein is Shisa6 or Shisa7.

11. The fusion protein of claim 10, wherein an amino acid sequence of (b) has at least 70%, at least 75%, at least 80%, at least 85%, at least 90%, at least 95%, at least 96%, at least 97%, at least 98%, at least 99% or 100% sequence identity to SEQ ID NO: 3 or 4.

12. (canceled)

13. The fusion protein of claim 1, further comprising a signal peptide or an HA tag.

14. The fusion protein of claim 1, further comprising a peptide linker between (a) and (b).

15. (canceled)

16. The fusion protein of claim 1, comprising an amino acid sequence of any one of SEQ ID NOs: 9 to 20.

17. A nucleic acid encoding the fusion protein of claim 1.

18. An expression vector comprising the nucleic acid of claim 17.

19. The expression vector of claim 18, further comprising a neuron specific and/or Cre-dependent promoter.

20. The expression vector of claim 19, wherein the neuron specific and/or Cre-dependent promoter comprises a Syn1-DIO promoter, a Synapsin promoter, or a CamKIIa promoter.

21. (canceled)

22. The expression vector of claim 18, wherein the vector comprises an AAV vector.

23. A method of selectively removing or ablating a postsynaptic terminal from a neuron, the method comprising:
(a) delivering an expression vector comprising a nucleic acid encoding a fusion protein comprising: (i) an N terminal domain comprising an activated glial receptor binding domain; and (ii) a C terminal domain comprising a transmembrane domain of postsynaptic protein;
(b) expressing the fusion protein so that the activated glial receptor binding domain is localized to a synaptic cleft of the postsynaptic terminal of the neuron; and
(c) contacting the neuron with an activated microglial cell so that the activated microglial binds to the activated glial receptor binding domain and selectively ablates the postsynaptic terminal of the neuron.

24. The method of claim 23, wherein the expression vector of (a) comprises a neuron specific and/or Cre-dependent promoter or an AAV vector.

25. The method of claim 23 or 24, wherein the postsynaptic terminal is an excitatory postsynaptic terminal or an inhibitory postsynaptic terminal.

26. (canceled)

27-47. (canceled)
